(19) United States
(12) Patent Application Publication
(10) Pub. No.: US 2024/0165263 A1
(43) Pub. Date: May 23, 2024

(54) TARGETING MULTIPLE T CELL TYPES USING SPHERICAL NUCLEIC ACID VACCINE ARCHITECTURE

(71) Applicant: NORTHWESTERN UNIVERSITY, Evanston, IL (US)

(72) Inventors: Chad A. Mirkin, Wilmette, IL (US); Michelle Hope Teplensky, Evanston, IL (US); Michael Evangelopoulos, Evanston, IL (US); Shuya Wang, Evanston, IL (US)

(21) Appl. No.: 18/283,699

(22) PCT Filed: Mar. 30, 2022
(86) PCT No.: PCT/US22/22626

§ 371 Date: Sep. 22, 2023

Related U.S. Application Data
(60) Provisional application No. 63/222,869, filed on Jul. 16, 2021.
(60) Provisional application No. 63/167,977, filed on Mar. 30, 2021.

(51) Int. Cl. A61K 47/69; A61K 39/00; A61K 39/385; A61K 47/54; A61K 47/55; A61P 37/04

(52) U.S. Cl. CPC A61K 47/6925; A61K 39/001114; A61K 39/385; A61K 47/549; A61K 47/55; A61K 47/554; A61K 47/6911; A61K 47/6937; A61P 37/04; A61K 2039/55561; A61K 2039/575; A61K 2039/70

(57) ABSTRACT

The disclosure is generally related to spherical nucleic acids (SNAs), nanostructures with a core surrounded by a radial presentation of oligonucleotides, that can target multiple classes of immune cells. Methods of making and using the nanoparticles are also provided herein. In some aspects, the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen.

### CROSS-REFERENCE TO RELATED APPLICATIONS

This application claims the priority benefit under 35 U.S.C. § 119(e) of U.S. Provisional Patent Application No. 63/167,977, filed Mar. 30, 2021, and U.S. Provisional Patent Application No. 63/222,869, filed Jul. 16, 2021, each which is incorporated herein by reference in their entirety.

### STATEMENT OF GOVERNMENT SUPPORT

This invention was made with government support under grant numbers CA208783 and CA199091-03 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-087R_Seqlisting.txt”, which was created on Mar. 30, 2022 and is 9,503 bytes in size. The subject matter of the Sequence Listing is incorporated herein in its entirety by reference.

### FIELD OF THE INVENTION

The disclosure is generally related to spherical nucleic acids (SNAs), nanostructures with a core surrounded by a radial presentation of oligonucleotides, that can target multiple classes of immune cells. Methods of making and using the nanoparticles are also provided herein.

### SUMMARY

Spherical Nucleic Acids (SNAs) are potent immunotherapeutics that are capable of activating the immune system against a specific target. SNAs, which comprise a dense shell of oligonucleotides radially conjugated to a nanoparticle core, have demonstrated that vaccine structure directly influences function and the resulting success of the therapy. This is a powerful concept known as rational vaccinology, and allows for the heightening of immune responses utilizing the same clinically-employed targets, but with architecture leveraged for potency. The immune system is complex and thus vaccines need to activate multiple different types of cells for a robust response and ultimate tumor rejection. The present disclosure describes the synthesis of SNAs with defined placement of multiple different targets that activate multiple different immune cell types, and shows that responses can be enhanced and that structure and placement of the targets within the SNA strongly dictates the vaccine efficacy.

Applications of the technology described herein include, but are not limited to:
Vaccine design
Cancer immunotherapy
Nanomedicine
Treatment of immune-related disorders (e.g., autoimmune disorders).

Advantages of the technology described herein include, but are not limited to:
Structural control of vaccine leading to the controlled presentation of components to cells;
Defined placement of immune targets within nanoparticle and control over intracellular trafficking and kinetics of release after uptake by an immune cell;
Presentation enhances serum stability, cellular uptake, and co-delivery of immune activation and targeting components;
Platform technology with modularity for changing the targets (e.g., cancer targets);
Enhanced immune responses

Accordingly, in some aspects the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen. In some embodiments, the first antigen is encapsulated in the nanoparticle core. In some embodiments, the second antigen is attached to one or more oligonucleotides in the shell of oligonucleotides through a linker. In further embodiments, the second antigen is attached through the linker to an oligonucleotide in the shell of oligonucleotides that is attached to the nanoparticle core. In still further embodiments, the second antigen is attached through the linker to an oligonucleotide that is hybridized to an oligonucleotide in the shell of oligonucleotides that is attached to the nanoparticle core. In some embodiments, the second antigen is attached to the external surface of the nanoparticle core through a linker. In some embodiments, the second antigen is encapsulated in the nanoparticle core. In further embodiments, the first antigen is attached to one or more oligonucleotides in the shell of oligonucleotides through a linker. In some embodiments, the first antigen is attached through the linker to an oligonucleotide in the shell of oligonucleotides that is attached to the nanoparticle core. In further embodiments, the first antigen is attached through the linker to an oligonucleotide that is hybridized to an oligonucleotide in the shell of oligonucleotides that is attached to the nanoparticle core. In yet further embodiments, the first antigen is attached to the external surface of the nanoparticle core through a linker. In some embodiments, a SNA of the disclosure comprises a third antigen that is a major histocompatibility complex type I (MHC-I) antigen. In further embodiments, a SNA of the disclosure comprises a fourth antigen that is a major histocompatibility complex type II (MHC-II) antigen. In some embodiments, the third antigen is encapsulated in the nanoparticle core. In further embodiments, the fourth antigen is attached to one or more oligonucleotides in the shell of oligonucleotides through a linker. In still further embodiments, the fourth antigen is attached through the linker to an oligonucleotide in the shell of oligonucleotides that is attached to the nanoparticle core. In yet further embodiments, the fourth antigen is attached through the linker to an oligonucleotide that is hybridized to an oligonucleotide in the shell of oligonucleotides that is attached to the nanoparticle core. In some embodiments, the fourth antigen is attached to the external surface of the nanoparticle core through a linker. In some embodiments, the third antigen is attached to one or more oligonucleotides in the shell of oligonucleotides through a linker. In some embodiments, the third antigen is attached through the linker to an oligonucleotide in the shell of oligonucleotides that is attached to the nanoparticle core. In further embodiments, the third antigen is attached through the linker to an oligonucleotide that is hybridized to an oligonucleotide in the shell of oligonucleotides that is attached to the nanoparticle core. In still further embodiments, the third antigen is attached to the external surface of the nanoparticle core through a linker. In some embodiments, the fourth antigen is encapsulated in the nanoparticle core. In various embodiments, the first antigen and the third antigen are the same. In some embodiments, the first antigen and the third antigen are different. In some embodiments, the second antigen and the fourth antigen are the same. In further embodiments, the second antigen and the fourth antigen are different. In various embodiments, the MHC-I antigen is OVA257-264 (OVA1) (SEQ ID NO: 7), GP100 (25-33) (KVPRNQDWL (SEQ ID NO: 11)), TC-1 E6 (49-58) (VYDFAFRDLC (SEQ ID NO: 12)), TC-1 E7 (49-57) (RAHYNIVTF (SEQ ID NO: 13)), PSMA (634-642) (SAVKNFTEI (SEQ ID NO: 14)), SPAS-1 (SNC9-H8) (STHVNHLHC (SEQ ID NO: 15)), SIMS2 (237-245) (SLDLKLIFL (SEQ ID NO: 16)), PAP (115-123) (SAMTNLAAL (SEQ ID NO: 17)), B16 MART-1 (M27) (LCPGNKYEM (SEQ ID NO: 9)), TRP-1 (252-260) (ATGKNVCDV (SEQ ID NO: 18)), TRP-1 (252V260M) (ATGKNVCDM (SEQ ID NO: 19)), TRP-1 (455-463) (TAPDNLGYA (SEQ ID NO: 20)), TRP-1 (455A463M) (TAPDNLGYM (SEQ ID NO: 21)), TRP-2 (180-188) (SVYDFFVWL (SEQ ID NO: 22)), Melan-A/MART-(127-135), Tyrokinase(1-9), Tyrokinase(369-377D), MC38 Adpgk (ASMTNMELM (SEQ ID NO: 23)), Irgq-Minimum (AALLNSAVL (SEQ ID NO: 24)), Irgq-Long peptide (KARDETAALLNSAVLGAAPLFVPPAD (SEQ ID NO: 25)), or a combination thereof. In various embodiments, the MHC-II antigen is OVA323-339 (OVA2) (SEQ ID NO: 8), GP100: (46-58) (RQLYPEWTEAQRL (SEQ ID NO: 26)), TC-1 E6 (43-57) (QLLRREVYDFAFRDL (SEQ ID NO: 27)), SIMS2 (240-254) (LKLIFLDSRVTEVTG (SEQ ID NO: 28)), PAP (114-128) (MSAMTNLAALFPPEG (SEQ ID NO: 29)), B16 MART-1 (M30) (VDWENVSPELNSTDQ (SEQ ID NO: 30)), TRP-1 (113-127) (CRPGWRGAACNQKIL (SEQ ID NO: 31)), TRP-1 (106-130) (SGHNCGTCRPGWRGAACNQKILTVR (SEQ ID NO: 32)), Li-Key (77-92) (LRMKLPKPPKPVSQMR (SEQ ID NO: 33)), Tyrosinase (56-70), GP100 (44-59), GP100 (167-189), Melan-A/MART-1(102-111) (PAYEKLSAEQSPPPY (SEQ ID NO: 34)), Melan-A/MART-1(27-40) (AAGIGILTVILGVL (SEQ ID NO: 35)), Melan-A/MART-1(51-70) (RNGYRALMDKSLHVGTQCAL (SEQ ID NO: 36)), Melan-A/MART-1(51-73) (RNGYRALMDKSLHVGTQCALTRR (SEQ ID NO: 37)), Melan-A/MART-1(43-57) (IGCWYCRRRNGYRAL (SEQ ID NO: 38)), or a combination thereof. In some embodiments, at least one of the one or more immunostimulatory oligonucleotides is a toll-like receptor (TLR) agonist. In further embodiments, each of the one or more immunostimulatory oligonucleotides is a toll-like receptor (TLR) agonist. In still further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the TLR is TLR9. In some embodiments, the immunostimulatory oligonucleotide comprises a CpG nucleotide sequence. In further embodiments, one or more oligonucleotides in the shell of oligonucleotides comprises or consists of the sequence of 5′-TCCATGACGTTCCTGACGTT-3′ (SEQ ID NO: 39). In some embodiments, one or more oligonucleotides in the shell of oligonucleotides comprises or consists of the sequence of 5′-TCGTCGTTTTGTCGTTTTGTCGTT-3′ (SEQ ID NO: 40). In some embodiments, one or more oligonucleotides in the shell of oligonucleotides comprises or consists of the sequence of 5′-TCCATGACGTTCCTGACGTT(Spacer-18 (hexaethyleneglycol))₂Cholesterol-3′ (SEQ ID NO: 41). In further embodiments, one or more oligonucleotides in the shell of oligonucleotides comprises or consists of the sequence of 5′-TCGTCGTTTTGTCGTTTTGTCGTT(Spacer-18 (hexaethyleneglycol))₂Cholesterol-3′ (SEQ ID NO: 6). In various embodiments, at least about 10%, 20%, 30%, 40%, 50%, 60%, 70% 80%, 90%, 95%, or 100% of oligonucleotides in the shell of oligonucleotides is an immunostimulatory oligonucleotide. In various embodiments, the linker is a carbamate alkylene disulfide linker, a thiol linker, a disulfide linker, an amide alkylene disulfide linker, an amide alkylene thio-succinimidyl linker, or a combination thereof. In further embodiments, the nanoparticle core is a micelle, a liposome, a polymer, a lipid nanoparticle (LNP), or a combination thereof. In various embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), or chitosan. In some embodiments, the polymer is poly(lactic-co-glycolic acid) (PLGA). In some embodiments, the nanoparticle core is a liposome. In various embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol. In some embodiments, one or more oligonucleotides in the shell of oligonucleotides is attached to the external surface of the nanoparticle core through a lipid anchor group. In further embodiments, the lipid anchor group is attached to the 5′ end or the 3′ end of the one or more oligonucleotides. In still further embodiments, the lipid anchor group is tocopherol or cholesterol. In some embodiments, one or more oligonucleotides in the shell of oligonucleotides is modified on its 5′ end and/or 3′ end with dibenzocyclooctyl (DBCO). In further embodiments, one or more oligonucleotides in the shell of oligonucleotides is modified on its 5′ end and/or 3′ end with a thiol. In various embodiments, the shell of oligonucleotides comprises DNA oligonucleotides, RNA oligonucleotides, or a combination thereof. In some embodiments, the shell of oligonucleotides comprises DNA oligonucleotides and RNA oligonucleotides. In further embodiments, the shell of oligonucleotides comprises single-stranded DNA, double-stranded DNA, single-stranded RNA, double-stranded RNA, or a combination thereof. In some embodiments, one or more oligonucleotides in the shell of oligonucleotides is a modified oligonucleotide. In various embodiments, the shell of oligonucleotides comprises about 2 to about 200 oligonucleotides. In some embodiments, the shell of oligonucleotides comprises about 2 to about 100 oligonucleotides. In further embodiments, the shell of oligonucleotides comprises about 150 oligonucleotides. In still further embodiments, the shell of oligonucleotides comprises about 200 oligonucleotides. In various embodiments, the shell of oligonucleotides comprises about 10 to about 80 oligonucleotides. In some embodiments, the shell of oligonucleotides comprises about 75 oligonucleotides. In various embodiments, each oligonucleotide in the shell of oligonucleotides is about 5 to about 1000 nucleotides in length. In some embodiments, each oligonucleotide in the shell of oligonucleotides is about 10 to about 50 nucleotides in length. In some embodiments, each oligonucleotide in the shell of oligonucleotides is about 20 to about 30 nucleotides in length. In further embodiments, diameter of the SNA is about 1 nanometer (nm) to about 500 nm. In some embodiments, diameter of the SNA is less than or equal to about 80 nanometers. In some embodiments, diameter of the SNA is less than or equal to about 50 nanometers. In various embodiments, the shell of oligonucleotides comprises a targeting oligonucleotide, an inhibitory oligonucleotide, a non-targeting oligonucleotide, or a combination thereof. In further embodiments, the inhibitory oligonucleotide is an antisense oligonucleotide, small interfering RNA (siRNA), an aptamer, a short hairpin RNA (shRNA), a DNAzyme, or an aptazyme.

In some aspects, the disclosure provides a composition comprising a plurality of SNAs as described herein. In some embodiments, at least two SNAs in the plurality comprise a different nanoparticle core.

In some aspects, the disclosure provides a pharmaceutical formulation comprising a plurality of SNAs or a composition as described herein, and a pharmaceutically acceptable carrier or diluent.

In further aspects, the disclosure provides a vaccine comprising a SNA, composition, or pharmaceutical formulation of the disclosure. In some embodiments, the vaccine comprises an adjuvant or additional adjuvant.

In some aspects, the disclosure provides an antigenic composition comprising a SNA of the disclosure in a pharmaceutically acceptable carrier, diluent, stabilizer, or preservative, or a pharmaceutical formulation of the disclosure, wherein the antigenic composition is capable of generating an immune response including antibody generation, cytotoxic T cell activation, helper T cell activation, or a protective immune response in a subject. In some embodiments, the immune response includes an antibody response. In further embodiments, the antibody response is a neutralizing antibody response or a protective antibody response.

In some aspects, the disclosure provides a method of inhibiting expression of a gene product comprising hybridizing a polynucleotide encoding the gene product to an inhibitory oligonucleotide as described herein, wherein hybridizing between the polynucleotide and the inhibitory oligonucleotide occurs over a length of the polynucleotide with a degree of complementarity sufficient to inhibit expression of the gene product. In various embodiments, expression of the gene product is inhibited in vivo or in vitro.

In some aspects, a method of producing an immune response in a subject, comprising administering to the subject an effective amount of a SNA, composition, pharmaceutical formulation, vaccine, or antigenic composition, each as described herein, thereby producing an immune response in the subject. In some embodiments, the immune response includes an antibody response. In further embodiments, the antibody response is a total antigen-specific antibody response. In some embodiments, the antibody response is a neutralizing antibody response or a protective antibody response.

In some aspects, the disclosure provides a method of immunizing a subject against one or more antigens comprising administering to the subject an effective amount of a SNA, composition, pharmaceutical formulation, vaccine, or antigenic composition, each as described herein, thereby immunizing the subject against the one or more antigens. In some embodiments, the composition or the vaccine is a cancer vaccine.

In some aspects, the disclosure provides a method of treating a cancer comprising administering to a subject an effective amount of a SNA, composition, pharmaceutical formulation, vaccine, or antigenic composition, each as described herein, thereby treating the cancer in the subject. In various embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof. In some embodiments, the cancer is melanoma. In some embodiments, the cancer is colon cancer. In further embodiments, the cancer is lymphoma. In some embodiments, a method of the disclosure further comprises administering an additional agent. In various embodiments, the additional agent is an anti-programmed cell death protein 1 (PD-1) antibody, an anti-programmed death-ligand 1 (PD-L1) antibody, a cytotoxic T lymphocyte antigen 4 (CTLA-4) antibody, a T cell immunoglobulin and ITIM domain (TIGIT) antibody, or a combination thereof.

### BRIEF DESCRIPTION OF THE FIGURES

FIG. 1 shows the Spherical Nucleic Acid (SNA) structure incorporating two antigen classes. (left) In dual-antigen SNA (DA-SNA) 1, the MHC-I (killer CD8+ T cell) antigen is encapsulated in the core of the liposome while the MHC-II (helper CD4+ T cell) antigen is hybridized to the adjuvant DNA shell. (right) DA-SNA 2 is the inverse.

FIG. 2 depicts that the SNA structure has demonstrated enhanced properties and can be used as a tool in immunotherapy.

FIG. 3 depicts additional demonstrated enhanced properties of the SNA structure specifically relevant to its use as a tool in immunotherapy.

FIG. 4 depicts a SNA structure incorporating two classes of antigen defined in unique placements.

FIG. 5 depicts results of experiments in which different dual-antigen SNAs activated cytokine production in different T cells.

FIG. 6 depicts results of experiments in which expression of memory markers in T cells differed based on DA-SNA structure.

FIG. 7 depicts results of experiments showing that circulating antigen-specific immune cells differed based on dual antigen spherical nucleic acid (DA-SNA) treatment.

FIG. 8 depicts results of experiments showing that circulating immune cell memory profiles stem from DA-SNA treatment.

FIG. 9 shows that structural changes using antigens to activate both Helper and Cytotoxic T cells leads to differences in efficacy. DA-SNA 2 dramatically altered the vaccine potency and antitumor effect, stalling the tumor.

FIG. 10 is a cartoon showing that spherical nucleic acids (SNAs) allows for elucidation of the impact of structural arrangement on the immune response.

FIG. 11 shows that structural SNA changes altered tumor growth. C57BL6 female mice were injected subcutaneously with 500,000 EG.7-OVA tumor cells in the right flank. On day 3, mice began weekly treatment. Four total vaccines were administered and tumors were measured every 2-3 days. N=9 per group. Statistics were calculated using a one-way ANOVA on day 24 followed by a Tukey multiple comparisons post-test *p<0.05; **p<0.01. Relative to FIG. 9, FIG. 11 additionally includes data showing the added benefit provided by the SNAs comprising both an MHC-I and MHC-II antigen, versus just comprising the MHC-I antigen. The presence of the OVA1 antigen without the OVA2 antigen (“OVA1 only SNA”) did not stall tumor growth for the extended period of time that DA-SNA 2 did.

FIG. 12 shows that immune cell spleen populations change as a result of vaccination. After four total weekly injections with SNAs in EG7.OVA tumor-bearing C57BL6 female mice, spleens were harvested and processed to identify cell populations. The percentage of the population that are (left) CD8+ (cytotoxic) T cells or (right) the relative ratio of CD8 to CD4 (helper) T cells is shown. Statistics were calculated using a 1-way ANOVA on day 24 followed by a Tukey multiple comparisons test. *p<0.05; **p<0.01; ***p<0.001; ****p<0.0001.

FIG. 13 shows that simultaneous DC activation with MHC I and II antigens leads to increased T cell proliferation. Using the OVA system with identified helper (OVA2) and cytotoxic (OVA1) antigens, enhanced immune activation was observed when both classes of antigen were delivered to the same cell in one SNA. Delivery of different antigen types to the same cell (Combined) elicited an approximate 2-fold enhancement in T cell proliferation compared to delivery of each antigen type on separate SNAs (Separate). *p<0.05;**p<0.01.

FIG. 14 shows that gene expression profiles of both CD8⁺ and CD4⁺ T Cells are unique based on vaccination condition. Principal component analysis (PCA) for transcriptomes of CD8 and CD4 T cell populations that were isolated from splenocytes following in vivo vaccinations with different treatments. PCA reduces the dimensionality of the genome data set consisting of a large number of interrelated variables while retaining as much as possible of the variation present in the data set. The grouping of the like colors (treatments) indicates that treatments were causing similar gene-level changes. The most separation on the x axis (PC1) indicates groups that were most dissimilar on the genetic level (i.e., left, admix and SNA containing OVA1H only, and right, admix and all three SNA groups). The next level of variance is the separation between groups on the y-axis (PC2).

FIG. 15 shows that gene expression profiles of CD8+ T cells differed based on vaccination condition. Gene expression heat maps for CD8+ T cell populations displayed uniquely activated gene pathways based on vaccination treatment conditions. Black boxes highlight genes with substantial variability across treatment groups.

FIG. 16 shows that gene expression profiles of CD4+ T cells differed based on vaccination condition. Gene expression heat maps for CD4+ T cell populations displayed uniquely activated gene pathways based on vaccination treatment conditions. Black boxes highlight genes with substantial variability across treatment groups.

FIG. 17 shows that DA-SNA 2 (see, e.g., FIG. 4 for description of DA-SNA-1 and DA-SNA-2) generated robust CD8⁺ T cell Memory. Antigen placement within dual-antigen SNAs (DA-SNAs) impacted immune responses. DA-SNA 2 enhanced the level of raised CD8⁺ effector function compared to other vaccination treatments. Both DA-SNAs enhanced CD4⁺ effector function.

FIG. 18 shows that DA-SNA 2 vaccination increased IFN-γ secretion upon antigen stimulation. Representative counts and images of IFN-γ-secreting splenic T cells upon different ex vivo stimulations.

FIG. 19 shows that DA-SNA 2 vaccination increased IFN-γ secretion upon antigen stimulation. Counts of IFN-γ-secreting splenic T cells upon different ex vivo stimulations. (representative images shown in FIG. 18).

FIG. 20 shows that IFN-γ and CD107a production increased in treatment group's CD8⁺ cytotoxic T cells. Intracellular IFN-γ (left and right) and CD107a (middle) levels in either CD8⁺ (left) or CD4⁺ (right) T cells demonstrate enhanced IFN-γ in T cells vaccinated with DA-SNA 2, upon different ex vivo stimulation. *P<0.05.

FIG. 21 shows that OVA1-specific T cell Memory was enhanced for SNA treatment. Tumor-bearing C57BL6 mice (n=6 per group) treated with different groups (as per same schedule as FIG. 11) were harvested at d17 for assessment of memory immune responses throughout the course of treatment. (left) The amount of CD8-antigen specific (OVA1) T cells, as measured by a dimer, is shown. (right) Of those specific T cells, the effector memory state is most enhanced for DA-SNA 2 immunized mice. Stars immediately above bars are comparisons against PBS group. Otherwise, specific comparisons are shown. *P<0.05; **p<0.01; ***p<0.001; ****p<0.0001.

FIG. 22 shows that OVA2-specific T cell Memory was enhanced for SNA treatment. Tumor-bearing C57BL6 mice (n=6 per group) treated with different groups (as per same schedule as FIG. 21) were harvested at d17 for assessment of memory immune responses throughout the course of treatment. (left) The amount of CD4-antigen specific (OVA2) T cells, as measured by a tetramer, is shown. (right) Of those specific T cells, the effector memory state is most enhanced for DA-SNA 1 immunized mice, with a slightly lower level for DA-SNA2 and OVA1H only SNA groups. *P<0.05.

FIG. 23 shows that DA-SNAs stalled tumor growth when applied to clinically-relevant melanoma tumor. C57BL6 female mice were injected subcutaneously with 100,000 B16.F110 melanoma tumor cells in the right flank. On day 3, mice began weekly treatment. Four total vaccines were administered and tumors were measured every 2-3 days. N=6 per group. Statistics were calculated using a one-way ANOVA on day 20 followed by a Tukey multiple comparisons post-test *p<0.05; **p<0.01.

FIG. 24 shows that combination therapy with Anti-PD-1 enhanced structure-driven SNA anti-tumor properties. C57BL6 female mice were injected subcutaneously with 100,000 B16.F10 melanoma tumor cells in the right flank. On day 3, mice began weekly treatment. Four total vaccines were administered with Anti-PD-1 checkpoint inhibitor injected 3 and 6 days following vaccine injection. Tumors were measured every 2-3 days. N=6 per group. Statistics were calculated using a one-way ANOVA comparing groups to Anti-PD-1 followed by a Tukey multiple comparisons post-test *p<0.05; **p<0.01.

FIG. 25 shows that combination therapy also positively impacted animal survival. C57BL6 female mice were injected subcutaneously with 100,000 B16.F10 melanoma tumor cells in the right flank. On day 3, mice began weekly treatment. Four total vaccines were administered with Anti-PD-1 checkpoint inhibitor injected 3 and 6 days following vaccine injection. Tumors were measured every 2-3 days and animals were sacrificed when tumor sized reached 1500 mm\hat{ }3. N=6 per group. Statistics for tumor volumes were calculated using a one-way ANOVA comparing groups to Anti-PD-1 followed by a Tukey multiple comparisons post-test. Survival statistics were compared using a Gehan-Breslow-Wilcoxon test. *p<0.05; **p<0.01.

FIG. 26 shows that the delivery of two classes of antigen from spherical nucleic acid (SNA) vaccines alters how the antigens are processed in vitro. a) Dual-antigen SNA (DA-SNA) vaccines synthesized to alter the placement of MHC-I and MHC-II restricted antigens within the same nanoparticle structure. b) The delivery of the two antigen classes on either separate nanoparticles (dashed, separate) or a singular DA-SNA (solid, combined) does not affect the fold-change in the population of CD86 or CD80-expressing CD11c⁺ DCs. c) CD8⁺ T cells specific for the OVA1 antigen (left) or CD4⁺ T cells specific for the OVA2 antigen raised from a co-culture of treatment-pulsed DCs with naïve splenic T cells. (left) T cell versus Separate and Combined OVA1 Encapsulated & OVA2 Hybridized (P=0.0025 and 0.0016, respectively) and Separate and Combined OVA2 Encapsulated & OVA1 Hybridized (P=0.0017 and 0.0094, respectively). (right) T cell versus Combined OVA1 Encapsulated & OVA2 Hybridized (P=0.0268) and Combined OVA2 Encapsulated & OVA1 Hybridized (P=0.0285). d) Median fluorescence intensity (MFI) of CD69 activation marker signal within the population of antigen-specific CD8⁺ (left) or CD4⁺ (right) T cells. (right) T cell versus Combined OVA2 Encapsulated & OVA1 Hybridized (P=0.0047). e) Fold-change in T cell proliferation from OT1 splenocytes specific for the OVA1 antigen after co-culture with treatment-pulsed DCs. (left) Separate versus Combined OVA1 Encapsulated & OVA2 Hybridized (P=0.0036); (right) Separate versus Combined OVA2 Encapsulated & OVA1 Hybridized (P=0.0306). For all panels, mean±s.e.m. shown, along with statistical significance between relevant comparisons. Significance was calculated using a one-way ANOVA with Sidak's multiple comparisons test, with n=3-4 replicates per group. ns=non-significant; *p<0.05; **p<0.01.

FIG. 27 is a schematic depiction of separate SNA combinations administered as a parallel comparison to DA-SNA treatment to assess the impact of antigen distribution. Separate nanoparticles were prepared as single antigen hybridized on surface and single antigen encapsulated within liposomal core.

FIG. 28 shows an example standard curve from Peptide Assay used to quantify the amount of peptide encapsulated within the liposomes. Data points were fit to a linear regression which always had an R²>0.98.

FIG. 29 shows an example standard curve from Phosphatidylcholine (PC) Assay used to quantify the concentration of liposomes through analysis of the lipid content using a manufacturer supplied standard. Data points were fit to a linear regression which always had an R²>0.98.

FIG. 30 shows ESI of the four purified peptide-DNA conjugates used in Example 2: M27, M30, OVA1, and OVA2, with confirmed masses of those expected.

FIG. 31 shows Dynamic Light Scattering (DLS) of liposomes and SNA. Size shift between encapsulated or DOPC liposome to either DA-SNA 1 or 2 structures with statistical significance is shown, indicative of SNA formation. Data show mean±s.e.m. from five independent measurements. ***p<0.001; ****p<0.0001.

FIG. 32 shows the gating strategy for FIG. 26C to quantify the percentage of live CD19−splenic cells that are double positive for either CD8⁺ the OVA1-H-2k^{b} or CD4⁺ OVA2-H-2-la^{d}.

FIG. 33 shows that antigen placement within DA-SNAs impacted immune responses after immunization. a) Schedule of fortnightly immunization for C57BL/6 mice. Dose: 6 nmol each antigen; 6 nmol adjuvant. b) Change of CD8⁺ (left) or CD4⁺ (right) cell populations in the spleen after vaccination scheme. (left) Admix versus DA-SNA 2 (P=0.0414). c) Intracellular production of IFN-γ pro-inflammatory cytokine (left) or CD107a degranulation marker (middle) was assessed upon ex vivo restimulation with peptide antigen. Polyfunctional T cells (double-positive for both markers) were quantified (right). DA-SNA 2 significantly elevated production of all markers in CD8⁺ T cells, whereas differences were more subtle amongst production in CD4⁺ T cells, with both DA-SNAs observably elevating levels above mice immunized with an admix vaccine. (top) Admix versus DA-SNA 2 and DA-SNA 1 versus DA-SNA 2 (left: P=0.0102 and 0.0124; middle: P=0.0188 and 0.0266; right: P=0.01 and 0.0119, all respectively). d) Effector memory phenotype, measured through CD44⁺CD62L− markers, was increased by DA-SNA 2 for CD8⁺ T cells. CD4⁺ effector function was most elevated for DA-SNA 1 immunization. (left) Admix versus DA-SNA 2 and DA-SNA 1 versus DA-SNA 2 (P=0.0016 and 0.0380, respectively). e) Representative counts and images (left) of IFN-γ-secreting splenic T cells upon different ex vivo stimulations along with total spot forming cells (SFCs) measured by ELISpot assay (right). For OVA1 ex vivo stimulation, Admix versus DA-SNA 2 and DA-SNA 1 versus DA-SNA 2 (P=<0.0001 and 0.0047, respectively). For OVA2 ex vivo stimulation, Admix versus DA-SNA 2 and DA-SNA 1 versus DA-SNA 2 (P=<0.0001 and 0.0004, respectively). Mean±s.e.m. shown. n=3 mice per group. Statistical significance between relevant comparisons is shown. For all panels significance was calculated using a one-way ANOVA with Sidak's multiple comparisons test. *p<0.05; **p<0.01; ***p<0.001; ****p<0.0001.

FIG. 34 shows that immunization of mice with differently structured vaccines induced specific differences in gene expression among CD8⁺ and CD4⁺ T cells. a) Principal Component Analysis (PCA) plot from the full transcriptome of CD8⁺ (left) and CD4⁺ (right) T cells. b) Gene expression changes represented by subset of log-fold change (LFC) for CD8+(left) and CD4⁺ (right) T cell populations as a result of different treatments. c) Selection of significantly enriched pathways calculated using GSEA analysis. Colors of squares correspond to the enrichment score for each pathway as a result of different treatment for CD8⁺ (left) and CD4⁺ (right) T cells. d) Gene signatures for CD8⁺ (top) and CD4⁺ (bottom) T cells. Colors refer to normalized (z-scored) gene expression levels. Selection of relevant genes labeled. e) Volcano plots of CD8⁺ (left) and CD4⁺ (right) T cells between a pairwise comparison of DA-SNA 2 and DA-SNA 1. Red dots indicate significantly expressed genes; a positive LFC indicates an upregulation for DA-SNA 2 with respect to DA-SNA 1 whereas a negative LFC indicates a downregulation for DA-SNA 2 with respect to DA-SNA 1.

FIG. 35 shows DA-SNA immune activation for enhanced tumor suppression. a-c) C57BL/6 mice were subcutaneously inoculated with E.G7-OVA cells (5×10⁵) in the right flank and provided weekly immunizations beginning at day 3 for a total of four vaccinations (6 nmol adjuvant, 6 nmol of each antigen). Average tumor growth curves and animal survival is shown. Tumor volume at day 24 of DA-SNA 2 compared to DA-SNA 1 and Admix (P=0.0121 and P=0.0084, respectively). Animal survival comparing DA-SNA 1 versus DA-SNA 2 (P=0.0219); DA-SNA 2 versus PBS and Admix (P=0.0030 and P=0.0029, respectively). d) Tumor weights following treatment schedule depicted in a (at day 15). Tumor weight comparing PBS versus DA-SNA 1 and DA-SNA 2 (P=0.0064 and P=0.0034, respectively) e) Evaluation of immune CD8⁺ T cells in the spleen at the conclusion of the experiment (left). Ratio of CD8⁺/CD4⁺ T cells (right). T cells in spleen of DA-SNA 2 versus PBS (P=0.0297), Admix (P=0.0013), and DA-SNA 1 (P=0.0002). f-i) Flow cytometric analysis of PBMCs at day 15 isolated from tumor-bearing mice under the schedule depicted in a. f) CD8⁺ T cells specific for the OVA1 antigen. DA-SNA 2 versus Admix (P=0.0401). g) Effector memory CD8⁺ T cells (CD44⁺/CD62L−) within this antigen-specific T cell subset. DA-SNA 2 versus Admix (P=0.0020). h) CD4⁺ T cells specific for the OVA2 antigen. Admix versus DA-SNA 1 (P=0.0229) and DA-SNA 2 (P=0.0436). i) Effector memory CD4⁺ T cells (CD44⁺/CD62L−). DA-SNA 1 versus Admix (P=0.0001) and DA-SNA 2 (P=0.0012). The data show mean±s.e.m. from two independent experiments (each experiment n=7-9). For panels b, d-g, i, significance was calculated using a one-way ANOVA with Tukey's multiple comparisons test. Panel h used a Welch ANOVA followed by a Dunnett's multiple comparisons test due to significant differences between groups in standard deviation. Panel c was analyzed using a Log-rank test. *p<0.05; **p<0.01; ***p<0.001.

FIG. 36 shows E.G7-OVA tumor growth curves from individual animals per treatment growth. Average tumor growth values are depicted in FIG. 35B. Line drawn at day 21 for easy comparison between spider plots of different groups.

FIG. 37 shows gross images of E.G7-OVA tumors used to calculate tumor weights at day 15. Dashed white circle represents mice with no tumors present. Scale bar: 1 mm.

FIG. 38 shows tumor inhibition utilizing dual antigen immunotherapy with immune checkpoint inhibitors. a-b) C57BL/6 mice were subcutaneously inoculated with B16-F10 cells (10⁵) in the right flank and provided weekly subcutaneous immunizations beginning at day 3 for a total of four vaccinations (9 nmol adjuvant, 9 nmol of each antigen). Average tumor growth curves and animal survival is shown. c) CD8⁺ T cells specific for the M27 antigen and memory CD8⁺ T cell markers 44+/62− in isolated PBMCs. PBS versus DA-SNA 1 (P=0.0060) and DA-SNA 2 (P=0.0001). d-e) B16-F10 tumor-bearing mice receiving weekly subcutaneous immunizations of DA-SNAs combined with an anti-PD-1 immune checkpoint inhibitor administered intraperitoneally 3 and 6 days post DA-SNA immunization. Average tumor growth curves and animal survival is shown. Tumor growth comparing anti-PD-1 versus DA-SNA 2+anti-PD-1 at day 17 (P=0.0354), 20 (P=0.0319), and 22 (P=0.0475). Animal survival comparing DA-SNA 2+anti-PD-1 versus PBS (P<0.0001) and anti-PD-1 (P<0.0001). f-k) Flow cytometric analysis of PBMCs at day 17 isolated from tumor-bearing mice receiving the schedule indicated in d. f) Evaluation of circulating CD8⁺ T cells and g) total effector memory CD8⁺ T cells (CD44+/62L−). For f: DA-SNA 2+anti-PD-1 versus PBS (P=0.0001), anti-PD-1 (P=0.0009)), and DA-SNA 1+anti-PD-1 (P<0.0001). For g: DA-SNA 2+anti-PD-1 versus PBS (P=<0.0001), anti-PD-1 (P<0.0001), and DA-SNA 1+anti-PD-1 (P=0.0090). DA-SNA 1+anti-PD-1 versus PBS (P=0.0023) and anti-PD-1 (P=0.0203). h) M27-specific CD8⁺/CD19− T cells. DA-SNA 2+anti-PD-1 versus PBS (P=0.0093), anti-PD-1 (P=0.0138), and DA-SNA 1+anti-PD-1 (P=0.0052). i) Quantification of circulating CD4⁺ T cells and j) assessment of effector memory CD4⁺ T cells (CD44+/62L−). For i: DA-SNA 2+anti-PD-1 versus DA-SNA 1+anti-PD-1 (P=0.0372). For j: PBS versus DA-SNA 1+anti-PD-1 (P=0.0268) and DA-SNA 2+anti-PD-1 (P=0.0349); anti-PD-1 versus DA-SNA 1+anti-PD-1 (P=0.0067) and DA-SNA 2+anti-PD-1 (P=0.0089). k) M30-specific CD4⁺/CD19− T cells. The data show mean±s.e.m. from two independent experiments (n=9-15). For all panels except b and e, significance was calculated using a one-way ANOVA with Tukey's multiple comparisons test. Animal survival was analyzed using a Log-rank test. Asterisks in d indicate statistically significant differences between DA-SNA 2 and anti-PD-1 treatment group. n.s.=not significant; n.d=not detected; *p<0.05, **p<0.01, ***p<0.001 and **** p<0.0001.

FIG. 39 shows B16-F10 tumor growth curves from individual animals per treatment group. Average tumor growth values are depicted in FIG. 38D.

FIG. 40 is a cartoon depicting that vaccine design has to fight against a heterogenous tumor population. CD4⁺ T cells can effect an antitumour response in the absence of CD8⁺ T cells by secreting cytokines, such as interferon-γ (Mumberg et al, 1999; Qin and Blankenstein, 2000), or by activation and recruitment of effector cells such as macrophages and eosinophils (Greenberg, 1991; Hung et al, 1998). However, the main role of CD4⁺ T cells in the immune response to cancer is to prime CD8⁺ cells and maintain their proliferation.

FIG. 41 is a cartoon depicting that vaccine design has to fight against a heterogenous tumor population.

FIG. 42 illustrates that antigen targeting strategies raising additional immune cells improve vaccine effectiveness.

FIG. 43 is a cartoon showing that potent vaccine delivery stimulates multiple classes of cells.

FIG. 44 shows results of experiments in which a-b) C57BL/6 mice were subcutaneously inoculated with MC38 cells (5×10⁵) in the right flank and provided weekly immunizations beginning at day 3 for a total of four vaccinations (6 nmol adjuvant, 6 nmol of each antigen). Average tumor growth curves and animal survival is shown. c) Average tumor volume at day 24.

### DETAILED DESCRIPTION

In various aspects, the present disclosure provides Spherical Nucleic Acids (SNAs), nanostructures with a core surrounded by a dense radial presentation of oligonucleotides, that target multiple different classes of immune cells (e.g., T cells) through incorporation and discrete structural placement of different classes of antigens. Antigens can be incorporated in places including but not limited to: encapsulated in the nanoparticle core, anchored to the surface, or conjugated to a complementary oligonucleotide strand that is hybridized to the adjuvant oligonucleotide SNA shell. In various embodiments, multi-antigen targeting SNAs of the disclosure provide defined structural presentation of vaccine components to optimally activate multiple different types of immune cells for a synergistic immune response.

Spherical nucleic acids (SNAs) are nanomaterials that can improve the delivery and potency of vaccine components. Comprised of a nanoparticle core with a dense surface layer of radially-oriented oligonucleotides (e.g., unmethylated cytosine-phosphate-guanine (CpG) motif DNA, which agonizes toll-like receptor 9 (TLR9) in antigen-presenting cells (APCs)), SNAs are modular structures with several advantages over conventional vaccines. They have high affinity target binding, rapid cellular uptake without the need for transfection reagents, high biocompatibility, reduced nuclease degradation, and easy drainage to the lymph nodes upon subcutaneous injection (Cutler, J. I.; Auyeung, E.; Mirkin, C. A. Spherical Nucleic Acids. J. Am. Chem. Soc. 2012, 134 (3), 1376-1391; Banga, R. J.; Chernyak, N.; Narayan, S. P.; Nguyen, S. T.; Mirkin, C. A. Liposomal Spherical Nucleic Acids. J. Am. Chem. Soc. 2014, 136 (28), 9866-9869; Radovic-Moreno, A. F.; Chernyak, N.; Mader, C. C.; Nallagatla, S.; Kang, R. S.; Hao, L.; Walker, D. A.; Halo, T. L.; Merkel, T. J.; Rische, C. H.; Anantatmula, S.; Burkhart, M.; Mirkin, C. A.; Gryaznov, S. M. Immunomodulatory Spherical Nucleic Acids. Proc. Natl. Acad. Sci. 2015, 112 (13), 3892-3897; Choi, C. H. J.; Hao, L.; Narayan, S. P.; Auyeung, E.; Mirkin, C. A. Mechanism for the Endocytosis of Spherical Nucleic Acid Nanoparticle Conjugates. Proc. Natl. Acad. Sci. 2013, 110 (19), 7625-7630; Rosi, N. L. Oligonucleotide-Modified Gold Nanoparticles for Intracellular Gene Regulation. Science 2006, 312 (5776), 1027-1030; Wang, S.; Qin, L.; Yamankurt, G.; Skakuj, K.; Huang, Z.; Chen, P.-C.; Dominguez, D.; Lee, A.; Zhang, B.; Mirkin, C. A. Rational Vaccinology with Spherical Nucleic Acids. Proc. Natl. Acad. Sci. 2019, 116 (21), 10473-10481). Their ease of synthesis and inherent modularity enable the design of compositionally equivalent structures with varied arrangement of the two key components-oligonucleotide adjuvant (immune system activator) shell and antigen (immune system target). Structural modifications have shown that vaccine architecture affects function and produces distinct immune responses, which has been demonstrated in multiple tumor models, such as model ovalbumin (OVA) lymphoma tumors and HPV, as well as prostate cancer-relevant systems targeting prostate-specific membrane antigen (PSMA). However, thus far, antigens have been incorporated that activate only one class of immune cells: cytotoxic CD8⁺ T cells.

Due to a tumor's heterogeneity and ability to evolve and evade the immune system, it is important not only to generate an enhanced major histocompatibility complex I (MHC-I) targeting cytotoxic CD8⁺ T cell response, but also engage synergistic interactions. In particular, both CD8⁺ and helper CD4⁺ T cells are necessary for long-lasting tumor rejection (Ostroumov, D.; Fekete-Drimusz, N.; Saborowski, M.; Kühnel, F.; Woller, N. CD4 and CD8 T Lymphocyte Interplay in Controlling Tumor Growth. Cell. Mol. Life Sci. 2018, 75 (4), 689-713; Shankaran, V.; Ikeda, H.; Bruce, A. T.; White, J. M.; Swanson, P. E.; Old, L. J.; Schreiber, R. D. IFNγ and Lymphocytes Prevent Primary Tumour Development and Shape Tumour Immunogenicity. Nature 2001, 410 (6832), 1107-1111). Therefore, the present disclosure provides SNAs having enhanced vaccine efficacy by simultaneously using MHC-I and -II antigens to prime both CD8⁺ and CD4⁺ T cells. This is especially important in melanoma, where traditional treatments such as chemotherapy or radiation are less effective, because it has a high mutational burden and can thus easily evade the immune system. However, current clinical alternatives are either ineffective at raising broad responses capable of handling mutations, logistically difficult, or expensive, and none consider the impact of structure on the resulting immune response. SNAs can function as robust cancer vaccines by controlling the presentation of immunostimulatory cues and target multiple melanoma-associated antigens in an effort to lower the potential for tumor immune evasion. The present disclosure utilizes a rational vaccinology approach to improve vaccine potency by presenting multiple epitopes in a specific structural arrangement to stimulate both cytotoxic and helper T cells.

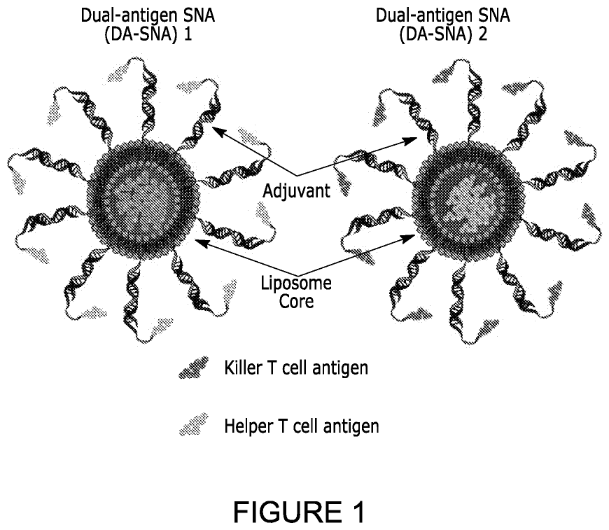

It is disclosed herein that the structural placement of these two classes of antigens (one targeting CD8⁺ and another targeting CD4⁺ T cells) dramatically alters the efficacy of the vaccine by inducing changes at the genetic, cellular, and organismal levels. Using model MHC-I and -II OVA antigens, it was observed that compositionally equivalent SNAs directed different immune responses. One particular SNA structure, with the MHC-I antigen hybridized to the shell and the MHC-II antigen encapsulated in the core, elicited a 2-fold increase in CD8⁺ effector cells compared to the inverse with swapped placement of the MHC-I and -II antigens (FIG. 1), and a 3-fold increase compared to a simple mixture of both antigens and adjuvant. Additionally, this SNA structure enhanced antigen-specific IFN-γ secretion and intracellular IFN-γ production to levels that were 5-fold higher than those from the inverse SNA or simple mixture. This was found to be a direct result of changes in the transcriptome, as measured by RNA-seq of the bulk CD8⁺ and CD4⁺ T cells in the spleen after immunization. Importantly, these trends translate to an in vivo melanoma system, where the different SNAs induce significant differences in tumor growth.

The present disclosure demonstrates how the heterogeneity of tumors can be addressed through the rational design of more complex vaccines. By incorporating this approach and considering antigen placement in vaccine design, it is demonstrated herein that an SNA vaccine's potency can be altered. The materials and methods of the disclosure are broadly applicable to the field, as it addresses the opportunity to use nanostructures to present and coordinate the processing of multiple immunostimulatory cues to immune cells. For example, the technology described herein is translatable to other systems and biological knowledge, as it informs the mechanistic understanding of the structural basis for vaccine function. The technology described herein allows for scalable, controllable, holistically generated immune responses. With an optimized structural presentation that incorporates multiple targets as described herein, there is a more robust response that, in the case of cancer, leads to faster and complete remission prior to tumor immune evasion.

As used in this specification and the appended claims, the singular forms “a,” “an,” and “the” include plural reference unless the context clearly dictates otherwise.

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a SNA to a subject in need of treatment with such an agent. Such modes include, but are not limited to, oral, topical, intravenous, intraarterial, intraperitoneal, intramuscular, intratumoral, intradermal, intranasal, and subcutaneous administration.

As used herein, “treating” and “treatment” refers to any reduction in the severity and/or onset of symptoms associated with a disease (e.g., cancer). Accordingly, “treating” and “treatment” includes therapeutic and prophylactic measures. One of ordinary skill in the art will appreciate that any degree of protection from, or amelioration of, the disease (e.g., cancer) is beneficial to a subject, such as a human patient. The quality of life of a patient is improved by reducing to any degree the severity of symptoms in a subject and/or delaying the appearance of symptoms.

As used herein, a “targeting oligonucleotide” is an oligonucleotide that directs a SNA to a particular tissue and/or to a particular cell type. In some embodiments, a targeting oligonucleotide is an aptamer. Thus, in some embodiments, a SNA of the disclosure comprises an aptamer attached to the exterior of the nanoparticle core, wherein the aptamer is designed to bind one or more receptors on the surface of a certain cell type.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist).

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme.

The term “non-targeting oligonucleotide” refers an oligonucleotide included, in some embodiments, in the shell of oligonucleotides of a SNA that is not associated with a particular activity (e.g., an immunostimulatory activity) but instead is used to achieve a certain density of oligonucleotides on the external surface of a SNA. Non-limiting examples of non-targeting oligonucleotides are an oligonucleotide comprising a scrambled nucleotide sequence and/or a homopolymeric oligonucleotide (e.g., a polythymidine oligonucleotide (such as T₂₀)).

An “antigenic composition” is a composition of matter suitable for administration to a human or animal subject (e.g., in an experimental or clinical setting) that is capable of eliciting a specific immune response, e.g., against an antigen, such as one or more of the antigens described herein. In the context of this disclosure, the term antigenic composition will be understood to encompass compositions that are intended for administration to a subject or population of subjects for the purpose of eliciting a protective or palliative immune response against an antigen, such as one or more of the antigens described herein.

The term “dose” as used herein refers to a measured portion of any of the SNAs of the disclosure (e.g., a SNA, antigenic composition, pharmaceutical formulation as described herein) taken by (administered to or received by) a subject at any one time.

An “immune response” is a response of a cell of the immune system, such as a B cell, T cell, or monocyte, to a stimulus, such as a SNA as described herein. An immune response can be a B cell response, which results in the production of specific antibodies, such as antigen specific neutralizing antibodies. An immune response can also be a T cell response, such as a CD4⁺ helper T cell response or a CD8⁺ cytotoxic T cell response. B cell and T cell responses are aspects of a “cellular” immune response. As described herein, an “immune response” can also be a “treatment based” response in which the immune system is being primed while actively fighting the tumor. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. Protective in this context does not necessarily require that the subject is completely protected against infection. A protective response is achieved when the subject is protected from developing symptoms of disease, or when the subject experiences a lower severity of symptoms of disease. A protective immune response can be measured, for example, by immune assays using a serum sample from an immunized subject and testing the ability of serum antibodies for inhibition of pseudoviral binding, such as: pseudovirus neutralization assay (or surrogate virus neutralization test), ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring B or T cell activation after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as a SNA as described herein, elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering, e.g., an antigenic composition of the disclosure increases the magnitude of an antigen-specific response, (e.g., by increasing the breadth of produced antibodies (i.e., in the case of administering a booster that primes the immune system against a variant), by increasing antibody titer and/or affinity, by increasing the frequency of antigen specific B or T cells, by inducing maturation effector function, or a combination thereof). The “maturity and memory” of B and T cells may also be measured as an indicator of an immune response.

“Adjuvant” refers to a substance which, when added to a composition comprising an antigen, nonspecifically enhances or potentiates an immune response to the antigen in the recipient upon exposure. In any of the aspects or embodiments of the disclosure, the SNAs provided herein comprise immunostimulatory oligonucleotides (for example and without limitation, a toll-like receptor (TLR) agonist) as adjuvants and comprise antigens as described herein. Additional adjuvants contemplated for use according to the disclosure include aluminum (e.g., aluminum hydroxide), lipid-based adjuvant AS01B, alum, MF59, in addition to TLR agonists as described herein (e.g., CpG DNA, TLR7's imiquimod, TLR8's Motolimod, TLR4's MPLA4, TLR3's Poly (I:C), or a combination thereof).

An “effective amount” or a “sufficient amount” of a substance is that amount necessary to effect beneficial or desired results, including clinical results, and, as such, an “effective amount” depends upon the context in which it is being applied. In the context of administering a SNA of the disclosure, for example, an effective amount contains sufficient antigen to elicit an immune response. In some embodiments, an effective amount of SNA is an amount sufficient to inhibit gene expression. An effective amount can be administered in one or more doses as described further herein. Efficacy can be shown in an experimental or clinical trial, for example, by comparing results achieved with a substance of interest compared to an experimental control.

All references, patents, and patent applications disclosed herein are incorporated by reference with respect to the subject matter for which each is cited, which in some cases may encompass the entirety of the document.

### Spherical Nucleic Acids

Spherical nucleic acids (SNAs) comprise densely functionalized and highly oriented polynucleotides on the surface of a nanoparticle core. In various aspects, the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen. In various embodiments, the nanoparticle core is a micelle, a liposome, a polymer, a lipid nanoparticle (LNP), or a combination thereof. In various embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), or chitosan. In some embodiments, the polymer is poly(lactic-co-glycolic acid) (PLGA). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

Liposomes are spherical, self-closed structures in a varying size range comprising one or several hydrophobic lipid bilayers with a hydrophilic core. The diameter of these lipid based carriers range from 0.15-1 micrometers, which is significantly higher than an effective therapeutic range of 20-100 nanometers. Liposomes termed small unilamellar vesicles (SUVs), can be synthesized in the 20-50 nanometer size range, but encounter challenges such as instability and aggregation leading to inter-particle fusion. This inter-particle fusion limits the use of SUVs in therapeutics. In some aspects, liposomal spherical nucleic acids (LSNAs) comprise a liposomal core, a shell of oligonucleotides attached to the external surface of the liposomal core, the shell of oligonucleotides comprising one or more immunostimulatory oligonucleotides; and a first antigen that is a major histocompatibility complex type I (MHC-I) antigen, and a second antigen that is a major histocompatibility complex type II (MHC-II) antigen. Antigens contemplated for use according to the disclosure are further described herein below.

Liposomal particles, for example as disclosed in International Patent Application No. PCT/US2014/068429 (incorporated by reference herein in its entirety) are therefore provided by the disclosure. Liposomal particles of the disclosure have at least a substantially spherical geometry, an internal side and an external side, and comprise a plurality of lipid groups. In various embodiments, the plurality of lipid groups comprises a lipid selected from the group consisting of the phosphatidylcholine, phosphatidylglycerol, and phosphatidylethanolamine families of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phospho-(1′-rac-glycerol) (DOPG), 1,2-distearoyl-sn-glycero-3-phosphoethanolamine (DSPE), cardiolipin, lipid A, monophosphoryl Lipid A (MPLA), or a combination thereof. In various embodiments, at least one oligonucleotide in the shell of oligonucleotides is attached to the exterior of the liposomal core through a lipid anchor group. In some embodiments, each oligonucleotide in the shell of oligonucleotides is attached to the exterior of the nanoparticle core through a lipid anchor group. In further embodiments, the lipid anchor group is attached to the 5′ end or the 3′ end of the at least one oligonucleotide. In still further embodiments, the lipid anchor group is tocopherol or cholesterol. Thus, in various embodiments, at least one of the oligonucleotides in the shell of oligonucleotides is an oligonucleotide-lipid conjugate containing a lipid anchor group, wherein said lipid anchor group is adsorbed into the lipid bilayer. In some embodiments, all of the oligonucleotides in the shell of oligonucleotides is an oligonucleotide-lipid conjugate containing a lipid anchor group, wherein said lipid anchor group is adsorbed into the lipid bilayer. In various embodiments, 10%, 20%, 30%, 40%, 50%, 60%, 70%, 80%, 90% or 100% of the oligonucleotides in the shell of oligonucleotides is attached (e.g., adsorbed) to the exterior of the liposomal core through a lipid anchor group. The lipid anchor group comprises, in various embodiments, tocopherol, palmitoyl, dipalmitoyl, stearyl, distearyl, or cholesterol. Methods of making oligonucleotides comprising a lipid anchor are disclosed herein. By way of example, first an oligonucleotide and phosphoramidite-modified-tocopherol are provided, and the oligonucleotide is then exposed to the phosphoramidite-modified-tocopherol to create the tocopherol modified oligonucleotide. While not meant to be limiting, any chemistry known to one of skill in the art can be used to attach the lipid anchor to the oligonucleotide, including amide linking or click chemistry.

Methods of making a liposomal SNA (LSNA) are described herein and are generally known (see, e.g., Wang et al., Proc. Natl. Acad. Sci. 2019, 116 (21), 10473-10481, incorporated by reference herein in its entirety).

Lipid nanoparticle spherical nucleic acids (LNP-SNAs) are comprised of a lipid nanoparticle core decorated with a shell of oligonucleotides. The lipid nanoparticle core comprises an ionizable lipid, a phospholipid, a sterol, a lipid-polyethylene glycol (lipid-PEG) conjugate, and a first antigen that is a major histocompatibility complex type I (MHC-l) antigen, and a second antigen that is a major histocompatibility complex type II (MHC-II) antigen. Antigens contemplated for use according to the disclosure are further described herein below. The shell of oligonucleotides is attached to the external surface of the lipid nanoparticle core, and in any of the aspects or embodiments of the disclosure the shell of oligonucleotides comprises one or more immunostimulatory oligonucleotides. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), monophosphoryl Lipid A (MPLA), or a combination thereof. In further embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (p-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

In any of the aspects or embodiments of the disclosure an oligonucleotide is attached to the exterior of a lipid nanoparticle core via a covalent attachment of the oligonucleotide to a lipid-polyethylene glycol (lipid-PEG) conjugate. In various embodiments, 10%, 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In various embodiments, one or more oligonucleotides in the oligonucleotide shell is attached (e.g., adsorbed) to the exterior of the lipid nanoparticle core through a lipid anchor group as described herein. In various embodiments, 10%, 20%, 30%, 40%, 50%, 60%, 70%, 80%, 90% or 100% of the oligonucleotides in the shell of oligonucleotides is attached (e.g., adsorbed) to the exterior of the lipid nanoparticle core through a lipid anchor group as described herein. The lipid anchor group is, in various embodiments, attached to the 5′- or 3′-end of the oligonucleotide. In various embodiments, the lipid anchor group is tocopherol, palmitoyl, dipalmitoyl, stearyl, distearyl, or cholesterol.

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of SNAs, each SNA comprising one or more oligonucleotides attached thereto. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the one or more oligonucleotides attached thereto.

### Antigens

Spherical nucleic acids (SNAs) of the disclosure comprise, in various aspects, (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen. As described herein, the number and positioning of the MHC-I and MHC-II antigens affect the potency of the SNA in its use as, e.g., a vaccine. MHC-I antigens prime CD8⁺ T cells, while MHC-II antigens prime CD4⁺ T cells. In any of the aspects or embodiments of the disclosure, a SNA comprises at least one MHC-I antigen and at least one MHC-II antigen. MHC-I antigens contemplated by the disclosure include, but are not limited to, OVA₂₅₇₋₂₆₄ (OVA1) (SEQ ID NO: 7), GP100 (25-33) (KVPRNQDWL (SEQ ID NO: 11)), TC-1 E6 (49-58) (VYDFAFRDLC (SEQ ID NO: 12)), TC-1 E7 (49-57) (RAHYNIVTF (SEQ ID NO: 13)), PSMA (634-642) (SAVKNFTEI (SEQ ID NO: 14)), SPAS-1 (SNC9-H8) (STHVNHLHC (SEQ ID NO: 15)), SIMS2 (237-245) (SLDLKLIFL (SEQ ID NO: 16)), PAP (115-123) (SAMTNLAAL (SEQ ID NO: 17)), B16 MART-1 (M27) (LCPGNKYEM (SEQ ID NO: 9)), TRP-1 (252-260) (ATGKNVCDV (SEQ ID NO: 18)), TRP-1 (252V260M) (ATGKNVCDM (SEQ ID NO: 19)), TRP-1 (455-463) (TAPDNLGYA (SEQ ID NO: 20)), TRP-1 (455A463M) (TAPDNLGYM (SEQ ID NO: 21)), TRP-2 (180-188) (SVYDFFVWL (SEQ ID NO: 22)), Melan-A/MART-(127-135), Tyrokinase(1-9), Tyrokinase(369-377D), MC38 Adpgk (ASMTNMELM (SEQ ID NO: 23)), Irgq-Minimum (AALLNSAVL (SEQ ID NO: 24)), Irgq-Long peptide (KARDETAALLNSAVLGAAPLFVPPAD (SEQ ID NO: 25)), or a combination thereof. MHC-II antigens contemplated by the disclosure include, but are not limited to, OVA₃₂₃₋₃₃₉ (OVA2) (SEQ ID NO: 8), GP100: (46-58) (RQLYPEWTEAQRL (SEQ ID NO: 26)), TC-1 E6 (43-57) (QLLRREVYDFAFRDL (SEQ ID NO: 27)), SIMS2 (240-254) (LKLIFLDSRVTEVTG (SEQ ID NO: 28)), PAP (114-128) (MSAMTNLAALFPPEG (SEQ ID NO: 29)), B16 MART-1 (M30) (VDWENVSPELNSTDQ (SEQ ID NO: 30)), TRP-1 (113-127) (CRPGWRGAACNQKIL (SEQ ID NO: 31)), TRP-1 (106-130) (SGHNCGTCRPGWRGAACNQKILTVR (SEQ ID NO: 32)), Li-Key (77-92) (LRMKLPKPPKPVSQMR (SEQ ID NO: 33)), Tyrosinase (56-70), GP100 (44-59), GP100 (167-189), Melan-A/MART-1(102-111) (PAYEKLSAEQSPPPY (SEQ ID NO: 34)), Melan-A/MART-1(27-40) (AAGIGILTVILGVL (SEQ ID NO: 35)), Melan-A/MART-1(51-70) (RNGYRALMDKSLHVGTQCAL (SEQ ID NO: 36)), Melan-A/MART-1(51-73) (RNGYRALMDKSLHVGTQCALTRR (SEQ ID NO: 37)), Melan-A/MART-1(43-57) (IGCWYCRRRNGYRAL (SEQ ID NO: 38)), or a combination thereof.

The disclosure contemplates that any configuration and combination of MHC-I and MHC-II antigens may be used in a SNA. Thus, in various aspects and embodiments, the disclosure provides SNAs in which a) both of the MHC-I and MHC-II antigens are encapsulated in the nanoparticle core and no antigen is associated with the external side of the nanoparticle core; b) both of the MHC-I and MHC-II antigens are encapsulated in the nanoparticle core and an MHC-I and/or MHC-II antigen is additionally associated with the external side of the nanoparticle core; c) both of the MHC-I and MHC-II antigens are on the external side of the nanoparticle core and no antigen is encapsulated in the nanoparticle core; d) both of the MHC-l and MHC-II antigens are on the external side of the nanoparticle core and an MHC-I and/or MHC-II antigen is additionally encapsulated in the nanoparticle core; e) the MHC-I antigen is encapsulated in the nanoparticle core and the MHC-II antigen is associated with the external side of the nanoparticle core; and f) the MHC-II antigen is encapsulated in the nanoparticle core and the MHC-I antigen is associated with the external side of the nanoparticle core. Accordingly, in various embodiments, the MHC-I antigen(s) and the MHC-II antigen(s) are encapsulated in the nanoparticle core, in association with the nanoparticle core on the external side of the nanoparticle core (through, e.g., covalent or non-covalent interactions), or any combination thereof. An antigen that is in association with the nanoparticle core on the external side of the nanoparticle core is, in any of the aspects or embodiments of the disclosure, located distal to the nanoparticle core. Thus, in some embodiments, an antigen is attached to the end of an oligonucleotide in the shell of oligonucleotides that is not attached to the nanoparticle core (e.g., if the oligonucleotide is attached to the nanoparticle core through its 3′ end, then the antigen is attached to the 5′ end of the oligonucleotide). Alternatively, in some embodiments, the antigen is attached to the end of an oligonucleotide in the shell of oligonucleotides that is attached to the nanoparticle core (e.g., if the oligonucleotide is attached to the nanoparticle core through its 3′ end, then the antigen is attached to the 3′ end of the oligonucleotide). In further embodiments, the antigen is attached to the end of an oligonucleotide that is proximal to the nanoparticle core, wherein the oligonucleotide is hybridized to an oligonucleotide that is attached to the nanoparticle core. Alternatively, in some embodiments the antigen is attached to the end of an oligonucleotide that is distal to the nanoparticle core, wherein the oligonucleotide is hybridized to an oligonucleotide that is attached to the nanoparticle core.

As described herein, in some aspects the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen. In some embodiments, the first antigen is encapsulated in the nanoparticle core. In further embodiments, the second antigen is attached to one or more oligonucleotides in the shell of oligonucleotides through a linker. As used herein, an antigen that is “attached to one or more oligonucleotides in the shell of oligonucleotides through a linker” may be attached in various ways, including but not limited to a) the antigen is attached directly to an oligonucleotide that is attached to the nanoparticle core; and/or b) the antigen is attached to an oligonucleotide that is hybridized to an oligonucleotide that is attached to the nanoparticle core. In any of the aspects or embodiments of the disclosure, when an antigen is attached directly to an oligonucleotide that is attached to the nanoparticle core, the antigen is attached to a non-targeting oligonucleotide. Thus, in some embodiments, the second antigen is attached through the linker to an oligonucleotide in the shell of oligonucleotides that is attached to the nanoparticle core. In further embodiments, the second antigen is attached through the linker to an oligonucleotide that is hybridized to an oligonucleotide in the shell of oligonucleotides that is attached to the nanoparticle core. In still further embodiments, the second antigen is attached to the external surface of the nanoparticle core through a linker. In some embodiments, the second antigen is encapsulated in the nanoparticle core. In further embodiments, the first antigen is attached to one or more oligonucleotides in the shell of oligonucleotides through a linker. In various embodiments, the first antigen is attached through the linker to an oligonucleotide in the shell of oligonucleotides that is attached to the nanoparticle core. In some embodiments, the first antigen is attached through the linker to an oligonucleotide that is hybridized to an oligonucleotide in the shell of oligonucleotides that is attached to the nanoparticle core. In further embodiments, the first antigen is attached to the external surface of the nanoparticle core through a linker. In some embodiments, a SNA comprises a third antigen that is a major histocompatibility complex type I (MHC-I) antigen. In further embodiments, a SNA comprises a fourth antigen that is a major histocompatibility complex type II (MHC-II) antigen. In some embodiments, the third antigen is encapsulated in the nanoparticle core. In further embodiments, the fourth antigen is attached to one or more oligonucleotides in the shell of oligonucleotides through a linker. In still further embodiments, the fourth antigen is attached through the linker to an oligonucleotide in the shell of oligonucleotides that is attached to the nanoparticle core. In some embodiments, the fourth antigen is attached through the linker to an oligonucleotide that is hybridized to an oligonucleotide in the shell of oligonucleotides that is attached to the nanoparticle core. In some embodiments, the fourth antigen is attached to the external surface of the nanoparticle core through a linker. In further embodiments, the third antigen is attached to one or more oligonucleotides in the shell of oligonucleotides through a linker. In some embodiments, the third antigen is attached through the linker to an oligonucleotide in the shell of oligonucleotides that is attached to the nanoparticle core. In further embodiments, the third antigen is attached through the linker to an oligonucleotide that is hybridized to an oligonucleotide in the shell of oligonucleotides that is attached to the nanoparticle core. In still further embodiments, the third antigen is attached to the external surface of the nanoparticle core through a linker. In some embodiments, the fourth antigen is encapsulated in the nanoparticle core. In various embodiments, the first antigen and the third antigen are the same. In some embodiments, the first antigen and the third antigen are different. In still further embodiments, the second antigen and the fourth antigen are the same. In some embodiments, the second antigen and the fourth antigen are different.

In some aspects, the disclosure provides a SNA comprising a plurality of first antigens that are MHC-I antigens, a plurality of second antigens that are MHC-II antigens, or both. The first antigens and/or the second antigens may be encapsulated in the nanoparticle core, associated with the nanoparticle core on the external side of the nanoparticle core, or a combination thereof. In some embodiments, each of the plurality of first antigens is the same. In further embodiments, the plurality of first antigens comprises any combination of first antigens as described herein. In some embodiments, each of the plurality of second antigens is the same. In further embodiments, the plurality of second antigens comprises any combination of second antigens as described herein. Thus, a SNA may comprise any combination of first antigens and second antigens as described herein.

In some aspects, the disclosure provides a SNA comprising (a) a nanoparticle core; (b) a shell of oligonucleotides attached to the external surface of the nanoparticle core, the shell of oligonucleotides comprising one or more immunostimulatory oligonucleotides; and (c) one or more MHC-I antigens that are encapsulated in the nanoparticle core, and one or more MHC-II antigens that are in association with the nanoparticle core on the external side of the nanoparticle core. In further aspects, the disclosure provides a SNA comprising (a) a nanoparticle core; (b) a shell of oligonucleotides attached to the external surface of the nanoparticle core, the shell of oligonucleotides comprising one or more immunostimulatory oligonucleotides; and (c) one or more MHC-II antigens that are encapsulated in the nanoparticle core, and one or more MHC-I antigens that are in association with the nanoparticle core on the external side of the nanoparticle core. In some embodiments, some of the one or more MHC-I antigens are encapsulated in the nanoparticle core and are also in association with the nanoparticle core on the external side of the nanoparticle core. In some embodiments, some of the one or more MHC-II antigens are encapsulated in the nanoparticle core and are also in association with the nanoparticle core on the external side of the nanoparticle core. In further embodiments, some of the one or more MHC-I antigens and some of the one or more MHC-II antigens are encapsulated in the nanoparticle core and are also in association with the nanoparticle core on the external side of the nanoparticle core. In various embodiments, the one or more MHC-I antigens are all the same, while in some embodiments, the one or more MHC-I antigens comprises any combination of MHC-I antigens as described herein. In various embodiments, the one or more MHC-II antigens are all the same, while in some embodiments, the one or more MHC-II antigens comprises any combination of MHC-II antigens as described herein.

In further aspects, the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; (c) a first antigen that is a major histocompatibility complex type I (MHC-l) antigen, wherein the first antigen is encapsulated in the nanoparticle core, attached to one or more oligonucleotides in the shell of oligonucleotides through a linker, attached to the external surface of the nanoparticle core through the linker, or a combination thereof, and (d) a second antigen that is a major histocompatibility complex type II (MHC-II) antigen, wherein the second antigen is encapsulated in the nanoparticle core, attached to one or more oligonucleotides in the shell of oligonucleotides through a linker, attached to the external surface of the nanoparticle core through the linker, or a combination thereof.

In further aspects, the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; (c) a first antigen that is a major histocompatibility complex type I (MHC-l) antigen, wherein the first antigen is encapsulated in the nanoparticle core, attached to one or more oligonucleotides in the shell of oligonucleotides through a linker, attached to the external surface of the nanoparticle core through the linker, or a combination thereof; (d) a second antigen that is a major histocompatibility complex type II (MHC-II) antigen, wherein the second antigen is encapsulated in the nanoparticle core, attached to one or more oligonucleotides in the shell of oligonucleotides through a linker, attached to the external surface of the nanoparticle core through the linker, or a combination thereof; and (e) a third antigen that is a major histocompatibility complex type I (MHC-l) antigen, wherein the third antigen is encapsulated in the nanoparticle core, attached to one or more oligonucleotides in the shell of oligonucleotides through a linker, attached to the external surface of the nanoparticle core through the linker, or a combination thereof.

In some aspects, the disclosure provides a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; (c) a first antigen that is a major histocompatibility complex type I (MHC-l) antigen, wherein the first antigen is encapsulated in the nanoparticle core, attached to one or more oligonucleotides in the shell of oligonucleotides through a linker, attached to the external surface of the nanoparticle core through the linker, or a combination thereof; (d) a second antigen that is a major histocompatibility complex type II (MHC-II) antigen, wherein the second antigen is encapsulated in the nanoparticle core, attached to one or more oligonucleotides in the shell of oligonucleotides through a linker, attached to the external surface of the nanoparticle core through the linker, or a combination thereof; and (e) a third antigen that is a major histocompatibility complex type I (MHC-I) antigen, wherein the third antigen is encapsulated in the nanoparticle core, attached to one or more oligonucleotides in the shell of oligonucleotides through a linker, attached to the external surface of the nanoparticle core through the linker, or a combination thereof; and (f) a fourth antigen that is a major histocompatibility complex type II (MHC-II) antigen, wherein the fourth antigen is encapsulated in the nanoparticle core, attached to one or more oligonucleotides in the shell of oligonucleotides through a linker, attached to the external surface of the nanoparticle core through the linker, or a combination thereof.

In any of the aspects or embodiments of the disclosure, a spherical nucleic acid (SNA) comprises: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen, wherein the first antigen is encapsulated in the nanoparticle core, and wherein the second antigen is attached through the linker to an oligonucleotide that is hybridized to an oligonucleotide in the shell of oligonucleotides that is attached to the nanoparticle core. In any of the aspects or embodiments of the disclosure, a method as described herein comprises administering to a subject a spherical nucleic acid (SNA) comprising: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen, wherein the first antigen is encapsulated in the nanoparticle core, and wherein the second antigen is attached through the linker to an oligonucleotide that is hybridized to an oligonucleotide in the shell of oligonucleotides that is attached to the nanoparticle core.

In any of the aspects or embodiments of the disclosure, a spherical nucleic acid (SNA) comprises: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen, wherein the second antigen is encapsulated in the nanoparticle core, and wherein the first antigen is attached through the linker to an oligonucleotide that is hybridized to an oligonucleotide in the shell of oligonucleotides that is attached to the nanoparticle core. In any of the aspects or embodiments of the disclosure, a method as described herein comprises administering to a subject a spherical nucleic acid (SNA) comprises: (a) a nanoparticle core; (b) a shell of oligonucleotides attached to the external surface of the nanoparticle core, the shell of oligonucleotides comprising one or more immunostimulatory oligonucleotides; and (c) a first antigen that is a major histocompatibility complex type I (MHC-I) antigen, and a second antigen that is a major histocompatibility complex type II (MHC-II) antigen, wherein the second antigen is encapsulated in the nanoparticle core, and wherein the first antigen is attached through the linker to an oligonucleotide that is hybridized to an oligonucleotide in the shell of oligonucleotides that is attached to the nanoparticle core.

### Oligonucleotides

The disclosure provides spherical nucleic acids (SNAs) comprising a nanoparticle core, a shell of oligonucleotides attached to the exterior of the nanoparticle core, the shell of oligonucleotides comprising one or more immunostimulatory oligonucleotides, and a first antigen that is a major histocompatibility complex type I (MHC-I) antigen, and a second antigen that is a major histocompatibility complex type II (MHC-II) antigen. In various embodiments, about or at least about 5%, 10%, 20%, 30%, 40%, 50%, 60%, 70% 80%, 90%, 95%, or 100% of oligonucleotides in the shell of oligonucleotides are immunostimulatory oligonucleotides. In various embodiments, the shell of oligonucleotides comprises an inhibitory oligonucleotide, a targeting oligonucleotide, a non-targeting oligonucleotide, or a combination thereof. Oligonucleotides contemplated for use according to the disclosure include those attached to a nanoparticle core through any means (e.g., covalent or non-covalent attachment). Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e., a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C, to Co alkyl or C2 to Co alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C, to Co lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 1000 nucleotides in length. More specifically, an oligonucleotide of the disclosure is about about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. For example, and without limitation, in some embodiments the shell of oligonucleotides comprises a plurality of immunostimulatory oligonucleotides, wherein one immunostimulatory oligonucleotide has a sequence that is different than at least one other immunostimulatory oligonucleotide in the plurality.

In some embodiments, one or more oligonucleotides in the shell of oligonucleotides comprises or consists of a (GGX)ₙ nucleotide sequence, wherein n is 2-20 and X is a nucleobase (A, C, T, G, or U). In some embodiments, the (GGX)ₙ nucleotide sequence is on the 5′ end of the one or more oligonucleotides. In some embodiments, the (GGX)ₙ nucleotide sequence is on the 3′ end of the one or more oligonucleotides. In some embodiments, one or more oligonucleotides in the shell of oligonucleotides comprises or consists of a (GGT)ₙ nucleotide sequence, wherein n is 2-20. In some embodiments, the (GGT)ₙ nucleotide sequence is on the 5′ end of the one or more oligonucleotides. In some embodiments, the (GGT)ₙ nucleotide sequence is on the 3′ end of the one or more oligonucleotides.

In some embodiments, an oligonucleotide in the shell of oligonucleotides is a targeting oligonucleotide, such as an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Spacers. In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 0.5 pmol/cm² will be adequate to provide a stable SNA. In further embodiments, a surface density of oligonucleotides that is at least about 1 pmol/cm², 1.5 pmol/cm², or 2 pmoles/cm² will be adequate to provide a stable SNA (e.g., LSNA or LNP-SNA). In some aspects, the surface density of a SNA of the disclosure is at least 15 pmoles/cm². Methods are also provided wherein the oligonucleotide is attached to the nanoparticle core of the SNA at a surface density of about 0.5 pmol/cm² to about 1000 pmol/cm², or about 2 pmol/cm² to about 200 pmol/cm², or about 10 pmol/cm² to about 100 pmol/cm². In some embodiments, the surface density is about 1.7 pmol/cm². In some embodiments, the surface density is about 2 pmol/cm². In further embodiments, the surface density is at least about 0.5 pmol/cm², at least about 0.6 pmol/cm², at least about 0.7 pmol/cm², at least about 0.8 pmol/cm², at least about 0.9 pmol/cm², at least about 1 pmol/cm², at least about 1.5 pmol/cm², at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², less than about 50 pmol/cm², less than about 55 pmol/cm², less than about 60 pmol/cm², less than about 65 pmol/cm², less than about 70 pmol/cm², less than about 75 pmol/cm², less than about t 80 pmol/cm², less than about 85 pmol/cm², less than about 90 pmol/cm², less than about 95 pmol/cm², less than about 100 pmol/cm², less than about 125 pmol/cm², less than about 150 pmol/cm², less than about 175 pmol/cm², less than about 200 pmol/cm², less than about 250 pmol/cm², less than about 300 pmol/cm², less than about 350 pmol/cm², less than about 400 pmol/cm², less than about 450 pmol/cm², less than about 500 pmol/cm², less than about 550 pmol/cm², less than about 600 pmol/cm², less than about 650 pmol/cm², less than about 700 pmol/cm², less than about 750 pmol/cm², less than about 800 pmol/cm², less than about 850 pmol/cm², less than about 900 pmol/cm², less than about 950 pmol/cm², or less than about 1000 pmol/cm².

Alternatively, the density of oligonucleotide attached to the SNA is measured by the number of oligonucleotides attached to the SNA. With respect to the surface density of oligonucleotides attached to a SNA of the disclosure, it is contemplated that a SNA as described herein comprises or consists of about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20, or about 75 to about 200, or about 75 to about 150, or about 100 to about 200, or about 150 to about 200 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155,160, 165, 170, 175, 180, 185, 190, 195, or 200 oligonucleotides in the shell of oligonucleotides attached to the nanoparticle core. In further embodiments, a SNA consists of 5, 10, 20, 30, 40, 45, 50, 55, 60, 65, 70, 75, 80, 85, 90, 95, 100, 105, 110, 115, 120, 125, 130, 135, 140,145, 150, 155, 160, 165, 170, 175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20, 50, 60, 70, 75, 80, 90, 100, 150, 160, 170, 175, 180, 190, 200, or more oligonucleotides. In some embodiments, the shell of oligonucleotides attached to the nanoparticle core of the SNA consists of 2, 3, 4, 5, 6, 7, 8, 9, 10, 11, 12, 13, 14, 15, 16, 17, 18, 19, 20, 30, 40, 50, 60, 70, 75, 80, 90, 100, 150, 160, 170, 175, 180, 190, or 200 oligonucleotides. In some embodiments, the shell of oligonucleotides comprises about 10 to about 80 oligonucleotides. In some embodiments, the shell of oligonucleotides comprises or consists of about 75 oligonucleotides.

Linkers. The disclosure provides compositions and methods in which an antigen is associated with and/or attached to the surface of a SNA via a linker. The linker can be, in various embodiments, a cleavable linker, a non-cleavable linker, a traceless linker, and a combination thereof.

The linker links the antigen to the oligonucleotide in the disclosed SNA or links the antigen to the surface of the SNA (i.e., Antigen-LINKER-Oligonucleotide or Antigen-LINKER). The oligonucleotide can be hybridized to another oligonucleotide attached to the SNA or can be directly attached to the SNA (e.g., via a lipid anchor group). Some specifically contemplated linkers include carbamate alkylene, carbamate alkylenearyl disulfide linkers, amide alkylene disulfide linkers, amide alkylenearyl disulfide linkers, thiol linkers, and amide alkylene succinimidyl linkers. In some cases, the linker comprises —NH—C(O)—O—C₂₋₅alkylene-S—S—C₂₋₇alkylene- or —NH—C(O)—C₂₋₅alkylene-S—S—C₂₋₇alkylene-. The carbon alpha to the —S—S— moiety can be branched, e.g., —CHX—S—S— or —S—S—CHY— or a combination thereof, where X and Y are independently Me, Et, or iPr. The carbon alpha to the antigen can be branched, e.g., —CHX—C₂₋₄alkylene-S—S—, where X is Me, Et, or iPr. In some cases, the linker is —NH—C(O)—O—CH₂—Ar—S—S—C₂₋₇alkylene-, and Ar is a meta- or para-substituted phenyl. In some cases, the linker is —NH—C(O)—C₂₋₄alkylene-N-succinimidyl-S—C₂₋₆alkylene-.

Additional linkers include an SH linker, SM linker, SE linker, and SI linker. The disclosure contemplates multiple points of attachment available for modulating antigen release (e.g., disulfide cleavage, linker cyclization, and dehybridization), and the kinetics of antigen release at each attachment point can be controlled. For example, steric bulk about the disulfide can decrease the rate of the S_{N}2 reaction; increased length of an alkyl spacer or steric bulk attached to the alkyl spacer can affect the rate of ring closure; and mismatched nucleotide sequences lower the melting temperature (Tₘ), while locked nucleic acids increase the Tₘ.

### Uses of SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 8 and TLR 9 that respond to specific oligonucleotides are located inside special intracellular compartments, called endosomes. The mechanism of modulation of, for example and without limitation, TLR 8 and TLR 9 receptors, is based on DNA-protein interactions.

As described herein, synthetic immunostimulatory oligonucleotides that contain CpG motifs that are similar to those found in bacterial DNA stimulate a similar response of the TLR receptors. Thus, CpG oligonucleotides of the disclosure have the ability to function as TLR agonists. Other TLR agonists contemplated by the disclosure include, without limitation, single-stranded RNA and small molecules (e.g., R848 (Resiquimod)). Therefore, immunomodulatory (e.g., immunostimulatory) oligonucleotides have various potential therapeutic uses, including treatment of diseases (e.g., cancer).

Accordingly, in some embodiments, methods of utilizing SNAs as described herein for modulating toll-like receptors are disclosed. The method up-regulates the Toll-like-receptor activity through the use of a TLR agonist. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Methods of Inducing an Immune Response

The disclosure also includes methods for eliciting an immune response in a subject in need thereof, comprising administering to the subject an effective amount of a SNA (e.g., formulated as an antigenic composition) of the disclosure. In various embodiments, administering SNAs of the disclosure (e.g., formulated as a composition, pharmaceutical formulation, or antigenic composition) to a subject results in an increase in the amount of neutralizing antibodies against the antigen(s) that is produced in the subject relative to the amount of neutralizing antibodies against the antigen(s) that is produced in a subject who was not administered the SNAs. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase.

In further embodiments, SNAs of the disclosure activate human peripheral blood mononuclear cells and generate an antibody response against one or more antigens as described herein. In some embodiments, the antibody response is a total antigen-specific antibody response. In further embodiments, administering SNAs of the disclosure (e.g., formulated as a composition, pharmaceutical formulation, or antigenic composition) to a subject results in an increase in the amount of total antigen-specific antibodies against the antigen(s) that is produced in the subject relative to the amount of total antigen-specific antibodies against the antigen(s) that is produced in a subject who was not administered the SNAs. In further embodiments, the increase is a 2-fold increase, a 5-fold increase, a 10-fold increase, a 50-fold increase, a 100-fold increase, a 200-fold increase, a 500-fold increase, a 700-fold increase, or a 1000-fold increase. A “total antigen-specific antibody response” is a measure of all of the antibodies (including neutralizing and non-neutralizing antibodies) that bind to a particular antigen.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4⁺, CD8⁺. The immune response generated by the SNA as disclosed herein generates an immune response and preferably treats a disease (e.g., cancer) as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In any of the aspects or embodiments of the disclosure, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, or a subcutaneous prime and a subcutaneous boost. Administration of more than one dose (typically two doses) is particularly useful in immunologically naive subjects or subjects of a hyporesponsive population (e.g., diabetics, or subjects with chronic kidney disease (e.g., dialysis patients)). In various embodiments, the second dose is administered about or at least about 2 weeks after the first dose. Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). In some embodiments, multiple doses are administered from one, two, three, four or five months apart. Antigenic compositions of the present disclosure may be administered to patients at substantially the same time as (e.g., during the same medical consultation or visit to a healthcare professional) other vaccines.

### Uses of LNP-SNAs to Treat a Disorder

In some aspects, a SNA of the disclosure is used to treat a disorder. Thus, in some aspects, the disclosure provides methods of treating a disorder comprising administering an effective amount of a SNA of the disclosure to a subject (e.g., a human subject) in need thereof, wherein the administering treats the disorder. In some aspects, the disclosure provides methods of treating a cancer comprising administering to a subject (e.g., a human subject) an effective amount of a SNA of the disclosure, thereby treating the cancer in the subject. In various embodiments, the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

### Additional Agents

In any of the aspects or embodiments of the disclosure, an additional agent is administered separately from a SNA of the disclosure. Thus, in various embodiments, a therapeutic agent is administered before, after, or concurrently with a SNA of the disclosure to treat a disorder (e.g., cancer).

In some aspects, the SNAs provided herein optionally further comprise an additional agent, or a plurality thereof. The additional agent is, in various embodiments, simply associated with an oligonucleotide in the shell of oligonucleotides attached to the exterior of the nanoparticle core of the SNA, and/or the additional agent is associated with the nanoparticle core of the SNA, and/or the additional agent is encapsulated in the nanoparticle core of the SNA. In some embodiments, the additional agent is associated with the end of an oligonucleotide in the shell of oligonucleotides that is not attached to the nanoparticle core (e.g., if the oligonucleotide is attached to the nanoparticle core through its 3′ end, then the additional agent is associated with the 5′ end of the oligonucleotide). Alternatively, in some embodiments, the additional agent is associated with the end of an oligonucleotide in the shell of oligonucleotides that is attached to the nanoparticle core (e.g., if the oligonucleotide is attached to the nanoparticle core through its 3′ end, then the additional agent is associated with the 3′ end of the oligonucleotide). In some embodiments, the additional agent is covalently associated with an oligonucleotide in the shell of oligonucleotides that is attached to the exterior of the nanoparticle core of the SNA. In some embodiments, the additional agent is non-covalently associated with an oligonucleotide in the shell of oligonucleotides that is attached to the exterior of the nanoparticle core of the SNA. However, it is understood that the disclosure provides SNAs wherein one or more additional agents are both covalently and non-covalently associated with oligonucleotides in the shell of oligonucleotides that is attached to the exterior of the lipid nanoparticle core of the SNA. It will also be understood that non-covalent associations include hybridization, protein binding, and/or hydrophobic interactions.

Additional agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof. In some embodiments, the additional agent is an anti-programmed cell death protein 1 (PD-1) antibody.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### Uses of SNAs in Gene Regulation

In some aspects of the disclosure, an oligonucleotide associated with a SNA of the disclosure (e.g., LNP-SNA, LSNA) inhibits the expression of a gene. Thus, in some embodiments, a SNA performs both a vaccine function and a gene inhibitory function. In such aspects, the shell of oligonucleotides that is attached to the external surface of the nanoparticle core comprises one or more immunostimulatory oligonucleotides and one or more inhibitory oligonucleotides designed to inhibit target gene expression.

Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an inhibitory oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

The following examples are given merely to illustrate the present disclosure and not in any way to limit its scope.

### EXAMPLES

### Example 1

Methods of synthesizing SNAs of the disclosure. In some embodiments, multi-antigen targeting SNA vaccines are synthesized using a liposome core. Lipid films containing DOPC (1,2-dioleoyl-sn-glycero-3-phosphocholine) are hydrated with PBS containing dissolved peptide antigens. These are then extruded, dialyzed overnight to remove excess unencapsulated peptide, and characterized using DLS, a pierce assay (for peptide quantification), and a PC assay (for lipid quantification). Separately, peptide conjugated complementary DNA (to the adjuvant) is synthesized. This process can take many chemistries but involves disulfide interactions between the peptide (method 1-cysteine or method 2-linker attached) and the complementary DNA (through thiol-termination). In the case of a cysteine (method 1), first the peptide is activated with Aldrithiol at room temperature for at least 1 h. This product is then mixed overnight with thiol-terminated complementary DNA at a 5:1 molar ratio. In method 2, the N-terminus of peptide on resin is reacted overnight at room temperature with an NHS ester terminated linker. This product is cleaved off the resin, deprotected, and purified prior to conjugation with thiol-terminated complementary DNA at a 10:1 molar ratio. In either method, final product is purified through polyacrylamide gel electrophoresis and analyzed using ESI. Purified product is mixed 1:1 with cholesterol-terminated DNA adjuvant and lyophilized. This is resuspended in duplex buffer and slow cooled to duplex (70 C 10 min, 25 C 1.5h, overnight at 4 C). Duplex is then mixed in an equimolar ratio with peptide encapsulated in the liposomes and the liposome is backfilled (up to 75 strands per liposome) to form SNAs.

### Example 2

Cancer vaccines must activate multiple immune cell types to be effective against aggressive tumors, but design considerations for these multi-targeted vaccines are underexplored. Herein, the impact of structural presentation of multiple antigenic peptides on immune responses at transcriptomic, cellular, and organismal levels is demonstrated. Spherical nucleic acid (SNA) nanoconstructs were used to investigate differences in antigen processing, cytokine production, and memory stemming from spatial distribution and nanoscale placement of two antigen classes to activate two T cell types. A single dual-antigen SNA (DA-SNA), compared to two single-antigen SNAs, elicited a 30% and two-fold increase in antigen-specific T cell activation and proliferation, respectively. Antigen placement within DA-SNAs changed immunological gene expression and tumor growth: encapsulating helper and externally-conjugating cytotoxic T cell antigens (termed DA-SNA 2) elevated antitumor gene pathways, stalling tumors in mice with lymphoma. When combined with anti-PD-1 checkpoint inhibitor in clinically relevant melanoma, DA-SNA 2 suppressed tumors and increased circulating T cell memory. This Example demonstrates the importance of implementing structural control afforded by modular nanoscale architectures to synthesize multi-antigen vaccines with improved efficacy.

Vaccination is an attractive strategy against cancers expressing targetable tumor-associated antigens and neoantigens. For melanoma, increasing efforts have been made to develop vaccines targeting identified tumor-associated proteins (e.g., gp100, MAGE-A3, MART-1, NY-ESO-1).¹⁻⁴ However, while these vaccines elicit some benefits (i.e., increasing activated melanoma-specific T cells), many are designed to activate only one immune cell type. Tumors can have significant heterogeneity and high mutational burdens^{5,6} that allow for easy escape of immune surveillance.⁷ Thus, vaccines that activate primarily one immune cell type are inadequate, necessitating vaccines containing antigens targeting multiple cell types to induce complete tumor remission.

Common approaches to elicit a multi-faceted immune response are administration of: 1) “long peptides” whose sequence covers multiple epitopes to activate both cytotoxic and helper T cells, or 2) multiple “minimal” peptide antigens each unique to T cell subclasses.⁸⁻¹¹ However, many of these ongoing efforts involve pools of peptides, with or without an adjuvant, delivered in saline as a mixture.

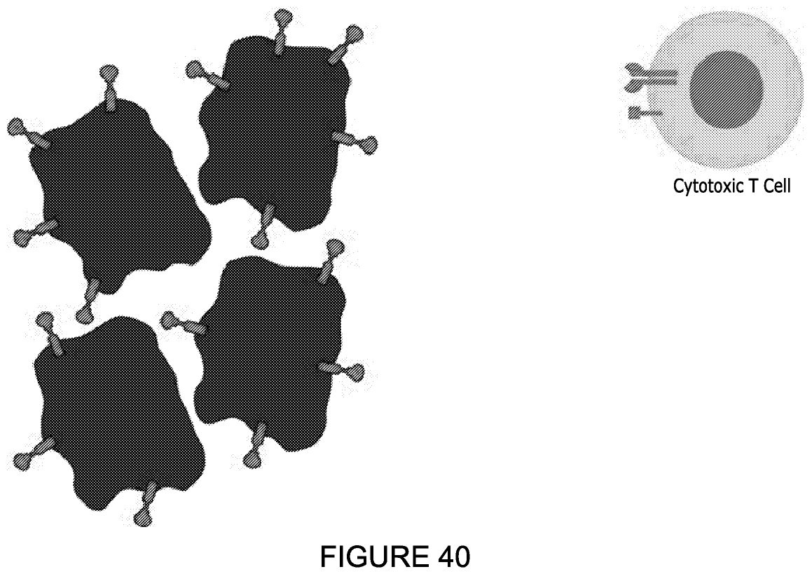

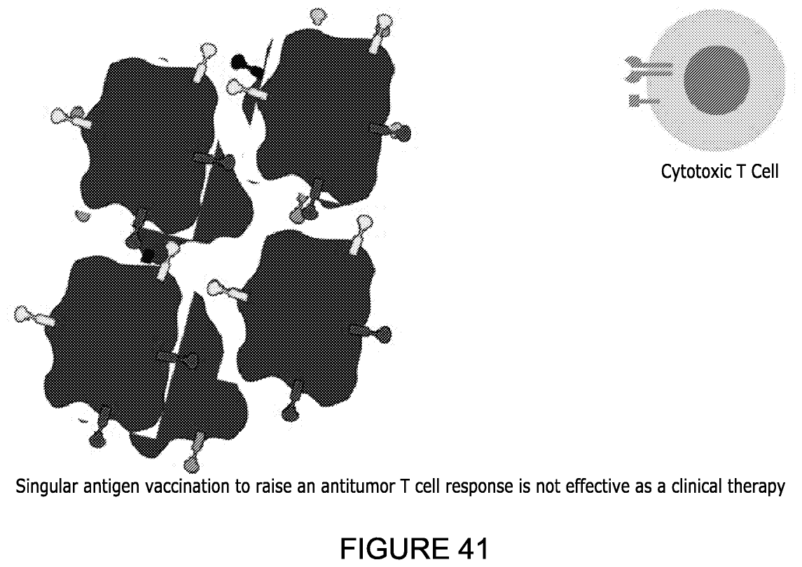

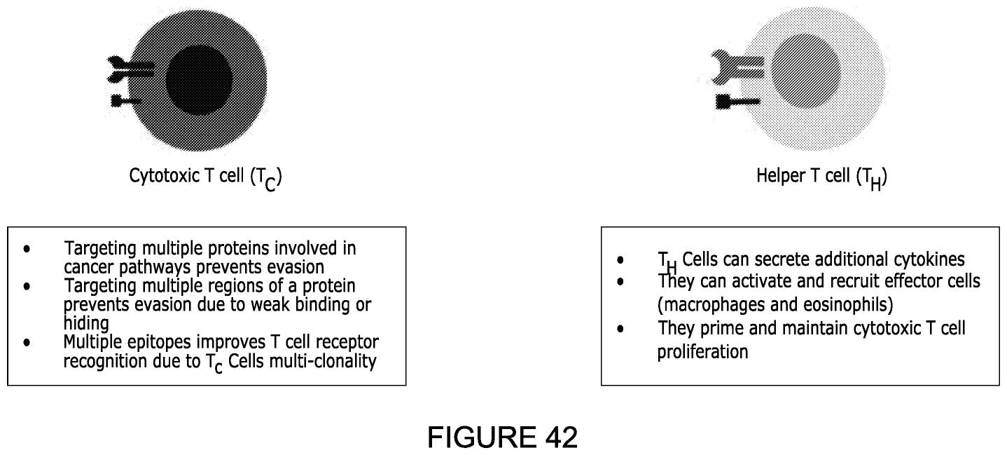

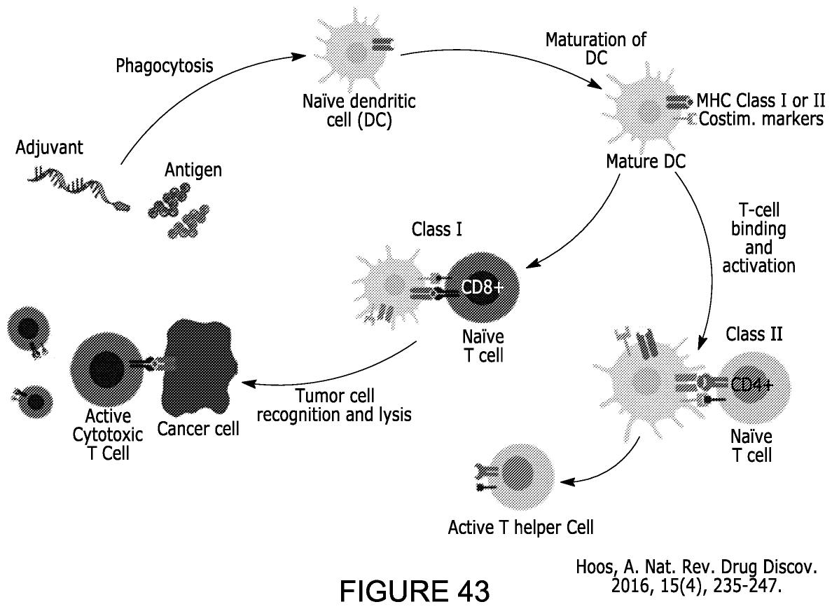

This Example explored the vaccine design space involving multiple cell-targeting antigens. By employing structural changes in antigen placement, the impact of the resulting immune response was elucidated and harnessed to drive success in translational efforts. Antigens used activate cytotoxic (CD8+) T cells to effectively kill tumors, as well as helper (CD4+) T cells to synergize interactions for long-lasting tumor rejection.^{17, 18} CD4+ T cells maintain tumor-directed CD8+ functionality by recruiting them to the tumor site and enhancing their proliferation and effector functions.¹⁹⁻²³ Cancer vaccine pathways with both a cytotoxic and helper T cell antigen can activate different classes of T cells through presentation of the cytotoxic antigen on the MHC-I receptor and presentation of the helper antigen on the MHC-II receptor. (See FIGS. 40-43). See also Hoos, A. Nat. Rev. Drug Discov. 2016, 15 (4), 235-247. Therefore, vaccines described herein consider precise structural placement of both major histocompatibility complex (MHC)-I and -II restricted antigen targets (CD8+- and CD4+-activating, respectively) to prime the immune system most effectively.

Herein, the spherical nucleic acid (SNA) platform was utilized to elucidate the effect of nanoscale structure on multi-antigen immunological processes. The SNA is comprised of a nanoparticle core (e.g., liposome) with a dense, radially arranged surface of oligonucleotides. SNAs are powerful tools to explore these complex relationships because of their biocompatibility,²⁴ ability to rapidly enter cells in high quantities,^{25, 26} potent immune activation when employing toll-like receptor 9 (TLR9) agonist DNA as the shell,²⁷ and modularity that enables the defined nanoscale placement of components using well-known chemistry.²⁸⁻³⁰ In this Example, it is demonstrated how the structures of SNA vaccines carrying multiple immune cell targeting antigens greatly influences immune activation. Changing the position of the antigen type within the SNA upregulated immune cell pathways at the transcriptome level, enhanced production and secretion of cytokines and memory markers at the cellular level, and slowed tumor growth at the organismal level. Collectively, these changes defined vaccine potency against an aggressive B16-F10 melanoma tumor model and, importantly, elucidated design insights regarding multi-antigen placement that translate to other therapeutics.

### Results and Discussion

The experiments described in this Example sought to determine the optimal antigen processing conditions for multi-antigen SNA vaccines to generate robust cytotoxic and helper T cell responses. In particular, how the delivery of two antigen classes (MHC-I and -II restricted) to dendritic cells (DCs) would change processing in vitro was investigated. DCs are critical professional antigen-presenting cells that induce signaling for effective T cell priming. Previous literature has demonstrated the potential to enhance DC activation through simultaneous delivery of both cytotoxic and helper antigens,^{31, 32} but none have had a platform capable of understanding the best way to present such antigens.¹⁴ It was hypothesized that the simultaneous delivery of both antigen classes on the same nanoparticle, as opposed to their delivery on different nanoparticles, enhances the activation of both T cell types, and that the structural location of the antigens markedly impacts vaccine performance.

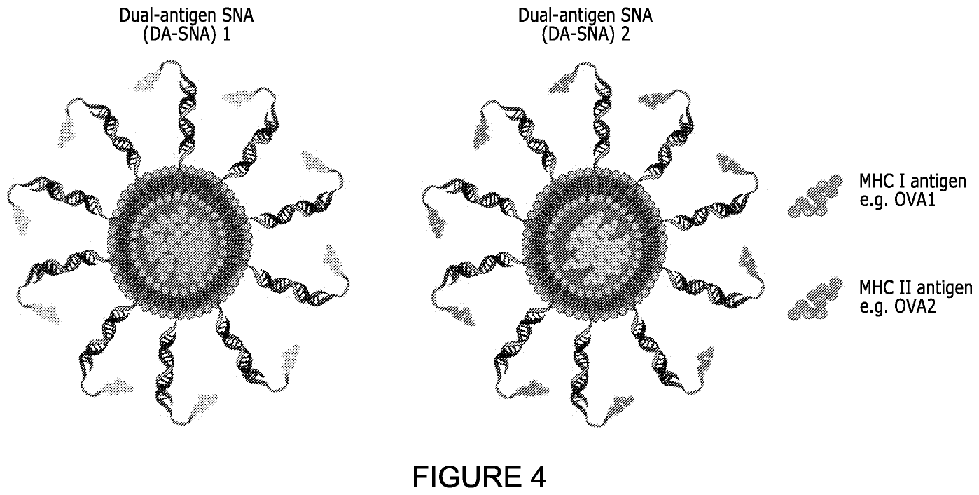

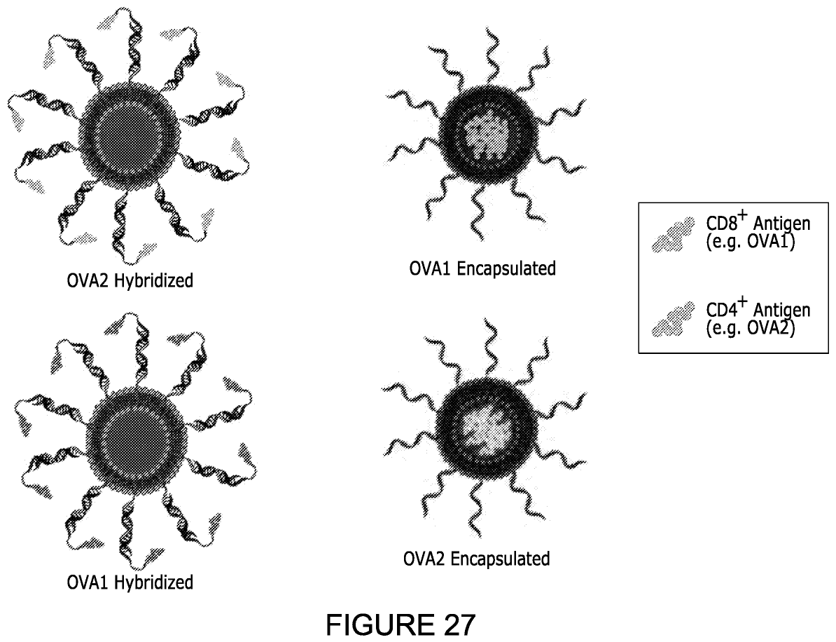

Processing of Multiple Antigens In Vitro Based on Antigen Distribution on SNAs. To test this theory, dual-antigen SNA vaccines (DA-SNAs) that contained both MHC-I and -II restricted antigens in different nanoscale locations (termed DA-SNA 1 and DA-SNA 2 based on placement of each antigen (see, e.g., FIGS. 1, 4, and 26A) were designed and synthesized. Due to the SNA modularity, there are multiple different locations within the SNA construct where antigens can be placed. For this work, encapsulation and hybridization arrangements were selected for antigen placement and compared to one another. To assess how distribution of antigens and delivery on different nanoparticles affected immune activation, formulations containing two individual SNAs, each presenting only one antigen class in the same position as in the DA-SNA vaccine, were synthesized (FIG. 27). The formulations were termed “separate” for the individual SNAs delivering a single antigen and “combined” for the dual-antigen containing DA-SNA.

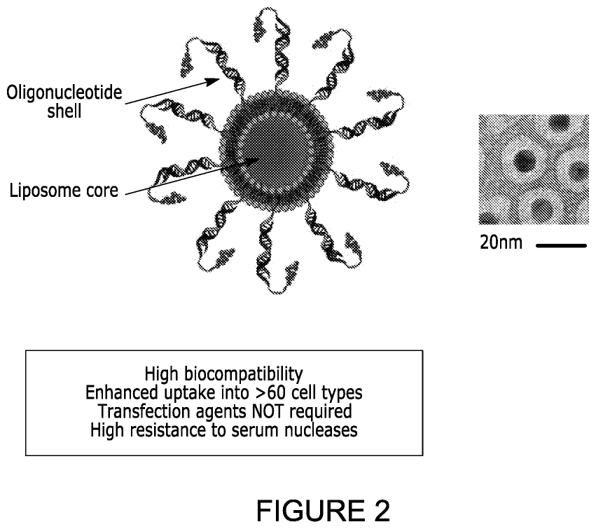

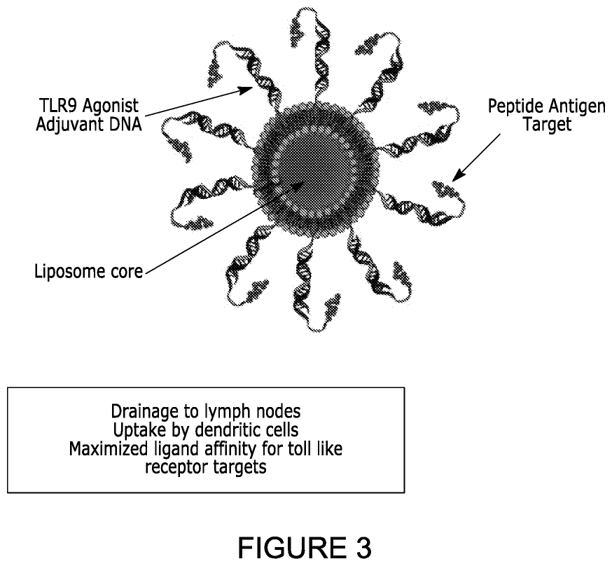

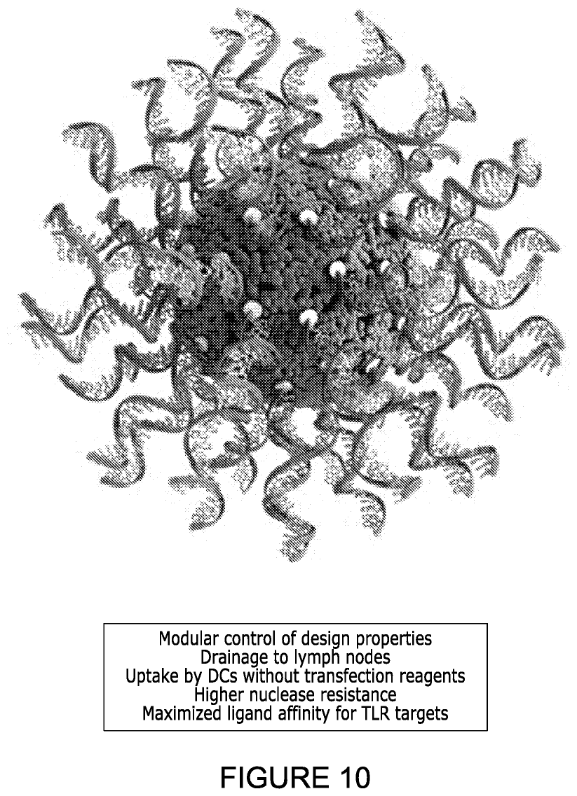

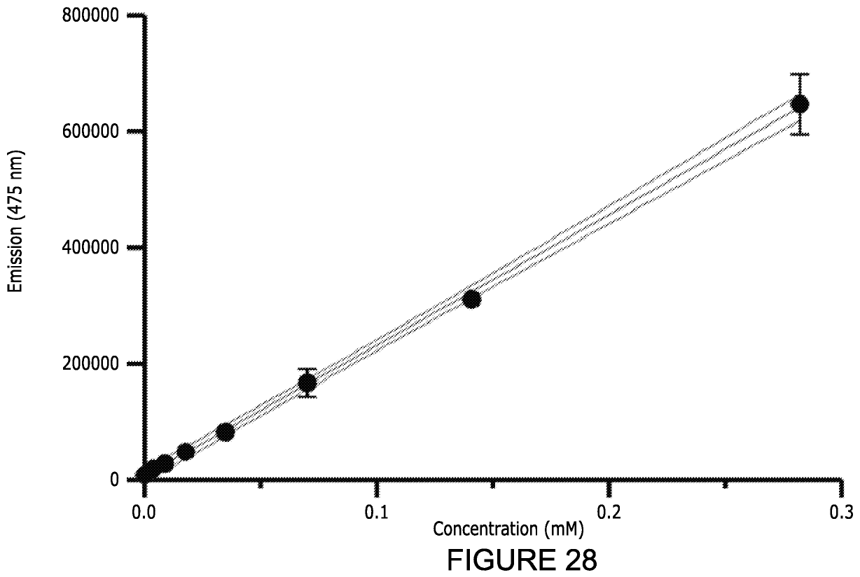

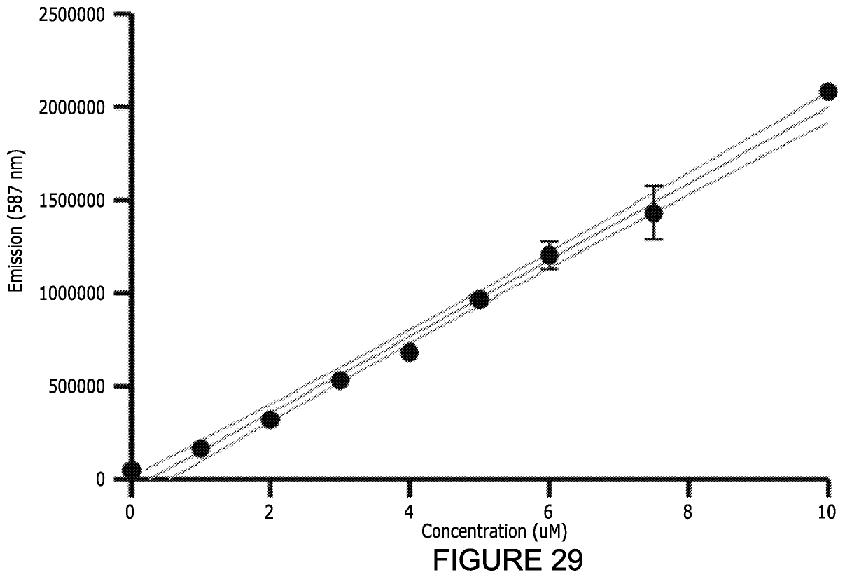

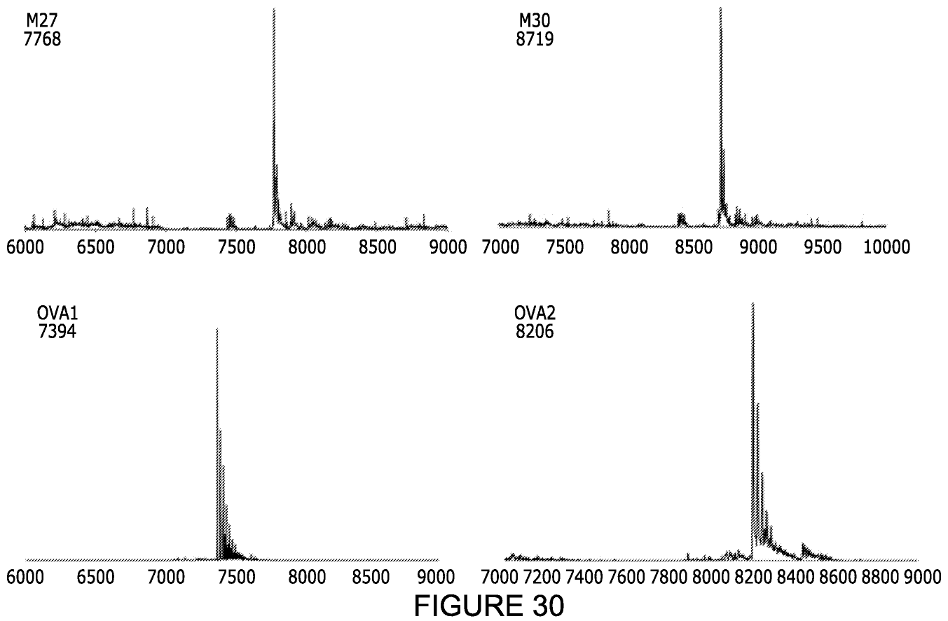

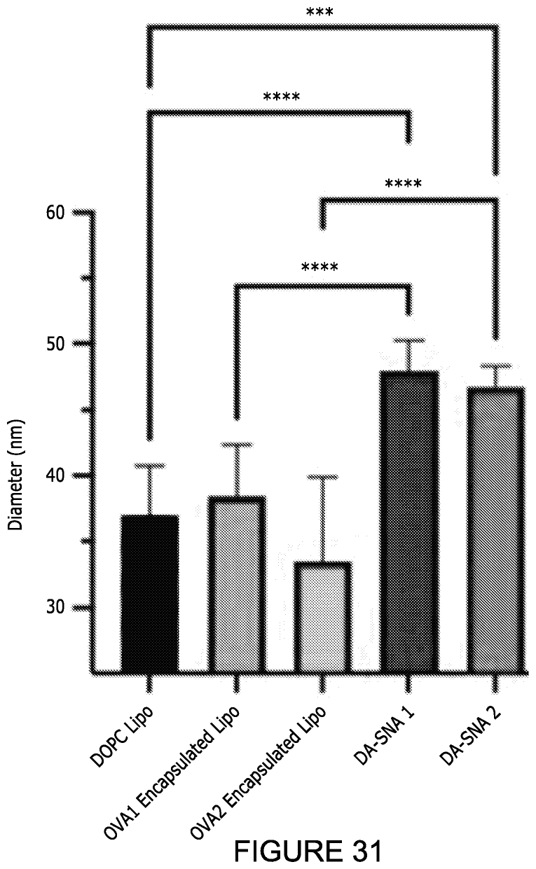

To synthesize DA-SNAs, a peptide from one antigen class was encapsulated into 50 nm 1,2-dioleoyl-sn-glycero-3-phosphocholine (DOPC) liposomes during their formation (FIGS. 28 and 29). In parallel, the peptide of the other antigen class was conjugated to a strand complementary to the CpG motif adjuvant DNA shell (“CpG complement”) of the SNA using disulfide bond formation (FIG. 30). DNA and peptide sequences used in this Example can be found in Table 1 and Table 2). A hybridized duplex was formed by slow-cooling the CpG complement with an appended antigen to a complementary 3′-cholesterol-terminated CpG strand. The cholesterol anchors the duplex to the surface of the liposome. This hybridized product was added to the liposomes to obtain an equimolar amount of each antigen. The liposome surface was backfilled with non-targeting DNA that did not contain antigen to obtain 75 total DNA strands per liposome, equivalent to a density of 1.6 pmol/cm², at which properties associated with SNAs that make them useful in biology are observed (See FIG. 2, FIG. 3, and FIG. 10).¹⁶ SNAs containing either a single encapsulated antigen or a single hybridized antigen (the “separate” formulations) were synthesized following previous protocols.¹⁶ SNA formation was confirmed using dynamic light scattering (FIG. 31).

TABLE 1
Sequences of DNA used in this Example.

| | | | | Ext. | |
|---|---|---|---|---|---|
| | | | Molecular | Coeffecient¹ | SEQ ID |
| Name | Sequence (5′→3′) | Backbone | Weight | L/(mole·cm) | NO: |
| CpG 1826- | TCC ATG ACG TTC CTG ACG | PS | 7809 | 181100 | 1 |
| 3′Chol adjuvant | TT (Sp18)₂ Cholesterol | | | | |
| CpG 1826 | TCC ATG ACG TTC CTG ACG | PS | 7693 | 181100 | 2 |
| adjuvant | TT (Sp18)₂ TT | | | | |
| CpG 1826- | AAC GTC AGG AAC GTC | PO | 6330 | 206700 | 3 |
| 3′SH | ATG GA Thiol | | | | |
| Complement | | | | | |
| CpG 1826- | AAC GTC AGG AAC GTC | PO | 6674 | 206700 | 4 |
| 3′SH | ATG GA Sp18 Thiol | | | | |
| Complement | | | | | |
| with spacer | | | | | |
| CpG 1826- | Thiol Sp18 AAC GTC AGG | PO | 6716 | 206700 | 5 |
| 5′SH | AAC GTC ATG GA | | | | |
| Complement | | | | | |
| with spacer | | | | | |
| T20-3′Chol | TTT TTT TTT TTT TTT TTT TT | PS | 7083 | 162600 | 6 |
| | (Sp18)₂ Cholesterol | | | | |

¹Calculated using IDT's OligoAnalyzer Tool (https://www.idtdna.com/calc/analyzer (Integrated DNA Technologies OligoAnalyzer, 2021))
(PO = phosphate backbone; PS = phosphorthioate backbone; Sp18 = 18-O-Dimethoxytritylhexaethyleneglycol, 1-[(2-cyanoethyl)-(N,N-diisopropyl)]-phosphoramidite (Glen research #10-1918); Cholesterol = 3′-Cholesteryl-TEG CPG (Glen research # 20-2975); Thiol = 3′-Thiol-Modifier C3 S-S CPG (Glen research # 20-2933))

TABLE 2
Sequences of Peptides used in this Example.

| Peptide | | Sequence | Molecular | SEQ ID |
|---|---|---|---|---|
| Name | Protein | (N terminus→C terminus) | weight | NO: |
| OVA₂₅₇₋₂₆₄ | Ovalbumin | SIINFEKL | 963 | 7 |
| (OVA1) | | | | |
| OVA₃₂₃₋₃₃₉ | Ovalbumin | ISQAVHAAHAEINEAGR | 1774 | 8 |
| (OVA2) | | | | |
| M27 | MART-1 | LCPGNKYEM | 1054 | 9 |
| M30 | MART-1 | CSSVDWENVSPELNSTDQ | 2010 | 10 |

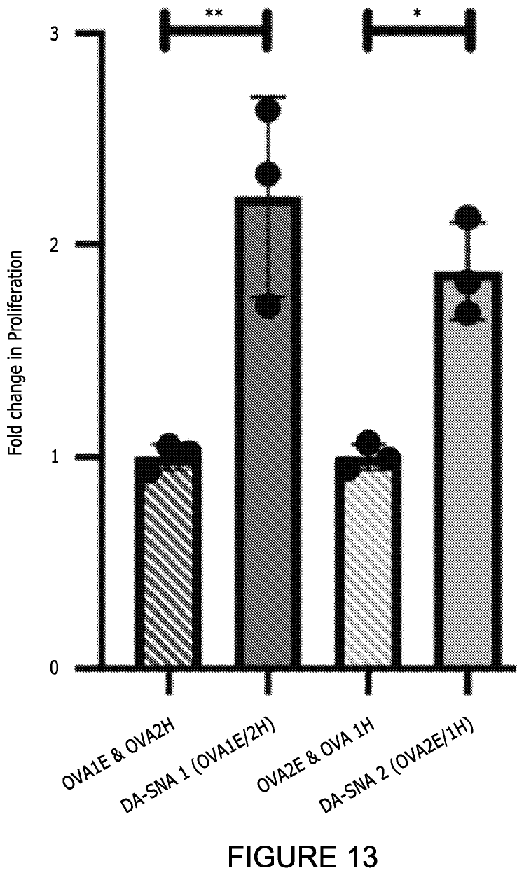

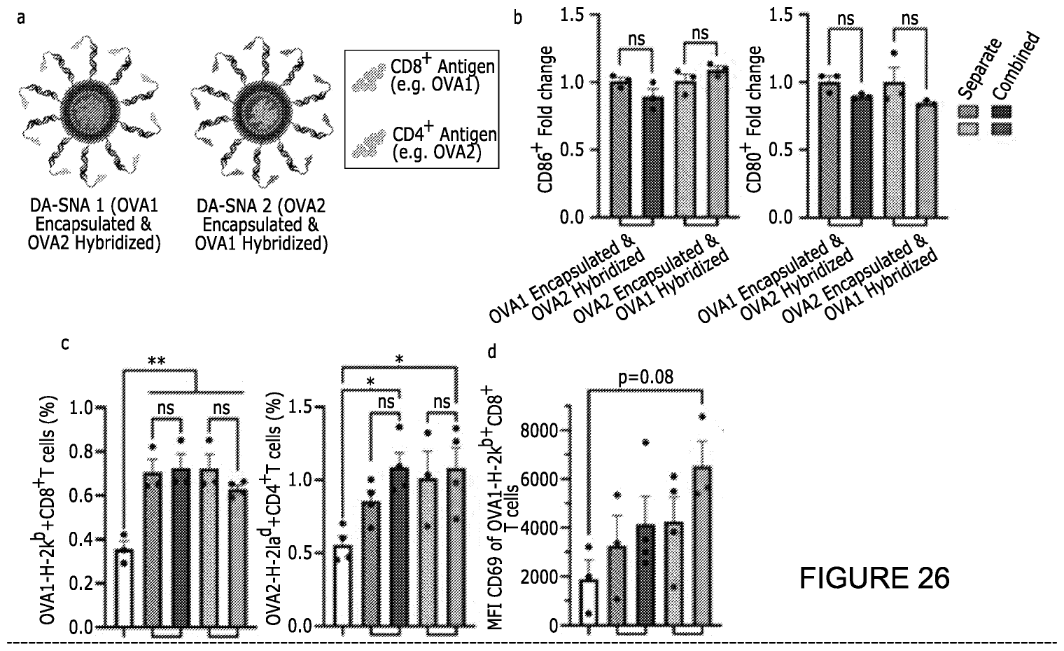

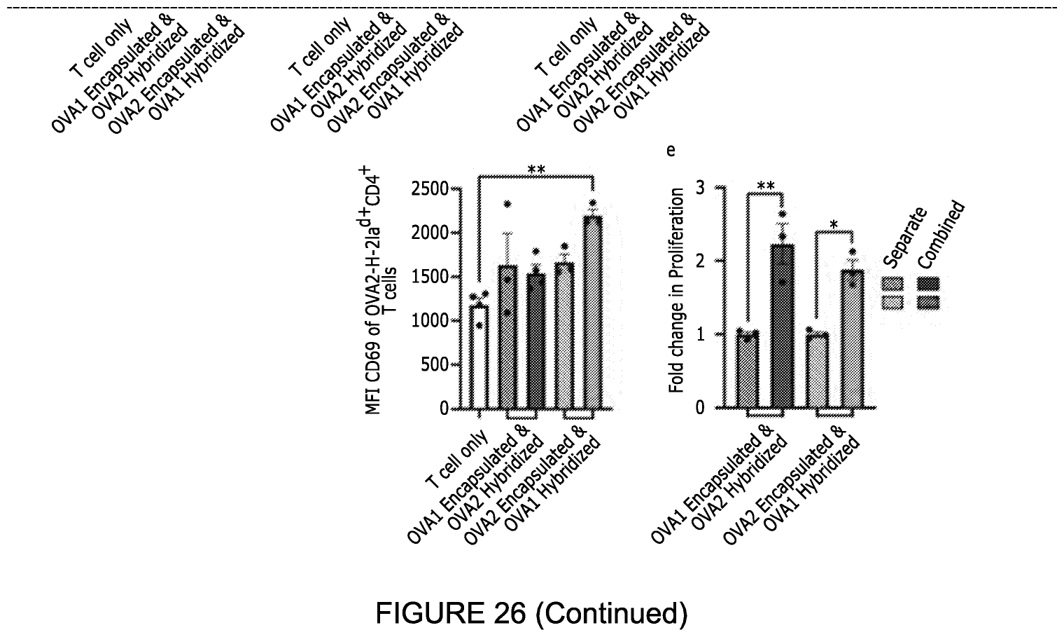

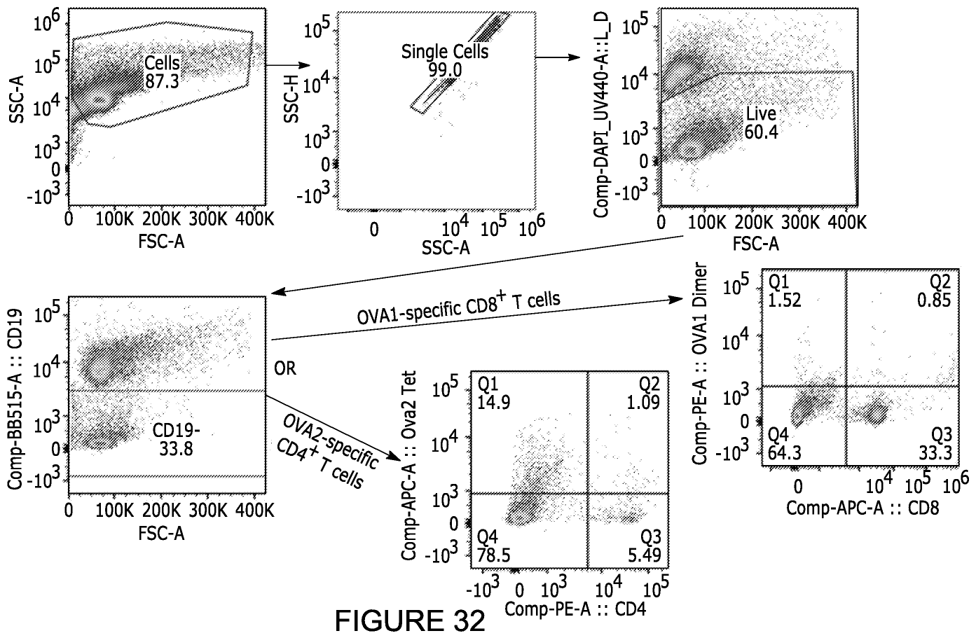

Once these different structures were synthesized, their activation in DCs was assessed using MHC-I and -II restricted model antigens of ovalbumin (OVA), known as OVA1 and OVA2, respectively. The activation of DC cues and the ability to prime T cells was characterized using murine bone marrow-derived DCs and antigens delivered as two separate SNA structures versus the combined DA-SNA vaccine. Specifically, the percentage of CD11c⁺ DCs expressing innate co-stimulation markers CD86 and CD80 did not significantly change depending on the structural arrangement (FIG. 26B). This was likely attributed to the fact that the delivery of adjuvant is equivalent in the combined and separate formulations and indicates that hybridization to CpG does not interfere with TLR9 activation. Moreover, the presentation of antigens to naïve splenic T cells to raise antigen-specific T cells, an indicator of the immune system's ability to recognize tumor antigens,³³ was not impacted by the way antigens were arranged; naïve T cells were able to clonally expand into OVA1- and OVA2-specific T cells (FIG. 26C); gating strategy in FIG. 32. Indeed, ca. 0.6-0.7% of the live CD19− population was double-positive for the OVA1-H-2kb dimer and CD8⁺ marker. Similar trends were observed for OVA2-specific T cell differentiation, with ca. 0.9-1.1% containing double-positive markers for the OVA2-H-2-lad tetramer and CD4⁺ antibody. Only the DA-SNA structures, and not the separate formulations, were capable of significantly elevating the amount of antigen-specific T cells above that of the T cell control baseline; this suggested the combined delivery of both antigens to DCs lead to stronger T cell differentiation. This was more apparent when assessing expression of the early activation marker CD69 within either antigen-specific T cell populations. An increased amount of CD69 signal (as measured by median fluorescence intensity, MFI) was present when the antigens were delivered combined as one DA-SNA, with the DA-SNA 2 structure outperforming all tested groups (FIG. 26D). Moreover, delivery of antigens in DA-SNA structures regardless of the antigen placement translated to a two-fold increase in T cell proliferation, an important step in antitumor responses, using OT1 splenocytes specific for the OVA1 antigen (FIG. 26E, FIG. 13). Based on these findings, DA-SNAs, instead of the separate SNA formulations, were used for all subsequent experiments.

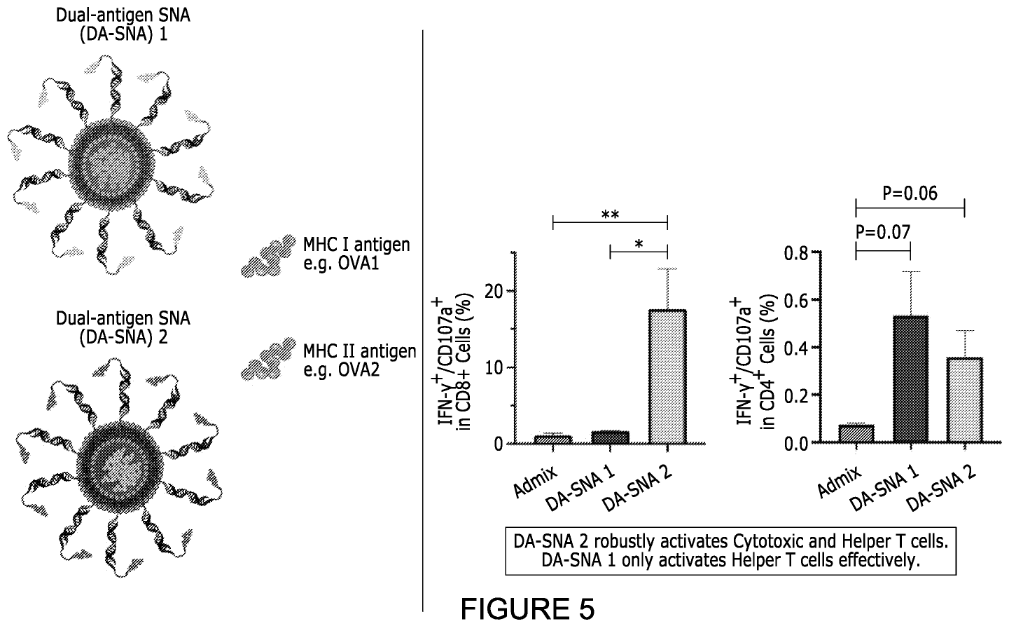

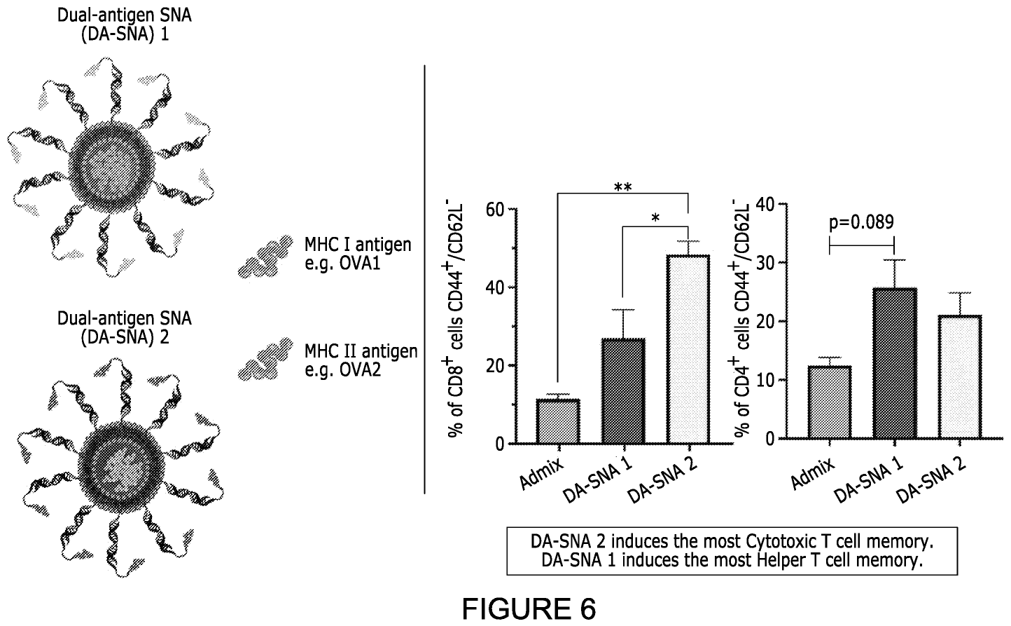

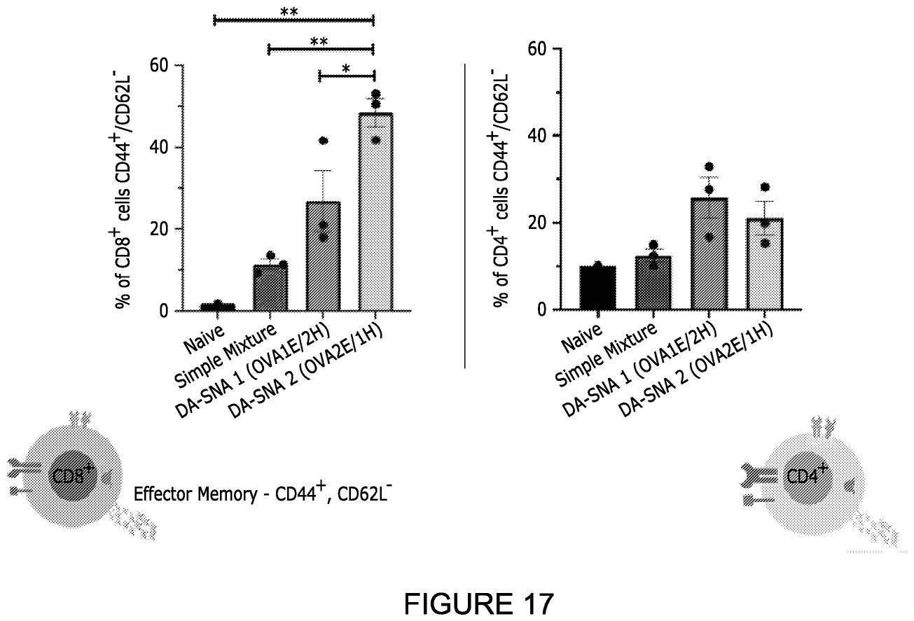

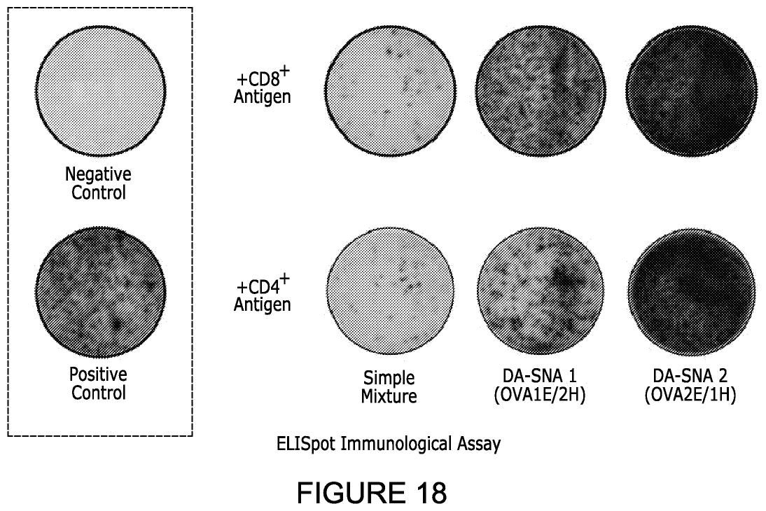

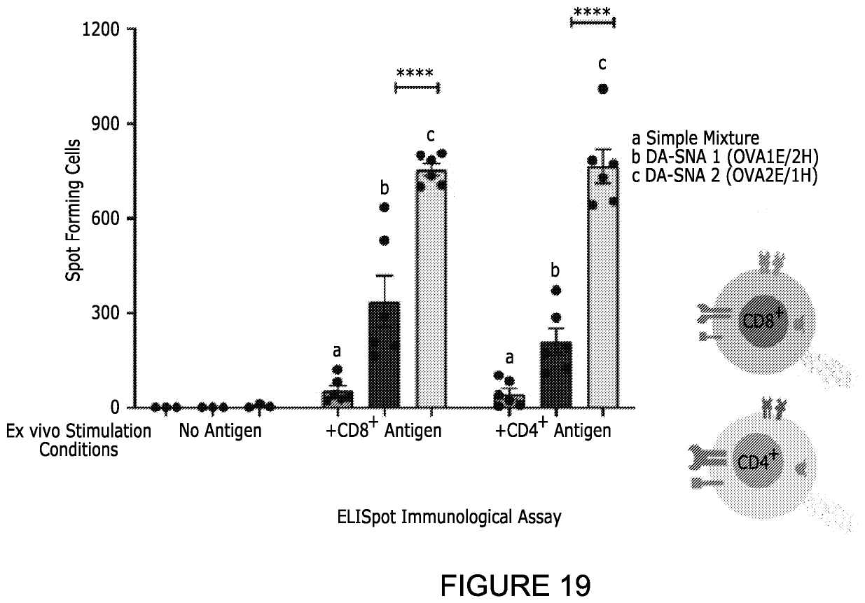

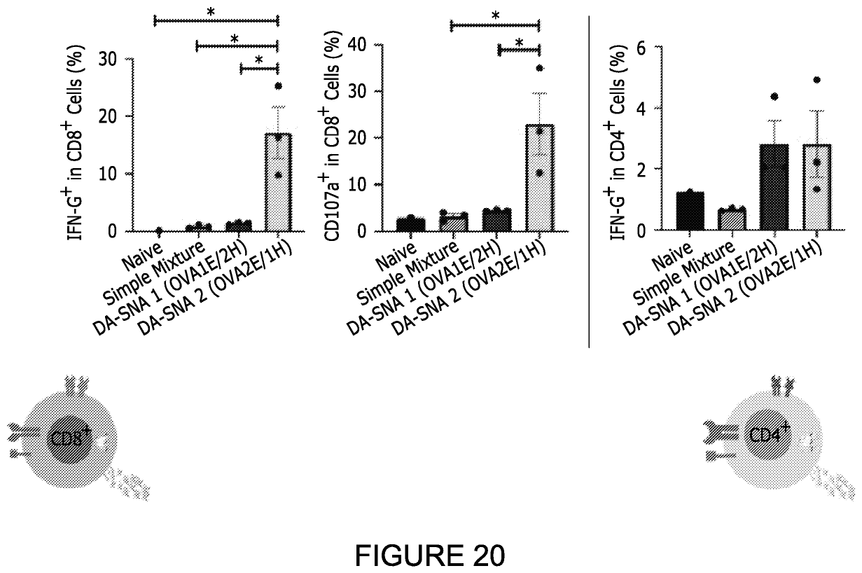

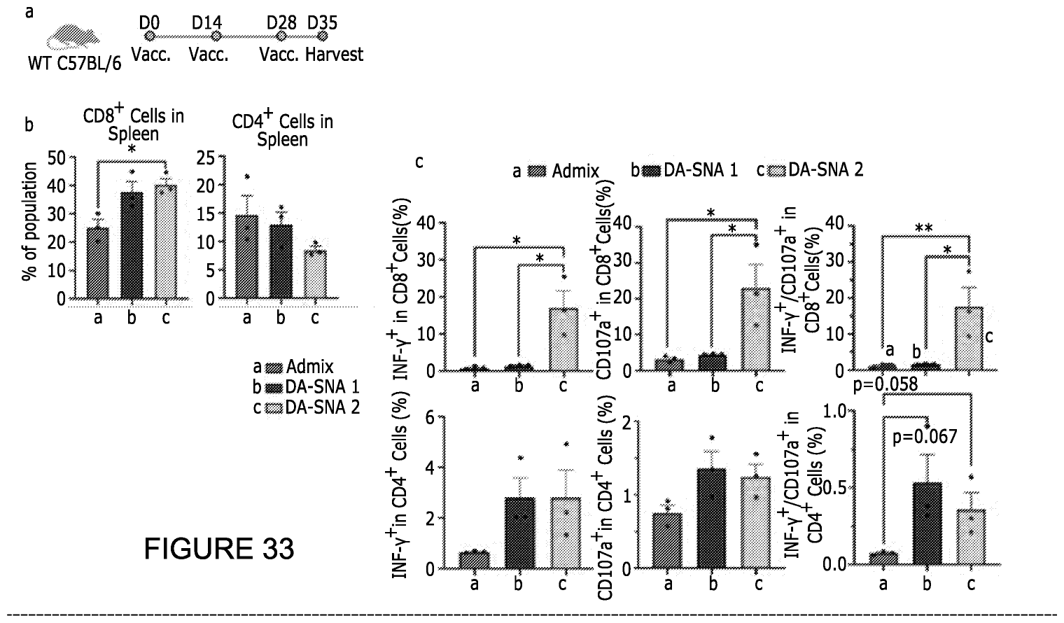

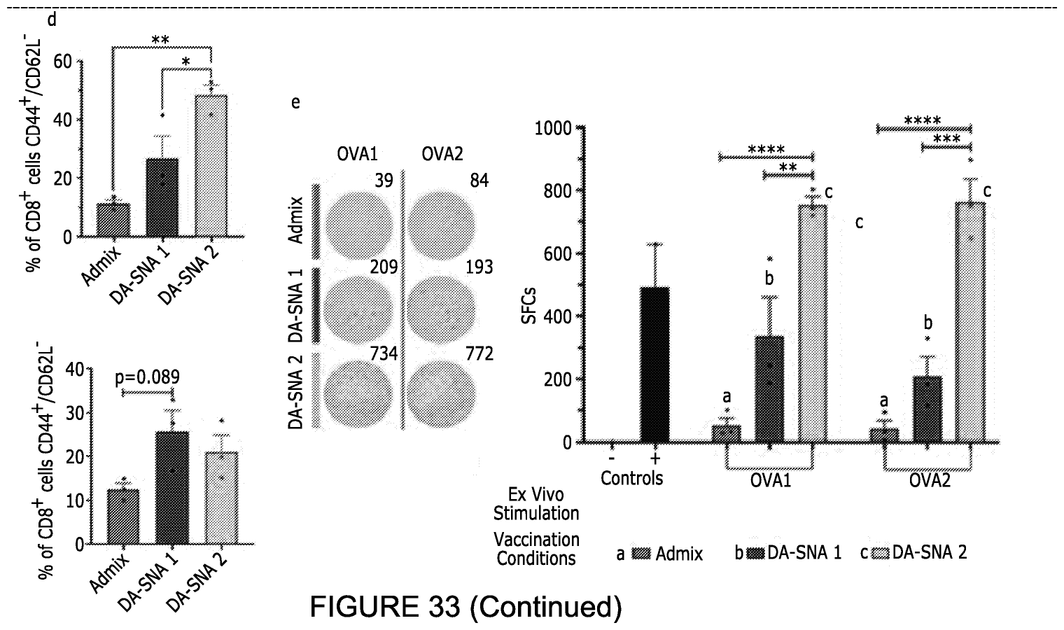

In Vivo Activation via DA-SNAs. Based on these promising in vitro findings using DA-SNAs, C57BL/6 mice were immunized in vivo to delineate how the differences in the placement of the MHC-I and -II restricted antigens within the DA-SNA vaccine affect immune activation. Mice were given three total injections (6 nmol by DNA and each peptide; FIG. 33A). On day 35, splenocytes were harvested to assess raised specific immune responses towards both peptide antigens. After the five week period, CD8⁺ levels were significantly elevated for DA-SNA 2 immunization when compared to a simple mixture containing both peptide antigens and adjuvant DNA was used (termed “admix,” FIG. 33B). CD4⁺ levels were not significantly changed between the treatment groups, although a decrease in helper T cells by ca. 8% as a result of DA-SNA 2 immunization was observed (FIG. 33B). DA-SNA 2 was the only vaccine capable of significantly elevating the production of a key pro-inflammatory cytokine, IFN-γ, as well as degranulation marker, CD107a, upon restimulation with OVA1 peptide ex vivo. DA-SNA 2 immunization also generated a larger percentage of polyfunctional splenic CD8⁺ T cells (ca. 17%, FIG. 33C, FIG. 5, FIG. 20). Moreover, this correlated with an increase in the percentage of effector memory CD8⁺ T cells (CD44⁺CD62L−, ca. 48%) (FIG. 33D, FIG. 6, FIG. 17). Ex vivo stimulation of CD4⁺ T cells with OVA2 peptide showed an overall increase in these same parameters for both DA-SNAs compared to admix treatment, further demonstrating the importance of the combined delivery of both antigens as well as adjuvant to an immune cell; a significant difference was not observed between the two DA-SNA constructs (FIG. 33C-D, FIG. 5, FIG. 6). When evaluating IFN-γ secretion through an enzyme-linked immune absorbent spot (ELISpot) assay, an elevation in spot forming cells (SFCs) was observed for both DA-SNA structures compared to the admix, and the largest enhancement was seen for DA-SNA 2 (FIG. 33E, FIG. 18, and FIG. 19). Furthermore, splenocytes raised by DA-SNA 2 immunization led to at least two-fold more SFCs than DA-SNA 1 immunization when stimulated ex vivo with either OVA1 or OVA2, demonstrating the potency utilizing this arrangement of antigens on a DA-SNA to respond ex vivo to MHC-I or -II restricted antigen cues. Overall, these results highlighted that placement of the MHC-I restricted antigen in the hybridized architecture optimizes presentation for DC processing to potently activate CD8⁺ T cell responses, while encapsulating the MHC-II restricted antigen within the core induced modest enhancements in CD4⁺ activity while preserving cytotoxic function.

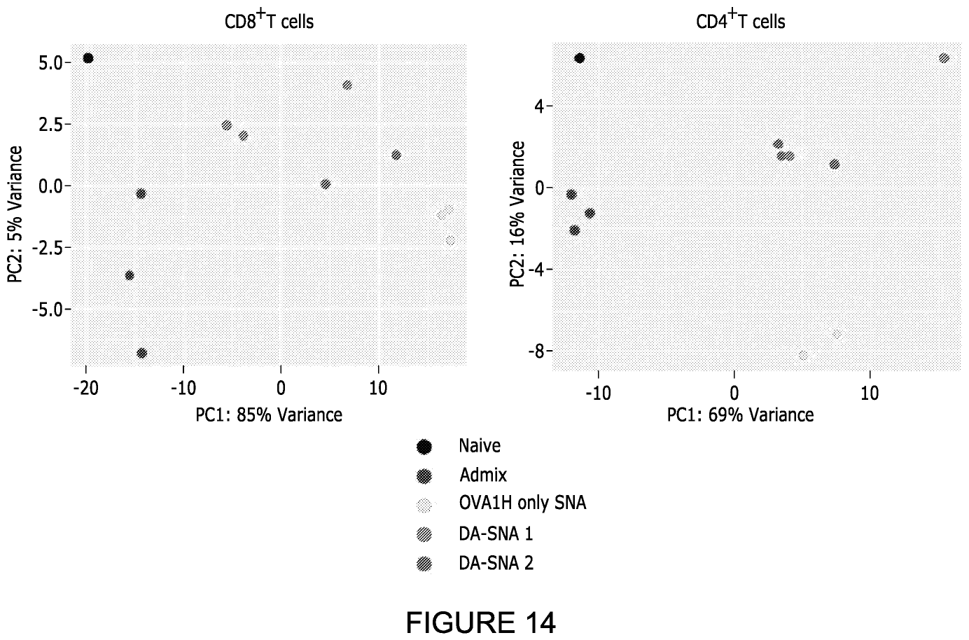

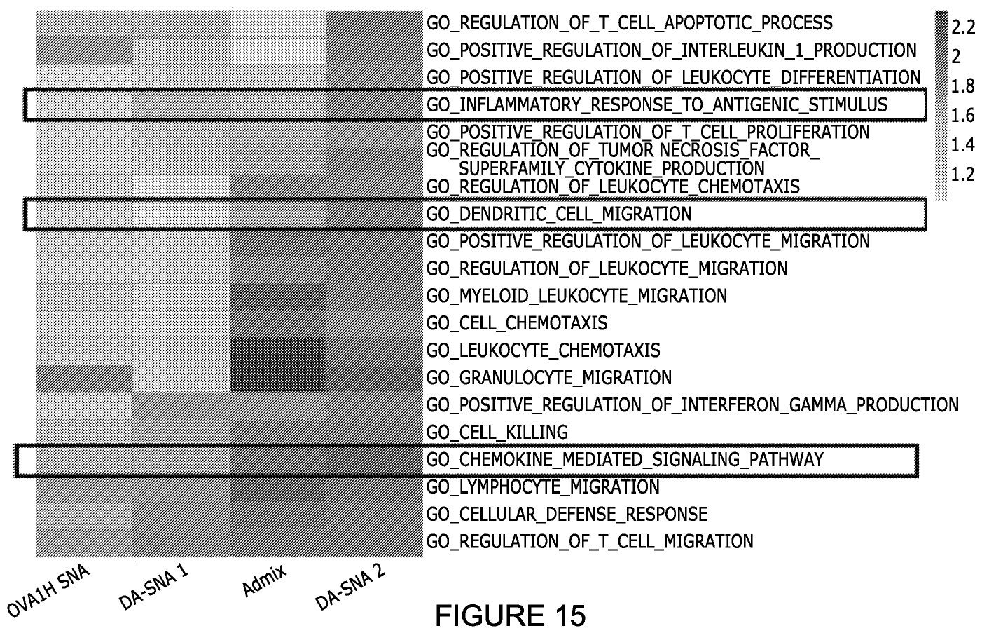

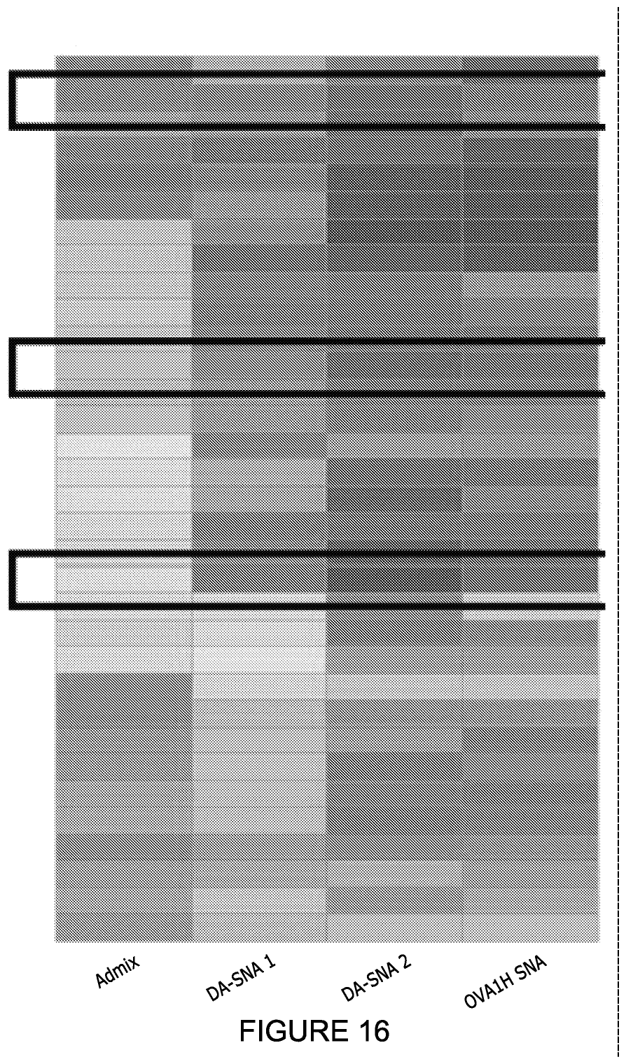

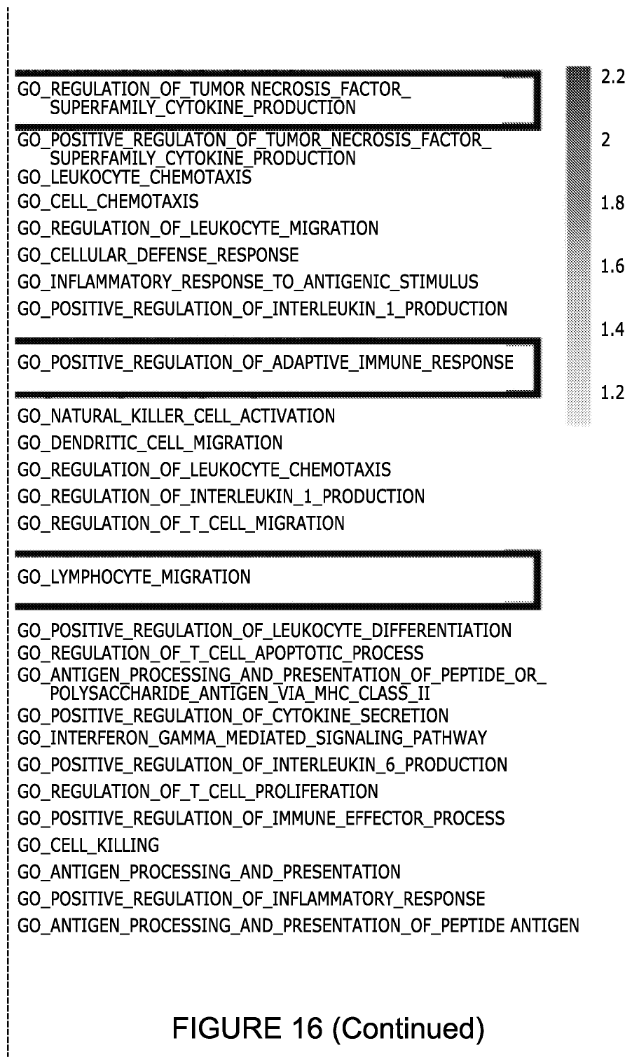

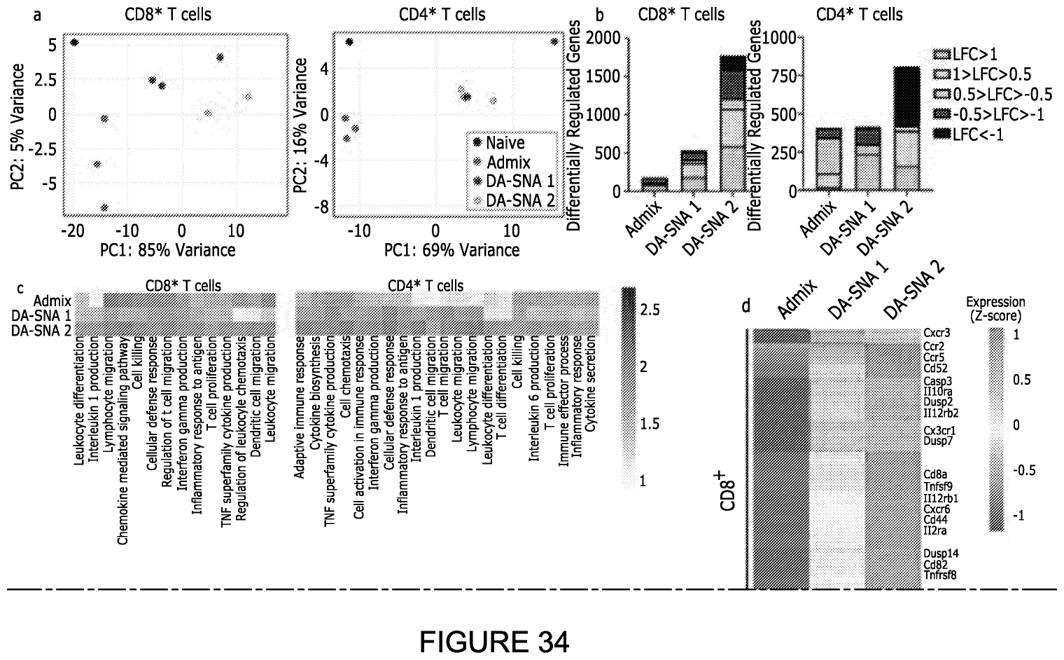

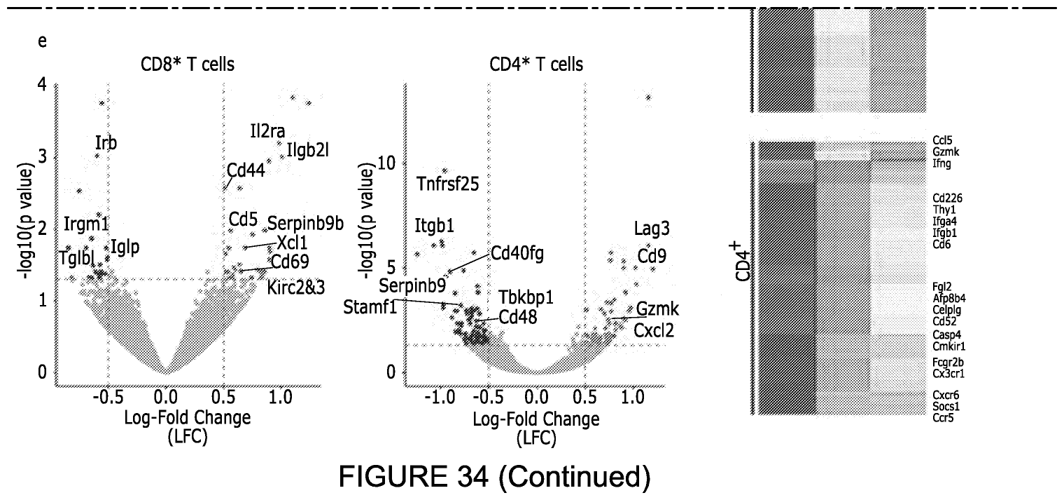

Mechanistic Understanding of DA-SNA induced Immune Activation and Propagation via RNA Sequencing. To understand how the different DA-SNAs and admix treatments were inducing such varied immunological responses in vivo, splenic CD8⁺ and CD4⁺ T cells were collected and isolated after immunization and bulk RNA sequencing (RNAseq) was performed. Principal component analysis (PCA) revealed holistically that the CD8⁺ and CD4⁺ T cell gene expression profiles of mice immunized with admix formulations were most similar to those from naïve mice, suggesting this is the cause of low overall activation (FIG. 34A). Mice immunized with DA-SNA 2 were most distinct from naïve mice in their CD8⁺ transcriptome, whereas both DA-SNA 1 and 2 differed from naïve mice in their gene expression profiles in CD4⁺ T cells in a similar way (FIG. 14). This suggested a rationale for the significant increases in DA-SNA 2-induced CD8⁺ T cell function but similar levels of CD4⁺ T cell function between both DA-SNAs observed in FIG. 33. Moreover, differentially regulated genes for DA-SNA 2 immunized mice exhibited greater absolute log fold changes (LFCs) in both T cell types compared to the other treatments, with at least double the number of differentially regulated genes as a result of DA-SNA 2 immunization compared to DA-SNA 1 (FIG. 34B). Differentially regulated genes were enriched in pathways involving inflammatory responses and upregulation of pro-inflammatory cytokines, chemotaxis, and migration of key immune cell populations (FIG. 34C, FIG. 14, FIG. 15, and FIG. 16). While some of the enriched pathways from DA-SNA 2 treatment were shared with admix treatment and others with DA-SNA 1 treatment, overall, the widespread activation induced at the transcriptome level for the DA-SNA 2 architecture correlated with enhanced immunological outputs.

Relevant gene signatures were identified for adaptive and innate immune activation and functioning across all treatments and include, for example, CXCR3, TNFSF9, and GZMK (FIG. 34D). These genes have particular relevance in T cell effector function and trafficking, antigen presentation and generation of cytotoxic T cells, and helper T cell cytolytic function, respectively. A particular comparison of DA-SNA 2 versus DA-SNA 1 demonstrates unique nanoscale-induced genetic differences induced simply by altering the placement of antigen class (FIG. 34E). A total of 452 and 229 overlapping significant genes in CD8⁺ and CD4⁺ T cells, respectively, were detected between both DA-SNAs. Specifically, DA-SNA 2 induced higher expression of IL2RA, CD44, XCL1 in CD8⁺ T cells and LAG3, CCR7, CCL9 in CD4⁺ T cells compared to DA-SNA 1. Ultimately, comparing gene signatures across all immunization treatments highlighted the substantial impact that vaccine structure and in particular, nanoscale antigen placement, had on genome and expression patterns. These results underscored the immunological measurements that were detected, providing mechanistic rationale that highlighted pathways leading to T cell activation and durable responses, and detail a framework for vaccine design using purposeful structure considerations.

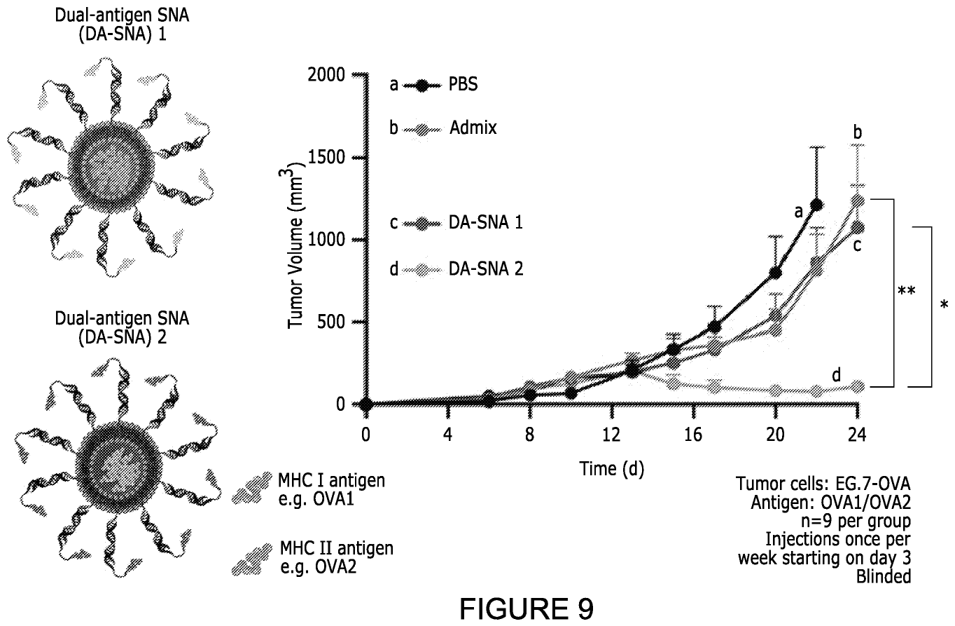

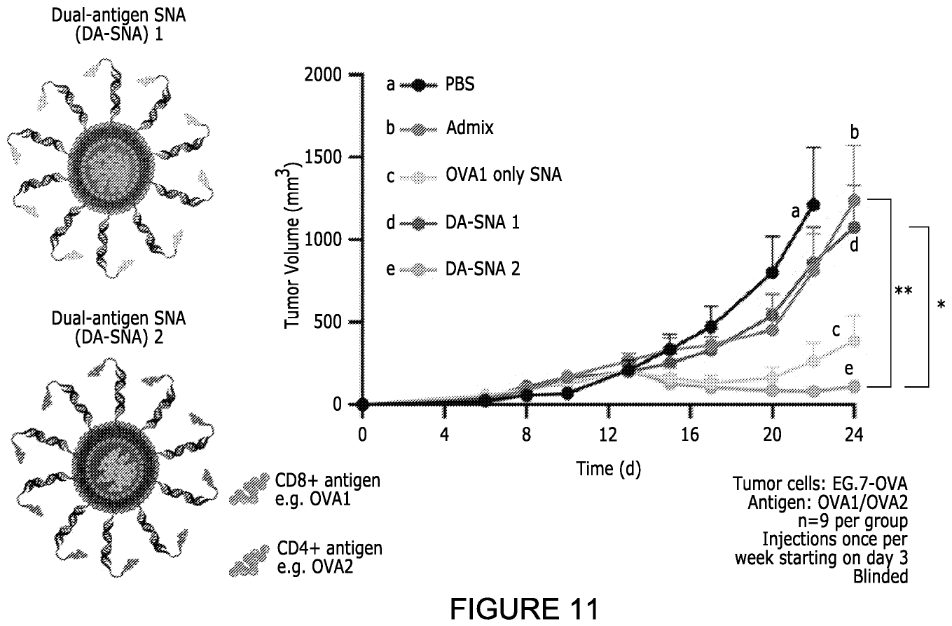

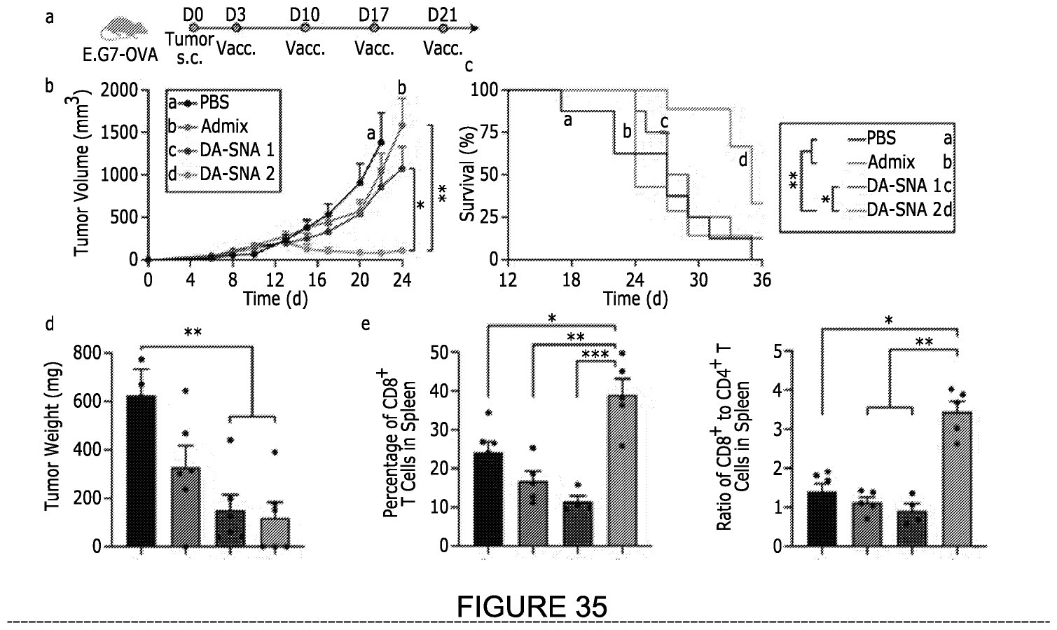

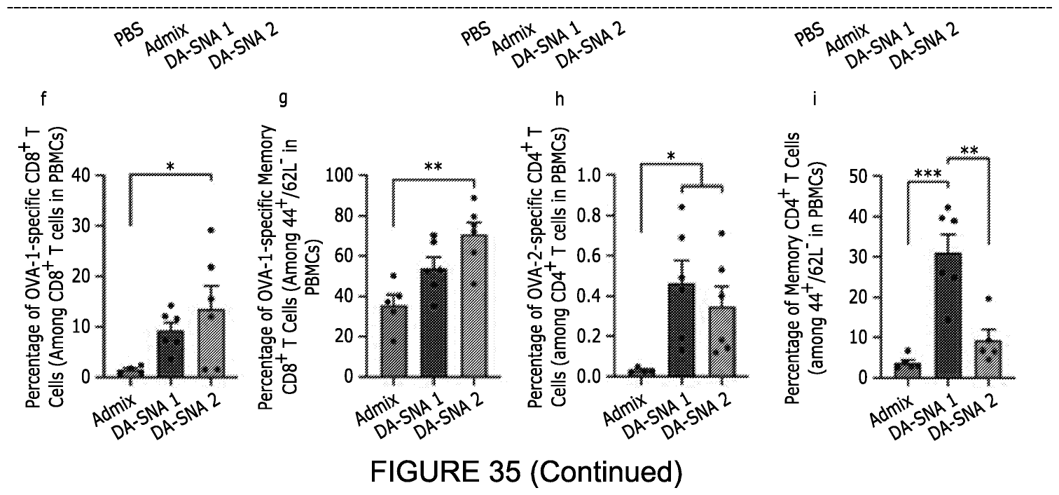

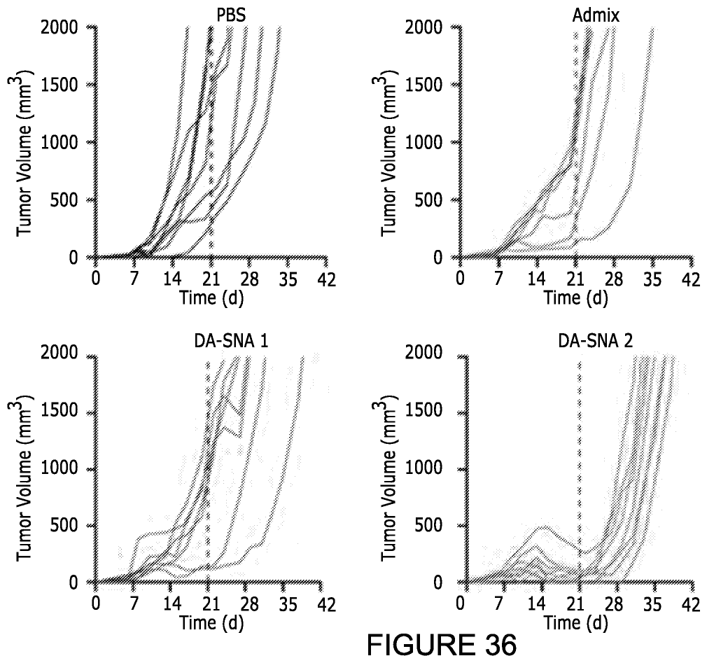

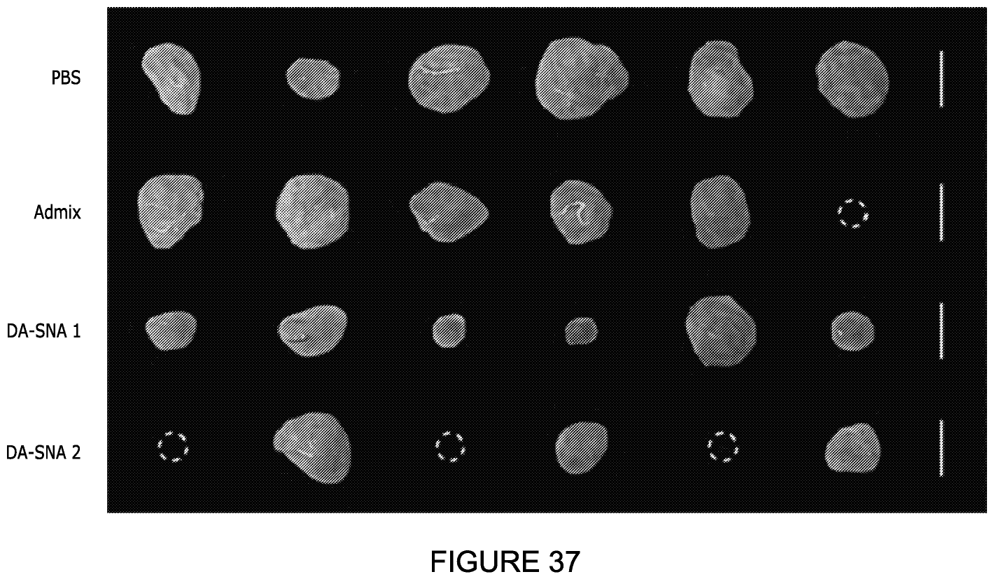

DA-SNA Structure-driven Tumor Inhibition and Immune Activation. To evaluate the therapeutic efficacy and immunological impact of DA-SNAs, we employed a murine E.G7-OVA lymphoma cancer model due to its stable expression of the OVA protein, expressing both the OVA1 and OVA2 epitopes used above.³⁴ Briefly, C57BL/6 mice were inoculated subcutaneously with E.G7-OVA cells and immunized weekly with either DA-SNA or admix formulations (6 nmol of each OVA1 and OVA2 antigen, 6 nmol of adjuvant DNA) (FIG. 35A). Tumor-bearing mice immunized with DA-SNA 2 demonstrated a ca. 3-fold reduction in tumor growth compared to both control (saline-treated) and admix groups as soon as five days after the second immunization (day 15) and more than a 16-fold difference in tumor growth when compared to saline-treated mice 22 days post-tumor inoculation (FIG. 35B, FIG. 9, and FIG. 36). Importantly, DA-SNA 1 treatment did not effectively halt tumor growth compared to admix, unlike DA-SNA 2 treatment. Compared to the admix and DA-SNA 1, DA-SNA 2 produced a ca. 7-fold reduction in tumor growth at day 24, highlighting the pronounced impact of antigen positioning and, ultimately, translating to a significant extension in animal survival (median survival in days: PBS=27; Admix=24; DA-SNA 1=28; DA-SNA 2=35) (FIG. 35C, FIG. 11). To further investigate the physical impact of treatment on tumor growth, tumors were excised from mice at day 15 following the same treatment regimen and subsequently weighed (FIG. 35D and FIG. 37). Interestingly, at this point in the tumor growth curve, both SNA groups showcased a significant reduction in tumor weights when compared to PBS-treated mice, suggesting that DA-SNA 1 was capable of raising an antitumor immune response, but that it was not as durable as that raised from DA-SNA 2.

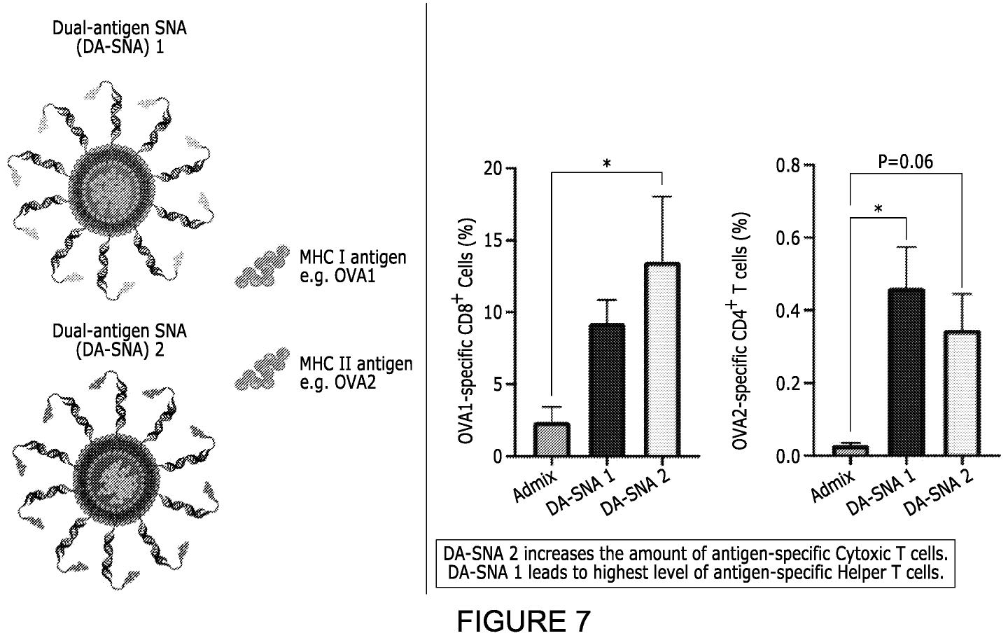

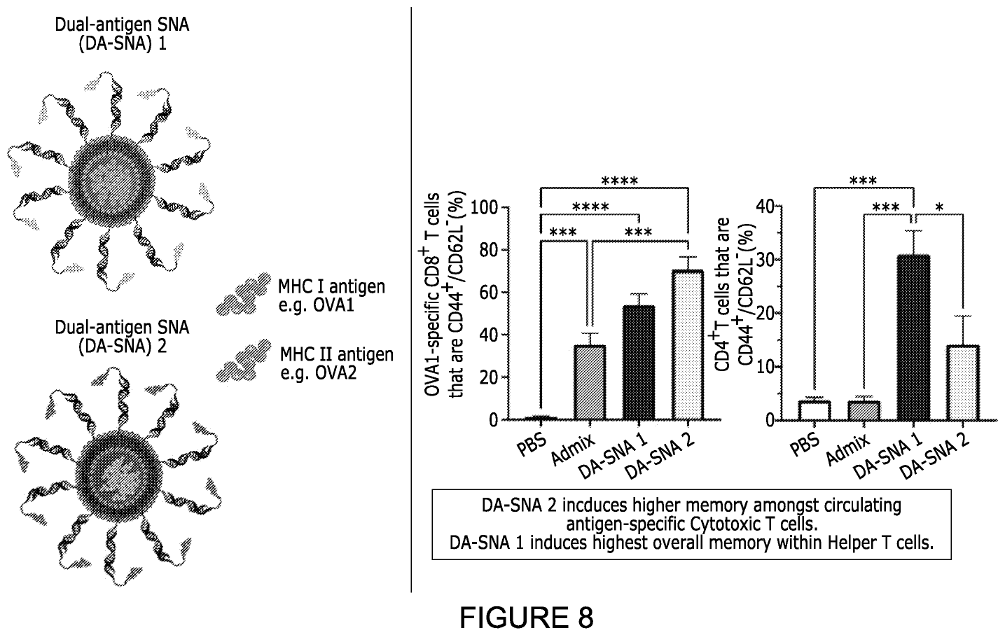

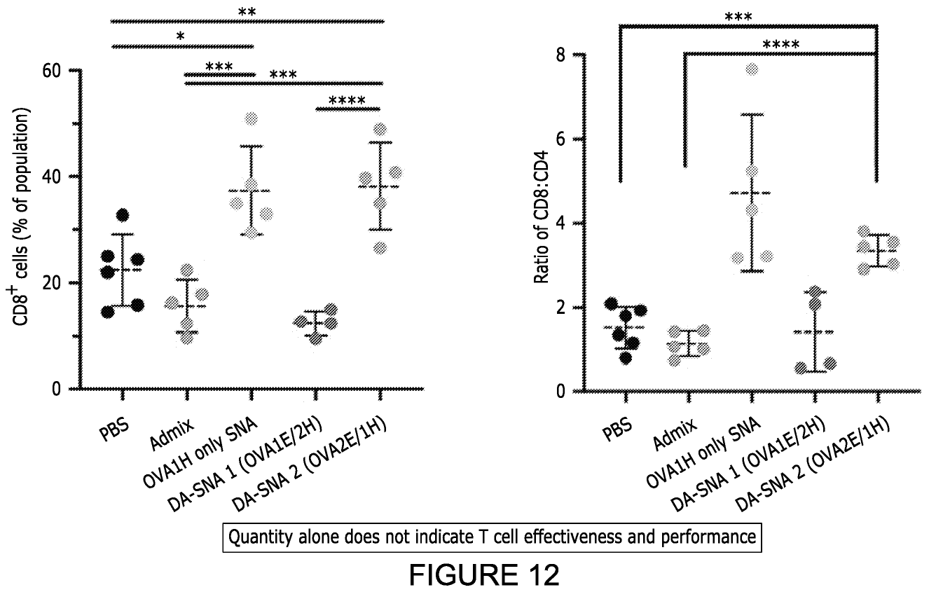

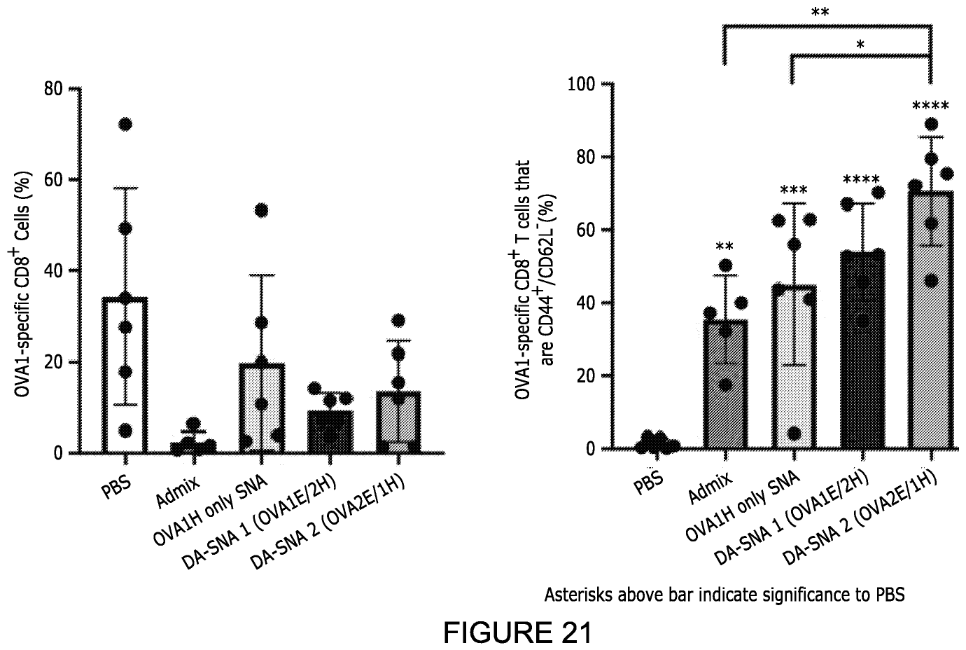

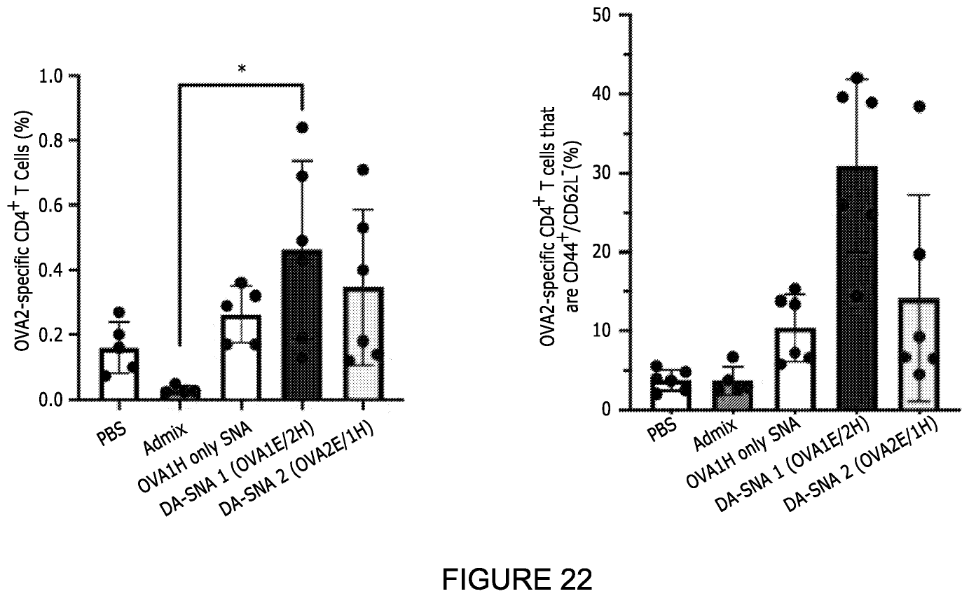

Spleens were harvested and evaluated for changes in CD8⁺ and CD4⁺ T cells to elucidate differences as a result of treatment that may have contributed to tumor reduction (FIG. 35E, FIG. 12). The spleens of DA-SNA 2-treated mice generated a significantly higher percentage of CD8⁺ T cells when compared to other treatment groups and also displayed an overall higher ratio of CD8⁺ to CD4⁺ T cells. To evaluate the immunological differences that contributed to tumor suppression, tumor-bearing C57BL/6 mice were assessed for circulating peripheral blood mononuclear cells (PBMCs) on day 15, when differences in tumor growth were first observed and when the impact of DA-SNA 2 treatment began to halt tumor growth while the other treatments had negligible impact. Notably, DA-SNA 2-treated mice showcased the highest level of circulating antigen-specific CD8⁺ T cells (FIG. 35F, FIG. 7, FIG. 21). This subset of CD8⁺ lymphocytes was further evaluated for their memory phenotype. In this case, DA-SNA 2 treatment significantly elevated the effector memory phenotype to over 60% of OVA1-specific circulating CD8⁺ T cells (FIG. 35G, FIG. 8, FIG. 22). Antigen-specific CD4⁺ T cells were also significantly elevated for mice treated with the DA-SNAs (FIG. 35H, FIG. 7, FIG. 21). As expected, due to the transcriptome profiles and immunological parameters previously explored herein for CD4⁺ T cells, there were negligible differences between the two DA-SNA groups. While there were not enough OVA2-specific CD4⁺ T cells to accurately delineate the memory phenotype within this subpopulation, the entirety of CD4⁺ T cells demonstrated an enhanced effector memory state when treated with DA-SNA 1 (ca. 30% of CD4⁺ T cells), compared to treatment with DA-SNA 2, which matured ca. 10% of CD4⁺ T cells (FIG. 35I, FIG. 8).

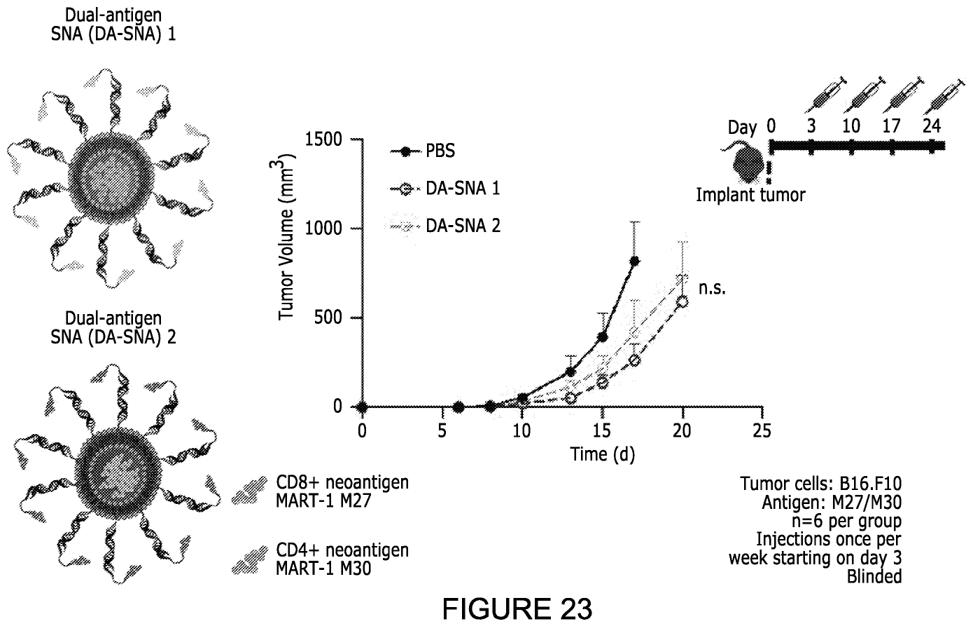

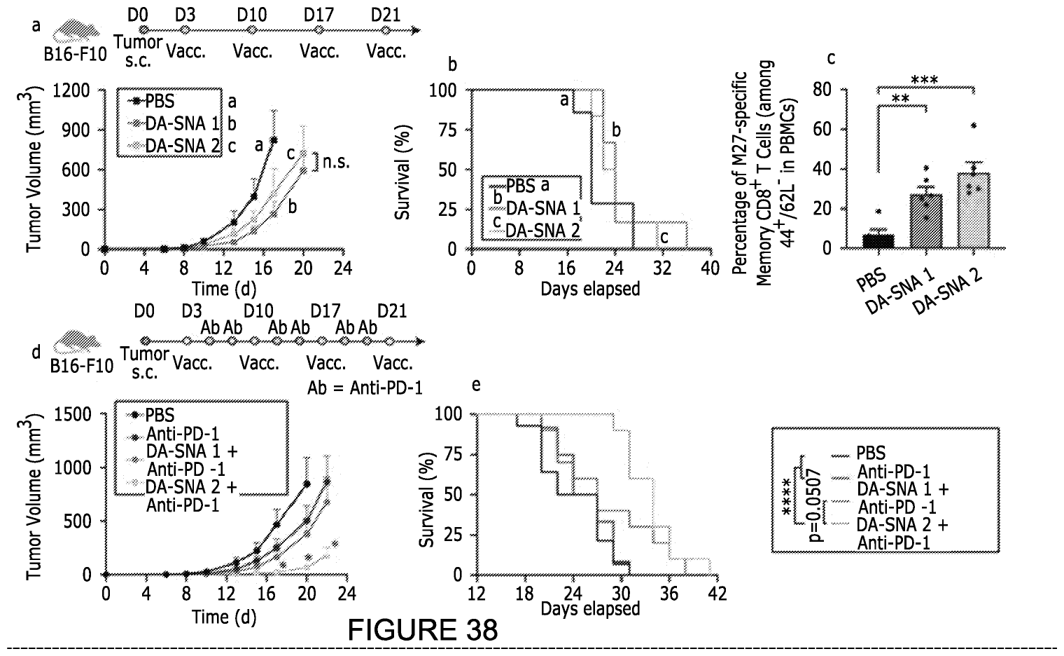

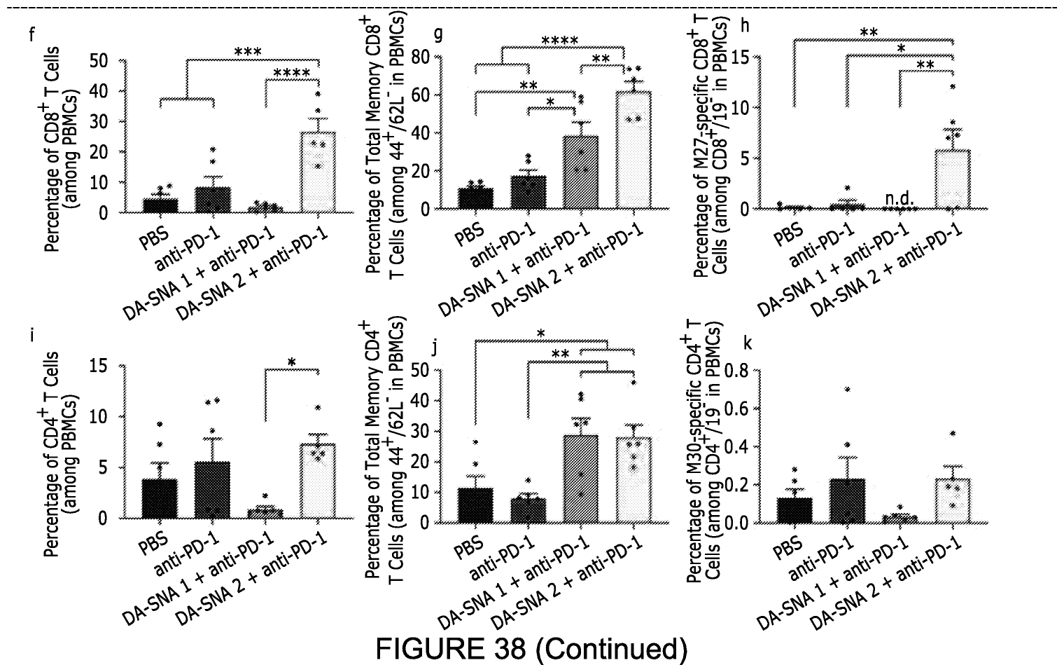

Structural Impact of Antigen Placement in Clinically-relevant Melanoma Tumor Model. The findings learned regarding DA-SNA structure heretofore in model systems were utilized to assess the ability of dual-antigen placement to impact growth of B16-F10 melanoma tumors. This model has been established as highly aggressive with enhanced immunosuppressive properties.³⁵ The recently reported³⁶ M27 and M30 neo-epitopes, from the MART-1 protein containing mutations present only in the tumor, were selected as MHC-I and -II antigens, respectively. Initially, C57BL/6 mice inoculated with B16-F10 tumor cells receiving a weekly vaccination of either DA-SNA 1 or DA-SNA 2 showcased a substantial percent inhibition in tumor growth at day 17 compared to saline-treated mice (68% and 48%, respectively) (FIG. 38A-B, FIG. 23). Indeed, a statistically significant ca. 4-fold increase in circulating effector memory antigen-specific CD8⁺ T cells was observed for mice treated with either DA-SNA relative to saline treatment (FIG. 38C). Therefore, an antigen-specific immune response was produced, but the negligible impact this had on tumor growth indicates the likelihood of a highly immunosuppressive tumor environment that was inhibiting the therapeutic potential of these T cells.

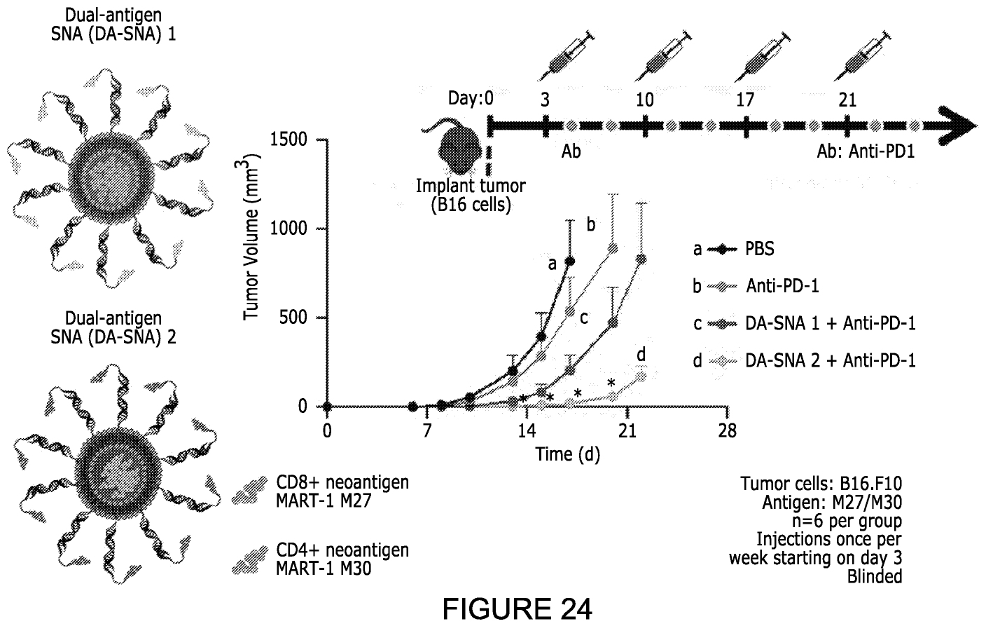

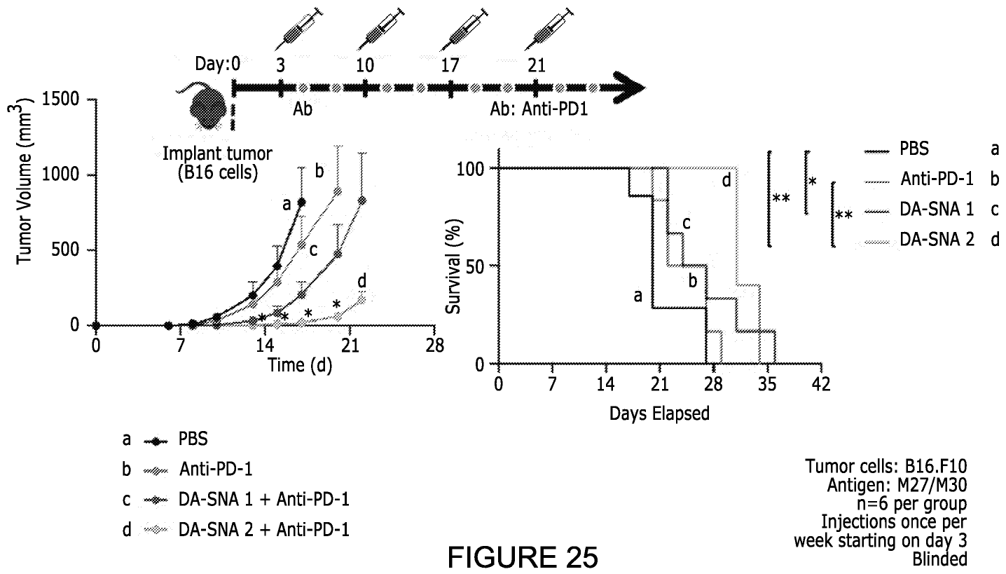

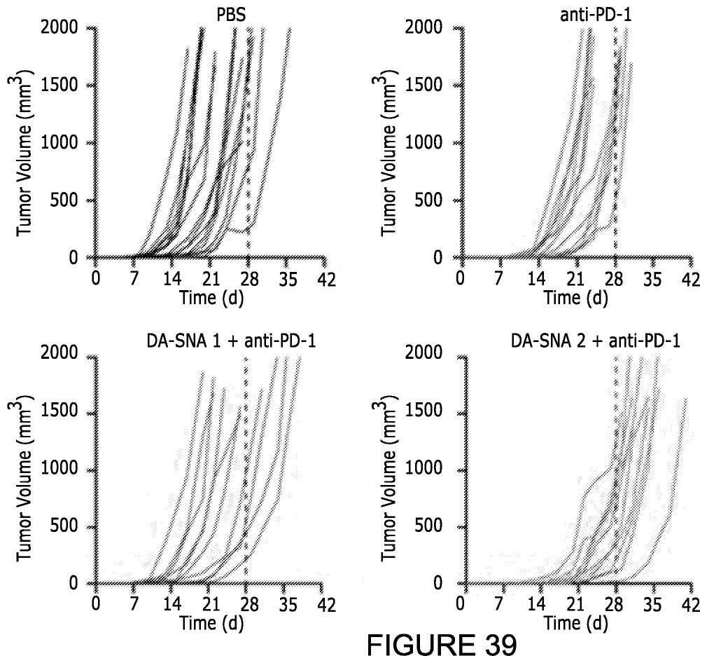

Due to these observations and the inherent aggressiveness of this tumor model, DA-SNA treatments were combined with the immune checkpoint inhibitor anti-PD-1, an FDA-approved treatment for advanced melanoma, in an effort to overcome the tumor's immunosuppression.^{37, 38} Notably, when anti-PD-1 was co-administered with DA-SNAs, a significant decrease in tumor growth was observed beginning as early as 17 days post-tumor inoculation for animals treated with the combination DA-SNA 2+anti-PD-1 therapy, while no substantial decrease in tumor growth was observed for mice in the other treatment groups (FIG. 38D). See also FIG. 24, FIG. 25, and FIG. 39. This translated to a 40% extension in median survival for these mice compared to those in the saline-treated or anti-PD-1 monotherapy-treated groups (FIG. 38E). Moreover, a comparison between DA-SNA combination treatment groups revealed a substantial improvement in overall median survival for combination DA-SNA 2+anti-PD-1 animals (p=0.0507). The evaluation of immune cells isolated from peripheral blood further highlights this dramatic structure-induced difference where DA-SNA 2 appears to work synergistically with the checkpoint inhibitor. A significant increase in circulating CD8⁺ T cells for animals receiving a combination DA-SNA 2+anti-PD-1 treatment was observed compared to all other groups (FIG. 38F). Interestingly, when the total CD8⁺ effector memory T cell population was evaluated amongst these circulating PBMCs, significant increases were detected for both combination DA-SNA+anti-PD-1 groups, with DA-SNA 2+anti-PD-1 generating effector memory phenotypes in ca. 60% of circulating CD8⁺ T cells (FIG. 38G). Moreover, only this combination DA-SNA 2+anti-PD-1 treatment was able to induce a robust antigen-specific CD8⁺ T cell response (FIG. 38H). Observations of CD4⁺ T cell circulation revealed a similarly high increase as a result of combination DA-SNA 2+anti-PD-1 therapy (FIG. 38I), although both combination DA-SNA+anti-PD-1 groups significantly elevated the effector memory phenotype to ca. 28% of the CD4⁺ T cell population (FIG. 38J). The combination DA-SNA 2+anti-PD-1 treatment increased levels of antigen-specific CD4⁺ T cell production along with anti-PD-1 monotherapy compared to DA-SNA 1+anti-PD-1 treatment, although significant differences were not observed between groups (FIG. 38K).

### Conclusions

Although extensive research has generally explored the importance of adjuvants and antigens in creating powerful new immunotherapies, to date, no body of work has explored the importance of structural presentation of multiple antigens within a specific construct and its role in eliciting a potent and desired immune response. Collectively, data provided herein show that antigen placement is as critical as antigen choice in vaccine efficacy. Indeed, in a specific embodiment, when altering the placement of MHC-I and -II restricted antigens in two compositionally nearly identical vaccines, the treatment benefit against tumors is dramatically changed; one vaccine was potent and the other was relatively ineffective. The origins of these differences may be due to antigen positioning affecting the pathway of processing that it undergoes in an immune cell, as well as its residence time in different cellular compartments. By changing the processing pathway and these kinetics of signaling, this affects the resulting immune response at the genetic, cellular, and organismal levels. An encapsulated MHC-II and a hybridized MHC-I restricted antigen upregulated genes specific for inflammatory responses, chemotaxis, and migration of key immune cells, which together influence immune cell activity. These structurally-defined genetic differences translated through to immunological behavior upon repeated in vivo immunization, and ultimately define the tumor growth profiles in both a model E.G7-OVA lymphoma and clinically-relevant B16-F10 melanoma mouse tumor system. This is a key demonstration of the impact of vaccine antigen positioning across multiple cellular processes.

The present Example showed that the power to optimize antigen presentation to match a desired signaling profile is critical to generate potent vaccines, where small vaccine changes in antigen placement significantly elevated cell-cell communication, cross-talk, and cell synergy.

### Materials and Methods

Materials and Animals: Unless otherwise noted, all reagents were purchased commercially and used as received. Oligonucleotides were synthesized as described below. Peptides were purchased from Genscript or Northwestern's Peptide Synthesis core. Chemicals were purchased from suppliers listed in parentheses. C57BL/6 mice and C57BL/6-Tg(TcraTcrb)1100Mjb/J (OT-1, 003831) female mice, age 8-12 weeks old, were purchased from Jackson Laboratory. Mice were used in accordance with all national and local guidelines and regulations and protocols performed were approved by the institutional animal use committee at Northwestern University (IUCAC). E.G7-OVA and B16-F10 cells were purchased from ATCC. Antibodies were purchased and clones are provided in Table 3.

TABLE 3
Antibody Chart for those used in this Example.
Flow Cytometry Phenotyping Antibodies (Mouse)

| | Fluorochrome | | | Catalog |
|---|---|---|---|---|
| Antibody | or Label | Clone | Company | No. |
| CD4 | PE | GK1.5 | BD | 557308 |
| | APC | GK1.5 | BioLegend | 100412 |
| CD8 | APC | 53-6.7 | BD | 553035 |
| | PE-Cy7 | 53-6.7 | BioLegend | 100708 |
| CD11c | Biotin | N418 | BioLegend | 117304 |
| CD19 | FITC | 6D5 | BioLegend | 115506 |
| | PE-Cy7 | 6D5 | BioLegend | 115520 |
| CD44 | BV450 | IM7 | BD | 560451 |
| CD62L | PE-Cy7 | MEL-14 | BD | 560516 |
| CD69 | AF700 | H1.2F3 | BD | 561238 |
| CD80 | PE | 16-10A1 | BioLegend | 104708 |
| CD86 | FITC | GL1 | BioLegend | 105006 |
| CD107 | FITC | 1D4B | BioLegend | 121606 |
| IFN-γ | PE-Cy7 | XMG1.2 | BioLegend | 505826 |
| DimerX I H-2Kb | PE | | BD | 552944 |
| (OVA1) | | | | |
| OVA2 tetramer | APC | | ProImmune | Custom |
| | | | | order |
| M27 pentamer | PE | | ProImmune | Custom |
| | | | | order |
| M30 tetramer | APC | | ProImmune | Custom |
| | | | | order |
| Live/Dead | UV | | Invitrogen | L34962 |

Oligonucleotide Synthesis and Purification: Oligonucleotides were synthesized on an ABI 394 synthesizer using standard phosphoramidite chemistry with phosphate or phosphorothioate backbones, as indicated (Table 1). Following synthesis, the strands were deprotected using a 1:1 solution of 37% ammonium hydroxide/40% methylamine at 55° C. for 35 min, unless they contained a dye, in which case they were deprotected using 37% ammonium hydroxide at room temperature (RT) overnight. The strands were then purified using a C18 or C4 (for strands containing dye or cholesterol) column on reverse phase HPLC, and the peaks were collected as fractions. The dimethoxytrityl (DMT) group was removed from the product strands by incubation in 20% aqueous acetic acid at RT for 1 h, followed by three washes with ethyl acetate to remove DMT. The final product was lyophilized and resuspended in deionized water (diH₂O). The concentration was measured using UV-vis absorption at 260 nm with extinction coefficients calculated through the IDT OligoAnalyzer online tool (listed in Table 1).

Oligonucleotide-peptide Conjugate Synthesis and Purification: Thiol-functionalized oligonucleotides in diH₂O were reduced to generate a free thiol for future reactions. Reduction was done using dithiothreitol (100 mM, DTT, Sigma) dissolved in phosphate buffered saline (PBS) pH 8.5 at a final concentration of 100 mM at RT for 45 min. This solution was washed in a 3 kDa molecular weight cut off (MWCO) spin filter (Amicon) at least three times with diH₂O. For OVA peptide conjugates, peptide was purchased on resin and washed three times each with dimethylformamide (DMF) and acetone before reacting 5 pmol at RT overnight with a solution of succinimidyl 2-(2-pyridyldithio)ethyl carbonate (SDEC, made using previous protocols 39) dissolved in DMF (10 equivalents with respect to the initial peptide loading on the solid support), with N,N-diisopropylethylamine (5 equivalents). The beads were subsequently washed three times with DMF and acetone each and dried in air before being deprotected with 95% Trifluoroacetic acid (2.5% Triisopropyl silane, 2.5% diH2O) for 1 h at RT. The TFA was blown off using nitrogen, and the beads were redissolved in DMF and filtered through glass wool. The peptide product was precipitated by adding approximately 5-6 times diethyl ether and was left at −20° C. for 1-2 h to further precipitate. The solution was centrifuged (2,000×g, 3 min) to pellet the peptide, which was collected, dried, and dissolved in DMF. Reduced DNA (0.5 pmol) was reacted overnight at RT with the dissolved peptide (5 pmol) in 70-75% DMF in water for a total volume of reaction of approximately 1.5 mL. For MART-1 peptide conjugates, the peptides were activated using 2,2′-dithiodipyridine (150 pmol) dissolved in 10 equivalents DMF under gentle agitation for 30 min at RT. The activated peptide was then washed three times in diethyl ether, pelleted by centrifugation (2,000×g, 3 min), and allowed to dry. Reduced DNA (0.3 pmol) was reacted overnight at RT with the dissolved peptide (1.5 pmol) in approximately 70% DMF in water for a total volume of reaction of approximately 1.5 mL.

Following conjugation of the peptide, the solutions were centrifuged at 17,000×g for 2 min to pellet any crashed out peptide, and the supernatant was transferred to 3 kDa MWCO spin filters for 3-4 washes with diH₂O. The volume was concentrated to <500 μL, and the solutions were purified by preparatory scale denaturing (8 M urea) 15% PAGE gels (no more than 0.5 μmol by DNA loaded onto a single gel). The gels were run for 30 min at 175 V, then approximately 3 h at 350 V, and subsequently imaged using a UV lamp to cut out desired bands. Cut-out gel bands were crushed, and the product was collected by three washes with 1× Tris/Borate/EDTA (TBE) buffer every approximately 4 h. The product mass was confirmed by electrospray ionization mass spectrometry (ESI-MS), and the concentrations were measured by UV-vis at 260 nm assuming an extinction coefficient of the DNA.

SNA Synthesis: SNAs were synthesized as reported previously with slight modifications.^{16, 40} Briefly, dried lipid films of 50 mg of 1,2-dioleoyl-sn-glycero-3-phosphocholine (DOPC, Avanti Polar Lipids) were hydrated with 3-4 mL of PBS or PBS-containing peptide, for encapsulated liposomes. (Note: solutions containing peptide included, OVA1: 1 mg/mL dissolved in PBS containing approximately 100 μL 1 M NaOH; OVA2: 1 mg/mL dissolved in PBS containing approximately 60 μL 1 M NaOH; M27: 2.25 mg/mL dissolved in PBS containing approximately 60 μL 1M NaOH; M30: 1 mg/mL dissolved in PBS containing approximately 100 μL 1 M NaOH) The solutions were subjected to 20 freeze-thaws in liquid nitrogen and then sonication at 37-40° C. The liposomes were extruded using sequential high-pressure extrusion (Northern Lipids Inc.) using polycarbonate filters with pore sizes of 200, 100, 80, and 50 nm; liposomes were passed through each pore size three times. Following extrusion, the liposomes were concentrated down to approximately 2-3 mL using 100 kDa MWCO spin filters and dialyzed overnight against 3.5 L of PBS to remove unencapsulated peptide. The liposome concentration was determined using a phosphatidylcholine (PC) assay kit (Sigma, MAK049-1 KT), assuming a 50-nm liposome contains 18,140 lipids per liposome.²⁴ Peptide concentration, if liposomes encapsulated peptide, was determined using a Pierce™ fluorescence assay kit (Thermofisher, 23290) adding 1% sodium dodecyl sulfate (SDS) to rupture liposomes and release peptide for quantification, and using peptide dissolved in 1% SDS as a standard curve. The loading of peptide per liposome was calculated by dividing the peptide concentration over the liposome concentration. The amounts of each antigen per particle ranged from ca. 25-40 depending on the encapsulation yield of each batch, which was tuned using different starting concentrations.

Purified oligonucleotide-peptide conjugates were mixed in a 1:1 molar ratio with complementary 3′-cholesterol-terminated CpG DNA and centrivapped overnight. The next day, approximately 20-40 μL of duplex buffer (IDT) was added and the solution was slow-cooled to duplex the strands following the program: 70° C. for 10 min, 23° C. for 1.5 h, 4° C. for >1 h. The duplex was added to a solution of synthesized liposomes at an equimolar amount to the peptide encapsulated within the liposome. To obtain the maximum 75 strands per liposome, the remaining space was filled with 3′cholesterol terminated T20 DNA. This mixture was incubated at 37° C. overnight and subsequently stored at 4° C.

Cell culture: All cells were maintained at 37° C. in a 5% CO₂ incubator. E.G7-OVA cells and DCs were cultured with RPMI 1640 media (Gibco, 11875093) containing 10% heat-inactivated fetal bovine serum (HI-FBS) and 1% Penicillin-Streptomycin, referred to herein as RPMI+/+. B16-F10 were handled using DMEM media (Gibco, 11965092) containing 10% HI-FBS and 1% Penicillin-Streptomycin.

Bone marrow-derived dendritic cell (BMDC) collection: Bone marrow cells were collected from mice following a previous protocol; 39 briefly, red blood cells were lysed with 2-3 mL of ACK lysis buffer (Gibco, A1049201) for approximately 4 min and plated on 10-cm² cell culture dishes with 40 ng/mL GM-CSF (BioLegend, 576304) for 5-7 days prior to use to differentiate DCs from the population.

BMDC Activation and Cross-priming of T cells In vitro: The cells were collected from 10-cm² cell culture dishes, and DCs were isolated from the mixture using a magnetic biotin positive selection kit (Stemcell Technologies, 17665). A CD11c⁺ biotin-labeled antibody was used to select DCs (BioLegend) and, after separation, purified DCs were counted using a Vi-CELL BLU Cell Viability Analyzer. For DC activation, 6×10⁴ DCs were cultured with SNA treatment in a final volume of 200 μL. After 22 h in an incubator, the cells were washed with PBS to end treatment, stained for 15 min at 4° C. using 0.5 μL of each antibody per tube (L/D, CD11c, CD86 and CD80), washed with PBS, and fixed with 100 μL of fixation buffer (BioLegend, 420801). To assess T cell specificity and activation, 1.6×10⁵ purified DCs were pulsed with SNA treatment for 30 min in the incubator in a final volume of 200 μL. After the 30 min pulse, the cells were washed twice with RPMI+/+ to remove any residual SNAs from the cell solution, and the cells were resuspended in 500 μL RPMI+/+. Concurrently, splenocytes were isolated from a naïve mouse. After dissociation of the spleen and lysis of the red blood cells, the cells were counted and resuspended to a concentration of 3×10⁶ cells/mL in warmed RPMI+/+, and 100 μL of this cell solution was transferred to each well in a 96-well round bottom plate. To each well, 100 μL of treated DCs (3.3×10⁴ cells) were added so that the ratio of DC:splenocytes was 1:9. The cells were co-cultured for three days in the incubator, after which cells were washed with PBS and stained following the manufacturer's instructions for either the DimerX Mouse H-2Kb:Ig Fusion Protein (BD, 552944) or OVA2 Tetramer (Prolmmune). Staining antibodies in addition to the peptide-specific TCR markers included L/D, either CD8 or CD4, CD19, and CD69. After staining, the cells were fixed with 100 μL of fixation buffer. To assess T cell proliferation, 2.6×10⁵ of purified DCs were pulsed with SNA treatment for 30 min in the incubator in a final volume of 200 μL. After the 30 min pulse, the cells were washed twice with RPMI+/+ to remove any residual SNA from the cell solution, and the cells were resuspended in 266.6 μL RPMI+/+. Concurrently, splenocytes were isolated from a C57BL/6-Tg(TcraTcrb)1100Mjb/J (OT-1) mouse (Jackson, 003831). After dissociation of the spleen and lysis of the red blood cells, the cells were counted and resuspended to a concentration of 4×10⁷ cells/mL in PBS for staining with Cell Proliferation Dye eFluor™ 450 (eBioscience, 65-0842-85), following the manufacturer's instructions. After staining, the cells were washed, counted, and resuspended in RPMI+/+ to a concentration of 3×10⁶ cells/mL; 100 μL of this cell solution was transferred to each well in a 96-well round bottom plate. To each well, 33.3 μL of treated DCs (3.3×10⁴ cells) were added so that the ratio of DC:splenocytes was 1:9, and each well was brought up to 200 μL final volume with media. The cells were co-cultured for three days in the incubator, after which cells were washed with PBS, stained for CD8 (0.5 μL antibody per tube), washed, and immediately analyzed on flow cytometry using dilution of eFluor™ 450 as a measure of T cell proliferation. All samples were analyzed using a BD A3 Symphony flow cytometer with data analyzed on FlowJo.

In Vivo Immunization to Measure Five-week Built-up Immune Responses: Female C57BL/6 mice were subcutaneously immunized fortnightly three times with different treatments. Treatments included: simple mixture (admix, 6 nmol each peptide and 6 nmol CpG 1826 DNA) or either DA-SNA (6 nmol by each OVA peptide and CpG 1826). Volume of treatment injected was kept at 100 μL. One week after the final immunization, mice were sacrificed, and spleens were harvested for subsequent immune assessment.

Harvest Procedure: Removed spleens were collected and held temporarily in 3-5 mL of RPMI+/+ until all spleens were collected, when they were passed through a 70 μm cell strainer with a constant flow of PBS. The cells were centrifuged at 1,200 rpm for 5 min, after which supernatant was removed, and the cells were resuspended in 2-3 mL ACK lysing buffer (Gibco, A1049201) for 4 min. To dilute the lysing buffer, PBS was then added to a final volume of 30 mL, and the cells were counted prior to centrifugation to resuspend in RPMI+/+ media at a concentration of 1×10⁸ cells mL¹.

IFN-γCytokine Production: T cells were restimulated ex vivo to assess antigen-specific intracellular IFN-γ production. 4×10⁶ splenocytes were cultured for 4 h at 37° C. in a 5% CO₂ incubator with 450 μL of RPMI+/+ media containing: either OVA1 or OVA2 peptide (10 μg/mL), monensin (2 μM), brefeldin A (5 μg/mL), and CD107a antibody (0.5 μL). After the 4 h incubation, cells were centrifuged at 1,200 rpm for 5 min, aspirated, and washed with 600 μL of PBS, prior to 15 min of staining with surface antibodies (0.5 μL per sample each of: L/D, CD8, CD4) at 4° C. The cells were washed with 600 μL PBS, centrifuged at 1,200 rpm for 5 min, aspirated, and resuspended in 100 μL of Cytofix Fixation and Permeabilization solution (BD, 554722) for 20 min at 4° C. After, the cells were washed with 600 μL of Perm/Wash Buffer (BD, 554723), centrifuged at 1,200 rpm for 5 min, aspirated, and resuspended in 100 μL of Perm/Wash Buffer containing the intracellular antibody IFN-γ (0.5 μL per sample). The samples were stored at 4° C. prior to flow cytometry analysis.

T cell Memory Phenotyping: T cells were assessed for effector memory phenotype. 3×10⁶ splenocytes were washed with 600 μL PBS, and stained for 15 min with surface antibodies (0.5 μL per sample each of: L/D, CD8, CD4, CD44, and CD62L) at 4° C. The cells were washed with 600 μL PBS, centrifuged at 1,200 rpm for 5 min, aspirated, and resuspended in 100 μL of Fixation buffer (BioLegend, 420801), and stored at 4° C. prior to flow cytometry analysis.

ELISpot Assay: ELISpot analysis was performed using the commercially available Mouse INF-7 ELISPOT Set (BD, 551083) following the manufacturer's instructions. Briefly, the provided clean plate was coated overnight at 4° C. with capture antibody. After, the plate was washed with RPMI+/+ media and then blocked for 2 h at room temperature with 200 μL of RPMI+/+ media. The blocking buffer was removed by pipetting, being mindful not to let wells dry out, and quickly replaced with 2×10⁵ splenocytes in 100 μL RPMI+/+. To each well, an additional 100 μL of either antigen, non-specific peptide, media (negative control), or positive control solutions were added (antigen and non-specific peptide were added to a final concentration of 5 μg/mL; positive control was prepared as a mixture of anti-CD3 and anti-CD28 antibodies at a final concentration of 2 μg/mL each). The solutions were left in an incubator at 37° C. in 5% CO2 for 48 h. After this incubation, the plate was washed, and detection antibody, enzyme conjugate, and chromogenic substrate were added according to the manufacturer's instructions. The dried plate was imaged and analyzed using a CTL Immunospot imager.

Bulk RNA Sequencing: CD4⁺ and CD8⁺ T cells were isolated from whole splenocytes from individual treatment groups using magnetic positive selection kits (Stemcell Technologies, 18952 and 18953). From these isolated cell populations, RNA extraction was performed using an RNeasy® Plus Mini Kit (Qiagen) in combination with QIAshredders (Qiagen) following manufacturer's specifications. RNA concentration was quantified using a NanoDrop 8000 (Thermo Scientific), and RNA samples were stored in −80° C. until further use. Sequencing was conducted at the Northwestern University NUSeq Core Facility. Briefly, total RNA examples were checked for quality using RNA integrity numbers (RINs) generated from Agilent Bioanalyzer 2100. RNA quantity was confirmed with a Qubit fluorometer. The Illumina TruSeq Stranded mRNA Library Preparation Kit was used to prepare sequencing libraries from 125 ng of high-quality RNA samples (RIN>7). The kit procedure, including mRNA purification and fragmentation, cDNA synthesis, 3′ end adenylation, Illumina adapter ligation, library PCR amplification and validation, was performed without modifications. Libraries were sequenced using an Illumina HiSeq 4000 sequencer to generate 50 bp single reads at the depth of 20-25 million reads per sample. The quality of reads, in FASTQ format, was evaluated using FastQC. Reads were trimmed to remove Illumina adapters from the 3′ ends using cutadapt.⁴¹ Trimmed reads were aligned to the Mus musculus genome (mm10) using STAR.⁴² Read counts for each gene were calculated using htseq-count⁴³ in conjunction with a gene annotation file for mm10 obtained from Ensembl (http://useast.ensembl.org/index.html). Normalization and differential expression were calculated using DESeq2 that employed the Wald test.⁴⁴ The cutoff for determining significantly differentially expressed genes was an FDR-adjusted p-value less than 0.05 using the Benjamini-Hochberg method.

Gene set enrichment analysis (GSEA): GSEA45 was performed to understand whether differentially expressed genes were connected to differentially enriched pathways. Genes detected with RNA sequencing were ranked based on log₁₀-transformed nominal P values obtained from DeSeq2 analysis, and were compared to naïve T cells. Pathway enrichment analysis was performed using the GSEA software (v4.0.3) and following the protocol of Reimand, et al.⁴⁶ Gene sets were obtained from the Molecular Signatures Database and included Reactome, KEGG. The ranked list was remapped using a CHIP platform from the Molecular Signatures Database that used the Mouse Gene Symbol to remap to Human Orthologs (v7.1). A term was defined as differentially enriched if it had an FDR<0.05. A subset of strongly enriched pathways was selected for visualization in R using pheatmap package (v1.0.12). This selection included all pathways with an FDR<0.05 and were of relevance to immune responses in CD8⁺ and CD4⁺ T cells.

Gene expression profiles: Genes whose expression were significantly altered in both SNA treatment groups, as defined by FDR p-value <0.05, were selected for visualization as heatmaps. Gene expression scores in FPKM were converted to z-scores across treatment groups and gene expression values clustered using K-means clustering. Pairwise combinations were performed between two conditions of interest, setting naïve CD4⁺ or CD8⁺ T cells as controls. Genes up or down regulated between groups as defined by FDR p-value <0.05 and log 2 fold-change of >0.5 (up-regulated) or log₂ fold-change <0.05 (down-regulated) were visualized using a volcano plot. In vivo efficacy studies: Female C57BL/6 mice aged 8-12 weeks were acquired from The Jackson Laboratory. Tumor inoculation was performed by subcutaneously (s.c.) injecting animals with either 5×10⁵ E.G7-OVA or 105 B16-F10 cells in the right flank. Immunizations were administered at a dose of either 6 nmol (OVA1/2) or 9 nmol (M27/30) of each antigen and CpG by s.c. injection in the abdomen. Immunizations were administered as listed in the treatment schedule provided in respective figures by s.c. injection in the abdomen. For combination therapy with the immune checkpoint inhibitor anti-PD-1, mice were administered 100 μg anti-mouse PD-1 (clone RMP1-14, BioXCell) via intraperitoneal injection. Tumor growth was measured on pre-determined days and volume was calculated using the following equation: tumor volume=length×width²×0.5. Animals were euthanized when tumor volumes reached either 2,000 mm³ (E.G7-OVA) or 1,500 mm³ (B16-F10) or when the animal became moribund.

Immunoactivation of PBMCs: For collection of peripheral blood mononuclear cells (PBMCs), animals were inoculated with cancer cells as described above. Treatment was performed following the same schedule and animals were euthanized on day 15 (E.G7-OVA) or day 17 (B16-F10). Blood was collected via cardiac puncture into EDTA-lined collection tubes (BD) and briefly mixed by inverting. Red blood cells were lysed using ACK lysing buffer (Gibco) and washed, and the remaining cells were subsequently stained using the methods described above with antibodies for L/D, CD4, CD8, CD19, CD44, and CD62L.

Whole organ Immune Assessment: Tumor weights and splenocyte evaluation was performed on C57BL/6 mice bearing an E.G7-OVA tumor in the right flank. Three days after tumor inoculation, the first immunization was administered followed by an additional dose 7 days later (day 10). The tumors and spleens were excised from the animals at day 15 and subsequently analyzed. To generate single-cell splenocyte solutions, the spleens were mechanically forced through a 70 μm cell strainer while maintaining hydration in PBS solution. The cells were subsequently centrifuged at 1200 rpm for 5 min. The pellet was then resuspended in ACK lysing buffer (Thermo) for 4 min to lyse red blood cells and subsequently neutralized in PBS prior to centrifugation. Following centrifugation, the cells were labeled using the following antibodies: CD4, CD8, and CD19.

Statistical Analysis: Statistics were calculated using GraphPad Prism 8 software, and specific statistical analyses used are highlighted in the respective figure captions. Comparisons between multiple groups were analyzed with a one-way ANOVA with a Šidák or Tukey's multiple comparison test, or a Welch ANOVA with a Dunnett multiple comparison test due to the lack of assumptions that could be made based on large differences in standard deviation between groups. Statistics for animal survival were calculated using a Log-rank test. Outliers for FIG. 35E-F and FIG. 38H,J were identified using the ROUT method with a Q set to 10% or 1%, respectively. For all cases, p-value was depicted as follows: *p<0.05; **p<0.01; ***p<0.001; ****p<0.0001; n.s. indicated that no significance was determined and n.d. indicates values were not detected. The allocation of animals to each group, administered immunizations, and measurements for tumor studies were performed blind. Values in graphs are depicted as the mean±s.e.m., and this, as well as sample size, is indicated in the respective figure caption.

### Example 3

This Example describes experiments using SNA constructs according to the disclosure in a colon cancer model.

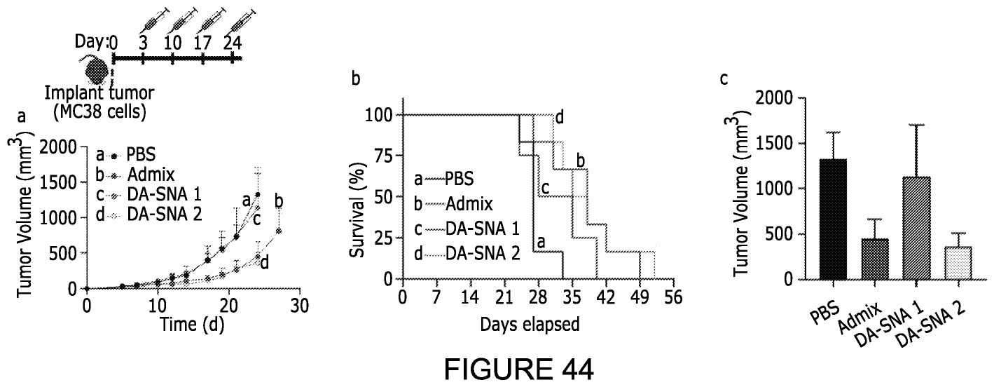

Female C57BL/6 mice aged 8-12 weeks were acquired from The Jackson Laboratory. Tumor inoculation was performed by subcutaneously (s.c.) injecting animals with 5×10⁵ MC38 colon cancer cells in the right flank. Immunizations were administered at a dose of either 6 nmol (Adpgk1/2) of each antigen and CpG by s.c. injection in the abdomen. Immunizations were administered as listed in the treatment schedule provided by s.c. injection in the abdomen. Tumor growth was measured on pre-determined days and volume was calculated using the following equation: tumor volume=length×width²×0.5. Animals were euthanized when tumor volumes reached either 2,000 mm³ (MC38) or when the animal became moribund. See FIG. 44.

### REFERENCES

1. Domingues, B., Lopes, J. M., Soares, P. & Populo, H. Melanoma treatment in review. Immunotargets Ther 7, 35-49 (2018).
2. Peled, N., Oton, A. B., Hirsch, F. R. & Bunn, P. MAGE A3 antigen-specific cancer immunotherapeutic. Immunotherapy 1, 19-25 (2009).
3. Panelli, M. C. et al. Phase 1 study in patients with metastatic melanoma of immunization with dendritic cells presenting epitopes derived from the melanoma-associated antigens MART-1 and gp100. J Immunother 23, 487-498 (2000).
4. Bhardwaj, N. et al. Flt3 ligand augments immune responses to anti-DEC-205-NY-ESO-1 vaccine through expansion of dendritic cell subsets. Nature Cancer 1, 1204-1217 (2020).
5. Alexandrov, L. B. et al. Signatures of mutational processes in human cancer. Nature 500, 415-421 (2013).
6. Slingluff, C. L., Jr. The present and future of peptide vaccines for cancer: single or multiple, long or short, alone or in combination? Cancer J 17, 343-350 (2011).
7. Beatty, G. L. & Gladney, W. L. Immune escape mechanisms as a guide for cancer immunotherapy. Clin Cancer Res 21, 687-692 (2015).
8. Ott, P. A. et al. An immunogenic personal neoantigen vaccine for patients with melanoma. Nature 547, 217-221 (2017).
9. Kenter, G. G. et al. Vaccination against HPV-16 oncoproteins for vulvar intraepithelial neoplasia. N Engl J Med 361, 1838-1847 (2009).
10. Melief, C. J. & van der Burg, S. H. Immunotherapy of established (pre)malignant disease by synthetic long peptide vaccines. Nat Rev Cancer 8, 351-360 (2008).
11. Melief, C. J. in Oncoimmunology: A Practical Guide for Cancer Immunotherapy. (eds. L. Zitvogel & G. Kroemer) 249-261 (Springer International Publishing, 2018).
12. Shirai, M. et al. Helper-cytotoxic T lymphocyte (CTL) determinant linkage required for priming of anti-HIV CD8+ CTL in vivo with peptide vaccine constructs. J Immunol 152, 549-556 (1994).
13. Lynn, G. M. et al. Peptide-TLR-7/8a conjugate vaccines chemically programmed for nanoparticle self-assembly enhance CD8 T-cell immunity to tumor antigens. Nat Biotechnol 38, 320-332 (2020).
14. Kuai, R., Ochyl, L. J., Bahjat, K. S., Schwendeman, A. & Moon, J. J. Designer vaccine nanodiscs for personalized cancer immunotherapy. Nat Mater 16, 489-496 (2017).
15. Scott, E. A. et al. Dendritic cell activation and T cell priming with adjuvant- and antigen-loaded oxidation-sensitive polymersomes. Biomaterials 33, 6211-6219 (2012).
16. Wang, S. et al. Rational vaccinology with spherical nucleic acids. Proc Natl Acad Sci USA 116, 10473-10481 (2019).
17. Ostroumov, D., Fekete-Drimusz, N., Saborowski, M., Kuhnel, F. & Woller, N. CD4 and CD8 T lymphocyte interplay in controlling tumor growth. Cell Mol Life Sci 75, 689-713 (2018).
18. Shankaran, V. et al. IFNgamma and lymphocytes prevent primary tumour development and shape tumour immunogenicity. Nature 410, 1107-1111 (2001).
19. Church, S. E., Jensen, S. M., Antony, P. A., Restifo, N. P. & Fox, B. A. Tumor-specific CD4+ T cells maintain effector and memory tumor-specific CD8+ T cells. Eur J Immunol 44, 69-79 (2014).
20. Marzo, A. L., Lake, R. A., Robinson, B. W. & Scott, B. T-cell receptor transgenic analysis of tumor-specific CD8 and CD4 responses in the eradication of solid tumors. Cancer Res 59, 1071-1079 (1999).
21. Okada, K. et al. Interactions between autologous CD4+ and CD8+ T lymphocytes and human squamous cell carcinoma of the head and neck. Cell Immunol 177, 35-48(1997).
22. Schirrmacher, V., Schild, H. J., Guckel, B. & von Hoegen, P. Tumour-specific CTL response requiring interactions of four different cell types and recognition of MHC class I and class II restricted tumour antigens. Immunol Cell Biol 71 (Pt 4), 311-326 (1993).
23. Bos, R. & Sherman, L. A. CD4+ T-cell help in the tumor milieu is required for recruitment and cytolytic function of CD8+ T lymphocytes. Cancer Res 70, 8368-8377 (2010).
24. Banga, R. J., Chernyak, N., Narayan, S. P., Nguyen, S. T. & Mirkin, C. A. Liposomal spherical nucleic acids. J Am Chem Soc 136, 9866-9869 (2014).
25. Choi, C. H., Hao, L., Narayan, S. P., Auyeung, E. & Mirkin, C. A. Mechanism for the endocytosis of spherical nucleic acid nanoparticle conjugates. Proc Natl Acad Sci USA 110, 7625-7630 (2013).
26. Rosi, N. L. et al. Oligonucleotide-modified gold nanoparticles for intracellular gene regulation. Science 312, 1027-1030 (2006).
27. Radovic-Moreno, A. F. et al. Immunomodulatory spherical nucleic acids. Proc Natl Acad Sci USA 112, 3892-3897 (2015).
28. Cutler, J. I., Auyeung, E. & Mirkin, C. A. Spherical nucleic acids. J Am Chem Soc 134, 1376-1391 (2012).
29. Sinegra, A. J., Evangelopoulos, M., Park, J., Huang, Z. & Mirkin, C. A. Lipid Nanoparticle Spherical Nucleic Acids for Intracellular DNA and RNA Delivery. Nano Lett (2021).
30. Yamankurt, G. et al. Exploration of the nanomedicine-design space with high-throughput screening and machine learning. Nat Biomed Eng 3, 318-327 (2019).
31. Hoyer, S. et al. Concurrent interaction of DCs with CD4(+) and CD8(+) T cells improves secondary CTL expansion: It takes three to tango. Eur J Immunol 44, 3543-3559 (2014).
32. Mailliard, R. B. et al. Complementary dendritic cell-activating function of CD8+ and CD4+ T cells: helper role of CD8+ T cells in the development of T helper type 1 responses. J Exp Med 195, 473-483 (2002).
33. Seder, R. A., Darrah, P. A. & Roederer, M. T-cell quality in memory and protection: implications for vaccine design. Nat Rev Immunol 8, 247-258 (2008).
34. Moore, M. W., Carbone, F. R. & Bevan, M. J. Introduction of soluble protein into the class I pathway of antigen processing and presentation. Cell 54, 777-785 (1988).
35. Overwijk, W. W. & Restifo, N. P. B16 as a mouse model for human melanoma. Curr Protoc Immunol Chapter 20, Unit 20 21 (2001).
36. Kreiter, S. et al. Mutant MHC class II epitopes drive therapeutic immune responses to cancer. Nature 520, 692-696 (2015).
37. van Rooij, N. et al. Tumor exome analysis reveals neoantigen-specific T-cell reactivity in an ipilimumab-responsive melanoma. J Clin Oncol 31, e439-442 (2013).
38. Topalian, S. L. et al. Safety, activity, and immune correlates of anti-PD-1 antibody in cancer. N Engl J Med 366, 2443-2454 (2012).
39. Skakuj, K. et al. Conjugation Chemistry-Dependent T-Cell Activation with Spherical Nucleic Acids. Journal of the American Chemical Society 140, 1227-1230 (2018).
40. Qin, L. et al. Development of Spherical Nucleic Acids for Prostate Cancer Immunotherapy. Front Immunol 11, 1333 (2020).
41. Martin, M. Cutadapt removes adapter sequences from high-throughput sequencing reads. 2011 17, 3 (2011).
42. Dobin, A. et al. STAR: ultrafast universal RNA-seq aligner. Bioinformatics 29, 15-21 (2013).
43. Anders, S., Pyl, P. T. & Huber, W. HTSeq—a Python framework to work with high-throughput sequencing data. Bioinformatics 31, 166-169 (2015).
44. Love, M. I., Huber, W. & Anders, S. Moderated estimation of fold change and dispersion for RNA-seq data with DESeq2. Genome Biol 15, 550 (2014).
45. Subramanian, A. et al. Gene set enrichment analysis: a knowledge-based approach for interpreting genome-wide expression profiles. Proc Natl Acad Sci USA 102, 15545-15550 (2005).
46. Reimand, J. et al. Pathway enrichment analysis and visualization of omics data using g:Profiler, GSEA, Cytoscape and EnrichmentMap. Nat Protoc 14, 482-517 (2019).

What is claimed is:

1. A spherical nucleic acid (SNA) comprising:
(a) a nanoparticle core;
(b) a shell of oligonucleotides attached to the external surface of the nanoparticle core, the shell of oligonucleotides comprising one or more immunostimulatory oligonucleotides; and
(c) a first antigen that is a major histocompatibility complex type I (MHC-I) antigen, and a second antigen that is a major histocompatibility complex type II (MHC-II) antigen.

2. The SNA of claim 1, wherein the first antigen is encapsulated in the nanoparticle core.

3. The SNA of claim 1 or claim 2, wherein the second antigen is attached to one or more oligonucleotides in the shell of oligonucleotides through a linker.

4. The SNA of claim 3, wherein the second antigen is attached through the linker to an oligonucleotide in the shell of oligonucleotides that is attached to the nanoparticle core.

5. The SNA of claim 3, wherein the second antigen is attached through the linker to an oligonucleotide that is hybridized to an oligonucleotide in the shell of oligonucleotides that is attached to the nanoparticle core.

6. The SNA of claim 1 or claim 2, wherein the second antigen is attached to the external surface of the nanoparticle core through a linker.

7. The SNA of claim 1 or claim 2, wherein the second antigen is encapsulated in the nanoparticle core.

8. The SNA of any one of claim 1 or 3-7, wherein the first antigen is attached to one or more oligonucleotides in the shell of oligonucleotides through a linker.

9. The SNA of claim 8, wherein the first antigen is attached through the linker to an oligonucleotide in the shell of oligonucleotides that is attached to the nanoparticle core.

10. The SNA of claim 8, wherein the first antigen is attached through the linker to an oligonucleotide that is hybridized to an oligonucleotide in the shell of oligonucleotides that is attached to the nanoparticle core.

11. The SNA of any one of claim 1 or 3-7, wherein the first antigen is attached to the external surface of the nanoparticle core through a linker.

12. The SNA of any one of claims 1-11, comprising a third antigen that is a major histocompatibility complex type I (MHC-I) antigen.

13. The SNA of any one of claims 1-12, comprising a fourth antigen that is a major histocompatibility complex type II (MHC-II) antigen.

14. The SNA of claim 12 or claim 13, wherein the third antigen is encapsulated in the nanoparticle core.

15. The SNA of claim 13 or claim 14, wherein the fourth antigen is attached to one or more oligonucleotides in the shell of oligonucleotides through a linker.

16. The SNA of claim 15, wherein the fourth antigen is attached through the linker to an oligonucleotide in the shell of oligonucleotides that is attached to the nanoparticle core.

17. The SNA of claim 15, wherein the fourth antigen is attached through the linker to an oligonucleotide that is hybridized to an oligonucleotide in the shell of oligonucleotides that is attached to the nanoparticle core.

18. The SNA of claim 13 or claim 14, wherein the fourth antigen is attached to the external surface of the nanoparticle core through a linker.

19. The SNA of any one of claims 12, 13, or 15-18, wherein the third antigen is attached to one or more oligonucleotides in the shell of oligonucleotides through a linker.

20. The SNA of claim 19, wherein the third antigen is attached through the linker to an oligonucleotide in the shell of oligonucleotides that is attached to the nanoparticle core.

21. The SNA of claim 19, wherein the third antigen is attached through the linker to an oligonucleotide that is hybridized to an oligonucleotide in the shell of oligonucleotides that is attached to the nanoparticle core.

22. The SNA of any one of claims 12, 13, or 15-18, wherein the third antigen is attached to the external surface of the nanoparticle core through a linker.

23. The SNA of any one of claims 13, 14, or 19-22, wherein the fourth antigen is encapsulated in the nanoparticle core.

24. The SNA of any one of claims 12-23, wherein the first antigen and the third antigen are the same.

25. The SNA of any one of claims 12-23, wherein the first antigen and the third antigen are different.

26. The SNA of any one of claims 13-25, wherein the second antigen and the fourth antigen are the same.

27. The SNA of any one of claims 13-25, wherein the second antigen and the fourth antigen are different.

28. The SNA of any one of claims 1-27, wherein the MHC-I antigen is OVA₂₅₇₋₂₆₄ (OVA1) (SEQ ID NO: 7), GP100 (25-33) (SEQ ID NO: 11), TC-1 E6 (49-58) (SEQ ID NO: 12), TC-1 E7 (49-57) (SEQ ID NO: 13), PSMA (634-642) (SEQ ID NO: 14), SPAS-1 (SNC9-H8) (SEQ ID NO: 15), SIMS2 (237-245) (SEQ ID NO: 16), PAP (115-123) (SEQ ID NO: 17), B16 MART-1 (M27) (SEQ ID NO: 9), TRP-1 (252-260) (SEQ ID NO: 18), TRP-1 (252V260M) (SEQ ID NO: 19), TRP-1 (455-463) (SEQ ID NO: 20), TRP-1 (455A463M) (SEQ ID NO: 21), TRP-2 (180-188) (SEQ ID NO: 22), Melan-A/MART-(127-135), Tyrokinase(1-9), Tyrokinase(369-377D), MC38 Adpgk (SEQ ID NO: 23), Irgq-Minimum (SEQ ID NO: 24), Irgq-Long peptide (SEQ ID NO: 25), or a combination thereof.

29. The SNA of any one of claims 1-28, wherein the MHC-II antigen is OVA₃₂₃₋₃₃₉ (OVA2) (SEQ ID NO: 8), GP100: (46-58) (SEQ ID NO: 26), TC-1 E6 (43-57) (SEQ ID NO: 27), SIMS2 (240-254) (SEQ ID NO: 28), PAP (114-128) (SEQ ID NO: 29), B16 MART-1 (M30) (SEQ ID NO: 30), TRP-1 (113-127) (SEQ ID NO: 31), TRP-1 (106-130) (SEQ ID NO: 32), Li-Key (77-92) (SEQ ID NO: 33), Tyrosinase (56-70), GP100 (44-59), GP100 (167-189), Melan-A/MART-1(102-111) (SEQ ID NO: 34), Melan-A/MART-1(27-40) (SEQ ID NO: 35), Melan-A/MART-1(51-70) (SEQ ID NO: 36), Melan-A/MART-1(51-73) (SEQ ID NO: 37), Melan-A/MART-1(43-57) (SEQ ID NO: 38), or a combination thereof.

30. The SNA of any one of claims 1-29, wherein at least one of the one or more immunostimulatory oligonucleotides is a toll-like receptor (TLR) agonist.

31. The SNA of any one of claims 1-30, wherein each of the one or more immunostimulatory oligonucleotides is a toll-like receptor (TLR) agonist.

32. The SNA of claim 30 or claim 31, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

33. The SNA of any one of claims 30-32, wherein the TLR is TLR9.

34. The SNA of any one of claims 1-33, wherein the immunostimulatory oligonucleotide comprises a CpG nucleotide sequence.

35. The SNA of any one of claims 1-34, wherein one or more oligonucleotides in the shell of oligonucleotides comprises or consists of the sequence of 5′-TCCATGACGTTCCTGACGTT-3′ (SEQ ID NO: 39).

36. The SNA of any one of claims 1-35, wherein one or more oligonucleotides in the shell of oligonucleotides comprises or consists of the sequence of 5′-TCGTCGTTTTGTCGTTTTGTCGTT-3′ (SEQ ID NO: 40).

37. The SNA of any one of claims 1-36, wherein one or more oligonucleotides in the shell of oligonucleotides comprises or consists of the sequence of 5′-TCCATGACGTTCCTGACGTT(Spacer-18 (hexaethyleneglycol))₂Cholesterol-3′ (SEQ ID NO: 41).

38. The SNA of any one of claims 1-37, wherein one or more oligonucleotides in the shell of oligonucleotides comprises or consists of the sequence of 5′-TCGTCGTTTTGTCGTTTTGTCGTT(Spacer-18 (hexaethyleneglycol))₂Cholesterol-3′ (SEQ ID NO: 6).

39. The SNA of any one of claims 1-38, wherein at least about 10%, 20%, 30%, 40%, 50%, 60%, 70% 80%, 90%, 95%, or 100% of oligonucleotides in the shell of oligonucleotides is an immunostimulatory oligonucleotide.

40. The SNA of any one of claims 1-39, wherein the linker is a carbamate alkylene disulfide linker, a thiol linker, a disulfide linker, an amide alkylene disulfide linker, an amide alkylene thio-succinimidyl linker, or a combination thereof.

41. The SNA of any one of claims 1-40, wherein the nanoparticle core is a micelle, a liposome, a polymer, a lipid nanoparticle (LNP), or a combination thereof.

42. The SNA of claim 41, wherein the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), or chitosan.

43. The SNA of claim 42, wherein the polymer is poly(lactic-co-glycolic acid) (PLGA).

44. The SNA of any one of claims 1-43, wherein the nanoparticle core is a liposome.

45. The SNA of claim 44, wherein the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

46. The SNA of any one of claims 1-45, wherein one or more oligonucleotides in the shell of oligonucleotides is attached to the external surface of the nanoparticle core through a lipid anchor group.

47. The SNA of claim 46, wherein the lipid anchor group is attached to the 5′ end or the 3′ end of the one or more oligonucleotides.

48. The SNA of claim 46 or claim 47, wherein the lipid anchor group is tocopherol or cholesterol.

49. The SNA of any one of claims 1-48, wherein one or more oligonucleotides in the shell of oligonucleotides is modified on its 5′ end and/or 3′ end with dibenzocyclooctyl (DBCO).

50. The SNA of any one of claims 1-49, wherein one or more oligonucleotides in the shell of oligonucleotides is modified on its 5′ end and/or 3′ end with a thiol.

51. The SNA of any one of claims 1-50, wherein the shell of oligonucleotides comprises DNA oligonucleotides, RNA oligonucleotides, or a combination thereof.

52. The SNA of any one of claims 1-51, wherein the shell of oligonucleotides comprises DNA oligonucleotides and RNA oligonucleotides.

53. The SNA of any one of claims 1-52, wherein the shell of oligonucleotides comprises single-stranded DNA, double-stranded DNA, single-stranded RNA, double-stranded RNA, or a combination thereof.

54. The SNA of any one of claims 1-53, wherein one or more oligonucleotides in the shell of oligonucleotides is a modified oligonucleotide.

55. The SNA of any one of claims 1-54, wherein the shell of oligonucleotides comprises about 2 to about 200 oligonucleotides.

56. The SNA of any one of claims 1-55, wherein the shell of oligonucleotides comprises about 2 to about 100 oligonucleotides.

57. The SNA of any one of claims 1-55, wherein the shell of oligonucleotides comprises about 150 oligonucleotides.

58. The SNA of any one of claims 1-55, wherein the shell of oligonucleotides comprises about 200 oligonucleotides.

59. The SNA of claim 55 or claim 56, wherein the shell of oligonucleotides comprises about 10 to about 80 oligonucleotides.

60. The SNA of claim 55, claim 56, or claim 59, wherein the shell of oligonucleotides comprises about 75 oligonucleotides.

61. The SNA of any one of claims 1-60, wherein each oligonucleotide in the shell of oligonucleotides is about 5 to about 1000 nucleotides in length.

62. The SNA of claim 61, wherein each oligonucleotide in the shell of oligonucleotides is about 10 to about 50 nucleotides in length.

63. The SNA of claim 61 or claim 62, wherein each oligonucleotide in the shell of oligonucleotides is about 20 to about 30 nucleotides in length.

64. The SNA of any one of claims 1-63, wherein diameter of the SNA is about 1 nanometer (nm) to about 500 nm.

65. The SNA of any one of claims 1-64, wherein diameter of the SNA is less than or equal to about 80 nanometers.

66. The SNA of any one of claims 1-65, wherein diameter of the SNA is less than or equal to about 50 nanometers.

67. The SNA of any one of claims 1-66, wherein the shell of oligonucleotides comprises a targeting oligonucleotide, an inhibitory oligonucleotide, a non-targeting oligonucleotide, or a combination thereof.

68. The SNA of claim 67, wherein the inhibitory oligonucleotide is an antisense oligonucleotide, small interfering RNA (siRNA), an aptamer, a short hairpin RNA (shRNA), a DNAzyme, or an aptazyme.

69. A composition comprising a plurality of the SNA of any one of claims 1-68.

70. The composition of claim 69, wherein at least two SNAs in the plurality comprise a different nanoparticle core.

71. A pharmaceutical formulation comprising a plurality of the SNA of any one of claims 1-68, or the composition of claim 69 or claim 70, and a pharmaceutically acceptable carrier or diluent.

72. A vaccine comprising the SNA of any one of claims 1-68, the composition of claim 69 or claim 70, or the pharmaceutical formulation of claim 71.

73. The vaccine of claim 72, comprising an adjuvant.

74. An antigenic composition comprising the SNA of any one of claims 1-68 in a pharmaceutically acceptable carrier, diluent, stabilizer, or preservative, or the pharmaceutical formulation of claim 71, wherein the antigenic composition is capable of generating an immune response including antibody generation, cytotoxic T cell activation, helper T cell activation, or a protective immune response in a subject.

75. The antigenic composition of claim 74, wherein the immune response includes an antibody response.

76. The antigenic composition of claim 75, wherein the antibody response is a neutralizing antibody response or a protective antibody response.

77. A method of inhibiting expression of a gene product comprising hybridizing a polynucleotide encoding the gene product to the inhibitory oligonucleotide of claim 67 or claim 68, wherein hybridizing between the polynucleotide and the inhibitory oligonucleotide occurs over a length of the polynucleotide with a degree of complementarity sufficient to inhibit expression of the gene product.

78. The method of claim 77 wherein expression of the gene product is inhibited in vivo or in vitro.

79. A method of producing an immune response in a subject, comprising administering to the subject an effective amount of the SNA of any one of claims 1-68, the composition of claim 69 or claim 70, the pharmaceutical formulation of claim 71, the vaccine of claim 72 or claim 73, or the antigenic composition of any one of claims 74-76, thereby producing an immune response in the subject.

80. The method of claim 79, wherein the immune response includes an antibody response.

81. The method of claim 80, wherein the antibody response is a total antigen-specific antibody response.

82. The method of claim 80, wherein the antibody response is a neutralizing antibody response or a protective antibody response.

83. A method of immunizing a subject against one or more antigens comprising administering to the subject an effective amount of the SNA of any one of claims 1-68, the composition of claim 69 or claim 70, the pharmaceutical formulation of claim 71, the vaccine of claim 72 or 73, or the antigenic composition of any one of claims 74-76, thereby immunizing the subject against the one or more antigens.

84. The method of claim 83, wherein the composition or the vaccine is a cancer vaccine.

85. A method of treating a cancer comprising administering to a subject an effective amount of the SNA of any one of claims 1-68, the composition of claim 69 or claim 70, the pharmaceutical formulation of claim 71, the vaccine of claim 72 or 73, or the antigenic composition of any one of claims 74-76, thereby treating the cancer in the subject.

86. The method of claim 84 or claim 85, wherein the cancer is bladder cancer, breast cancer, cervical cancer, colon cancer, rectal cancer, endometrial cancer, glioblastoma, kidney cancer, leukemia, liver cancer, lung cancer, melanoma, lymphoma, non-Hodgkin lymphoma, osteocarcinoma, ovarian cancer, pancreatic cancer, prostate cancer, thyroid cancer, and human papilloma virus-induced cancer, or a combination thereof.

87. The method of claim 86, wherein the cancer is melanoma.

88. The method of claim 86 or claim 87, wherein the cancer is colon cancer.

89. The method of any one of claims 86-88, wherein the cancer is lymphoma.

90. The method of any one of claims 79-89, further comprising administering an additional agent.

91. The method of claim 90, wherein the additional agent is an anti-programmed cell death protein 1 (PD-1) antibody, an anti-programmed death-ligand 1 (PD-L1) antibody, a cytotoxic T lymphocyte antigen 4 (CTLA-4) antibody, a T cell immunoglobulin and ITIM domain (TIGIT) antibody, or a combination thereof.

92. The method of any one of claims 77-91, wherein the SNA is the SNA of claim 5.

93. The method of any one of claims 77-91, wherein the SNA is the SNA of claim 10.


SEQUENCE LISTING (41 sequences)

SEQ ID NO: 1 - DNA, 20 bp, Artificial Sequence
  Synthetic
  misc_feature (20)..(20): (SP18)2 CHOLESTEROL
tccatgacgttcctgacgtt

SEQ ID NO: 2 - DNA, 22 bp, Artificial Sequence
  Synthetic
  misc_feature (20)..(20): (SP18)2
tccatgacgttcctgacgtttt

SEQ ID NO: 3 - DNA, 20 bp, Artificial Sequence
  Synthetic
  misc_feature (20)..(20): THIOL
aacgtcaggaacgtcatgga

SEQ ID NO: 4 - DNA, 20 bp, Artificial Sequence
  Synthetic
  misc_feature (20)..(20): SP18 THIOL
aacgtcaggaacgtcatgga

SEQ ID NO: 5 - DNA, 20 bp, Artificial Sequence
  Synthetic
  misc_feature (1)..(1): THIOL SP18
aacgtcaggaacgtcatgga

SEQ ID NO: 6 - DNA, 24 bp, Artificial Sequence
  Synthetic
  misc_feature (24)..(24): (Spacer-18 (hexaethyleneglycol))2Cholesterol
tcgtcgttttgtcgttttgtcgtt

SEQ ID NO: 7 - protein, 8 aa, Artificial Sequence
  Synthetic
SIINFEKL

SEQ ID NO: 8 - protein, 17 aa, Artificial Sequence
  Synthetic
ISQAVHAAHAEINEAGR

SEQ ID NO: 9 - protein, 9 aa, Artificial Sequence
  Synthetic
LCPGNKYEM

SEQ ID NO: 10 - protein, 18 aa, Artificial Sequence
  Synthetic
CSSVDWENVSPELNSTDQ

SEQ ID NO: 11 - protein, 9 aa, Artificial Sequence
  Synthetic
KVPRNQDWL

SEQ ID NO: 12 - protein, 10 aa, Artificial Sequence
  Synthetic
VYDFAFRDLC

SEQ ID NO: 13 - protein, 9 aa, Artificial Sequence
  Synthetic
RAHYNIVTF

SEQ ID NO: 14 - protein, 9 aa, Artificial Sequence
  Synthetic
SAVKNFTEI

SEQ ID NO: 15 - protein, 9 aa, Artificial Sequence
  Synthetic
STHVNHLHC

SEQ ID NO: 16 - protein, 9 aa, Artificial Sequence
  Synthetic
SLDLKLIFL

SEQ ID NO: 17 - protein, 9 aa, Artificial Sequence
  Synthetic
SAMTNLAAL

SEQ ID NO: 18 - protein, 9 aa, Artificial Sequence
  Synthetic
ATGKNVCDV

SEQ ID NO: 19 - protein, 9 aa, Artificial Sequence
  Synthetic
ATGKNVCDM

SEQ ID NO: 20 - protein, 9 aa, Artificial Sequence
  Synthetic
TAPDNLGYA

SEQ ID NO: 21 - protein, 9 aa, Artificial Sequence
  Synthetic
TAPDNLGYM

SEQ ID NO: 22 - protein, 9 aa, Artificial Sequence
  Synthetic
SVYDFFVWL

SEQ ID NO: 23 - protein, 9 aa, Artificial Sequence
  Synthetic
ASMTNMELM

SEQ ID NO: 24 - protein, 9 aa, Artificial Sequence
  Synthetic
AALLNSAVL

SEQ ID NO: 25 - protein, 26 aa, Artificial Sequence
  Synthetic
KARDETAALLNSAVLGAAPLFVPPAD

SEQ ID NO: 26 - protein, 13 aa, Artificial Sequence
  Synthetic
RQLYPEWTEAQRL

SEQ ID NO: 27 - protein, 15 aa, Artificial Sequence
  Synthetic
QLLRREVYDFAFRDL

SEQ ID NO: 28 - protein, 15 aa, Artificial Sequence
  Synthetic
LKLIFLDSRVTEVTG

SEQ ID NO: 29 - protein, 15 aa, Artificial Sequence
  Synthetic
MSAMTNLAALFPPEG

SEQ ID NO: 30 - protein, 15 aa, Artificial Sequence
  Synthetic
VDWENVSPELNSTDQ

SEQ ID NO: 31 - protein, 15 aa, Artificial Sequence
  Synthetic
CRPGWRGAACNQKIL

SEQ ID NO: 32 - protein, 25 aa, Artificial Sequence
  Synthetic
SGHNCGTCRPGWRGAACNQKILTVR

SEQ ID NO: 33 - protein, 16 aa, Artificial Sequence
  Synthetic
LRMKLPKPPKPVSQMR

SEQ ID NO: 34 - protein, 15 aa, Artificial Sequence
  Synthetic
PAYEKLSAEQSPPPY

SEQ ID NO: 35 - protein, 14 aa, Artificial Sequence
  Synthetic
AAGIGILTVILGVL

SEQ ID NO: 36 - protein, 20 aa, Artificial Sequence
  Synthetic
RNGYRALMDKSLHVGTQCAL

SEQ ID NO: 37 - protein, 23 aa, Artificial Sequence
  Synthetic
RNGYRALMDKSLHVGTQCALTRR

SEQ ID NO: 38 - protein, 15 aa, Artificial Sequence
  Synthetic
IGCWYCRRRNGYRAL

SEQ ID NO: 39 - DNA, 20 bp, Artificial Sequence
  Synthetic
tccatgacgttcctgacgtt

SEQ ID NO: 40 - DNA, 24 bp, Artificial Sequence
  Synthetic
tcgtcgttttgtcgttttgtcgtt

SEQ ID NO: 41 - DNA, 20 bp, Artificial Sequence
  Synthetic
  misc_feature (20)..(20): (SPACER-18 (HEXAETHYLENEGLYCOL))2CHOLESTEROL
tccatgacgttcctgacgtt